(19) United States
(12) Patent Application Publication
(10) Pub. No.: US 2025/0345064 A1
(43) Pub. Date: Nov. 13, 2025

(54) Neck Bridge with Proximal Openings for Occluding an Aneurysm Sac

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 19/274,599

(22) Filed: Jul. 20, 2025

Related U.S. Application Data
(63) Continuation-in-part of application No. 19/023,514, filed on Jan. 16, 2025.
(63) Continuation-in-part of application No. 18/920,939, filed on Oct. 20, 2024.
Application No. 19/023,514 is a continuation-in-part of application No. 18/920,939, filed on Oct. 20, 2024.
Application No. 18/920,939 is a continuation-in-part of application No. 18/760,322, filed on Jul. 1, 2024, now abandoned.
Application No. 18/920,939 is a continuation-in-part of application No. 18/674,996, filed on May 27, 2024, now abandoned.
Application No. 18/760,322 is a continuation-in-part of application No. 18/674,996, filed on May 27, 2024, now abandoned.
Application No. 18/674,996 is a continuation-in-part of application No. 18/613,053, filed on Mar. 21, 2024, now abandoned.
Application No. 18/613,053 is a continuation-in-part of application No. 17/970,510, filed on Oct. 20, 2022, now abandoned.
Application No. 18/674,996 is a continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
(60) Provisional application No. 62/444,860, filed on Jan. 11, 2017.
(60) Provisional application No. 62/472,519, filed on Mar. 16, 2017.
(60) Provisional application No. 62/472,519, filed on Mar. 16, 2017.
(60) Provisional application No. 62/589,754, filed on Nov. 22, 2017.
(60) Provisional application No. 62/720,173, filed on Aug. 21, 2018.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 63/119,774, filed on Dec. 1, 2020.

(51) Int. Cl. A61B 17/12

(52) U.S. Cl. CPC A61B 17/12118; A61B 17/12168

(57) ABSTRACT

This invention is an intrasaccular aneurysm occlusion device with a neck bridge with one or more proximal openings which is inserted into an aneurysm sac to cover the neck of the aneurysm sac. Embolic pieces (or flowable material) are inserted through the openings into the aneurysm sac. The neck bridge can have a convex first shape which is collapsed and inverted into a concave second shape. The neck bridge can be used in combination with a flexible distal net or mesh.

### CROSS-REFERENCE TO RELATED APPLICATIONS

This application is a continuation-in-part of U.S. patent application Ser. No. 19/023,514 filed on 2025 Jan. 16 and a continuation-in-part of U.S. patent application Ser. No. 18/920,939 filed on 2024 Oct. 20. U.S. patent application Ser. No. 19/023,514 was a continuation-in-part of U.S. patent application Ser. No. 18/920,939 filed on 2024 Oct. 20 and a continuation-in-part of U.S. patent application Ser. No. 18/760,322 filed on 2024 Jul. 1.

U.S. patent application Ser. No. 18/920,939 was a continuation-in-part of U.S. patent application Ser. No. 18/760,322 filed on 2024 Jul. 1 and a continuation-in-part of U.S. patent application Ser. No. 18/674,996 filed on 2024 May 27. U.S. patent application Ser. No. 18/760,322 was a continuation-in-part of U.S. patent application Ser. No. 18/674,996 filed on 2024 May 27, a continuation-in-part of U.S. patent application Ser. No. 18/613,053 filed on 2024 Mar. 21, and a continuation-in-part of U.S. patent application Ser. No. 17/970,510 filed on 2022 Oct. 20.

U.S. patent application Ser. No. 18/674,996 was a continuation-in-part of U.S. Pat. No. 18,613,053 filed on 2024 Mar. 21 and a continuation-in-part of U.S. patent application Ser. No. 18/519,055 filed on 2023 Nov. 26. U.S. patent application Ser. No. 18/613,053 was a continuation-in-part of U.S. patent application Ser. No. 18/519,055 filed on 2023 Nov. 26 and a continuation-in-part of U.S. patent application Ser. No. 18/135,153 filed on 2023 Apr. 15.

U.S. patent application Ser. No. 18/519,055 was a continuation-in-part of U.S. patent application Ser. No. 18/374,602 filed on 2023 Sep. 28, a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/374,602 was a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/135,153 was a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31. U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11.

The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to aneurysm occlusion devices and methods.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Even among those who survive, approximately one-half suffer significant and permanent deterioration of brain function. Better alternatives for cerebral aneurysm treatment are needed.

### REVIEW OF THE RELEVANT ART

U.S. patent application publication 20150272589 (Lorenzo, Oct. 1, 2015, “Aneurysm Occlusion Device”) discloses a tubular structure with a control ring. U.S. Pat. No. 10,327,781 (Divino et al., Jun. 25, 2019, “Occlusive Devices”), U.S. Pat. No. 11,690,628 (Bardsley et al., Jul. 4, 2023, “Occlusive Devices”), U.S. Pat. No. 11,786,253 (Divino et al., Oct. 17, 2023, “Occlusive Devices”), and U.S. Pat. No. 12,193,675 (Divino et al., Jan. 14, 2025, “Occlusive Devices”) disclose expandable embolic structures with specific shapes and/or porosities. U.S. patent application publication 20190192168 (Lorenzo et al., Jun. 27, 2019, “Aneurysm Device and Delivery Method”) discloses a self-expanding braid which slides in a catheter in a collapsed state.

U.S. patent application publication 20190307546 (Aguilar et al., Oct. 10, 2019, “Embolic Device with Improved Neck Coverage”) discloses an embolic structure with a spiral shape. U.S. Pat. No. 10,653,425 (Gorochow et al., May 19, 2020, “Layered Braided Aneurysm Treatment Device”) discloses a tubular braid with a first segment from an open end to a first inversion, a second segment from the first inversion to a second inversion, and a third segment from the second inversion to a pinched end. U.S. patent application publication 20210128160 (Li et al., May 6, 2021, “Systems and Methods for Treating Aneurysms”) discloses a device comprising an expandable braid and embolic elements.

U.S. patent application publication 20210128167 (Patel et al., May 6, 2021, “Systems and Methods for Treating Aneurysms”) discloses an elongate tubular member with an engagement member which is removably coupled to a proximal hub. U.S. patent application publication 20210128169 (Li et al., May 6, 2021, “Devices, Systems, and Methods for Treatment of Intracranial Aneurysms”) discloses a method for deforming a shape of an occlusive member by inserting an embolic element between the occlusive member and an aneurysm wall. U.S. Pat. No. 11,071,551 (Garza et al., Jul. 27, 2021, “Flow Attenuation Device”) discloses an embolic device whose porosity varies along the length of the device.

U.S. patent application publication 20220031334 (Aguilar, Feb. 3, 2022, “Expandable Devices for Treating Body Lumens”) and U.S. Pat. No. 12,161,344 (Aguilar, Dec. 10, 2024, “Expandable Devices for Treating Body Lumens”) disclose an expandable mesh including an outer mesh and an inner mesh within the outer mesh. U.S. patent application publication 20230016312 (Xu et al., Jan. 19, 2023, “Aneurysm Treatment with Pushable Implanted Braid”) discloses a braided implant with a retractable dual proximal layer. U.S. patent application publication 20230277184 (Rashidi et al., Sep. 7, 2023, “Occlusive Devices with Thrombogenic Inserts”) discloses an insert between the upper and lower walls within an expandable mesh.

U.S. patent application publication 20240032941 (Shimizu et al., Feb. 1, 2024, “Embolic Material Delivery Device and Related Technology”) discloses an elongate conduit body defining an axial lumen through which a liquid embolic material is conveyed to an aneurysm. U.S. patent application publication 20240050099 (Pecor et al., Feb. 15, 2024, “Occlusive Devices for Treating Vascular Defects and Associated Systems and Methods”) discloses a mesh comprising at least two mesh layers and a membrane between the layers. U.S. patent application publication 20240065702 (Ogawa et al., Feb. 29, 2024, “Embolization Device”) discloses an outer tube having a distal end and a proximal end and a basket in a lumen of the outer tube.

U.S. patent application publication 20240075565 (Li et al., Mar. 7, 2024, “Systems and Methods for Treating Aneurysms”) discloses an electrolytically-corrodible core wire having a proximal portion, a distal portion, and a detachment zone between the proximal portion and the distal portion. U.S. patent application publication 20240099723 (Schabert et al., Mar. 28, 2024, “Intrasaccular Neck Bridging Device”) discloses a braided mesh body and at least one pinch member. U.S. patent application publication 20240206879 (Kandala et al., Jun. 27, 2024, “Occlusive Devices with Spiral Struts for Treating Vascular Defects”) discloses a plurality of spiral struts which are coupled to an anchor structure.

U.S. Pat. No. 12,029,431 (Griffin, Jul. 9, 2024, “Occlusion Device”) discloses an occlusion device for intrasaccular implantation with a substantially solid marker and a low profile resilient mesh body attached to a distal end of the marker. U.S. Pat. No. 12,053,182 (Aboytes et al., Aug. 6, 2024, “Devices and Methods for the Treatment of Vascular Defects”) discloses an expandable implant which can be moved from a first configuration in which a first portion and a second portion are substantially linearly aligned to a second configuration in which the second portion overlaps the first portion. U.S. Pat. No. 12,059,156 (Mayer et al., Aug. 13, 2024, “Devices for Treating Vascular Malformations”) discloses an apparatus with an orifice-section curve that winds at least 2.5 turns around an orifice-section central axis at a changing distance from the orifice-section central axis.

U.S. Pat. No. 12,070,220 (Dholakia et al., Aug. 27, 2024, “Devices Having Multiple Permeable Shells for Treatment of Vascular Defects”) discloses a plurality of permeable shells which are connected by a plurality of coils, wherein each coil connects at least one pair of permeable shells. U.S. Pat. No. 12,076,022 (Griffin, Sep. 3, 2024, “Occlusion Device”) discloses a continuous compressible mesh structure comprising axial mesh carriages configured end to end, wherein each end of each carriage is a pinch point in the continuous mesh structure. U.S. Pat. No. 12,082,821 (Marchand et al., Sep. 10, 2024, “Filamentary Devices for Treatment of Vascular Defects”) discloses a permeable shell having a radially-constrained elongated state within a catheter, an expanded state with a longitudinally-shortened configuration, and a plurality of elongate filaments that are woven together to form a mesh.

U.S. Pat. No. 12,096,940 (Hewitt et al., Sep. 24, 2024, “Filamentary Devices for Treatment of Vascular Defects”) discloses a self-expanding resilient permeable shell having a radially constrained state and an expanded state with a globular, axially shortened configuration. U.S. Pat. No. 12,102,327 (Pereira et al., Oct. 1, 2024, “Systems and Methods for Treating Aneurysms”) discloses an occlusion element comprising an inverted mesh tube with an outer layer and an inner layer, wherein the outer layer transitions to the inner layer at an inversion fold located at or adjacent the distal end of the occlusion element and the inversion fold defines an inner diameter. U.S. patent application publication 20240341769 (Bai et al., Oct. 17, 2024, “Intrasaccular Occlusive Devices Comprising Copper Materials”) discloses a mesh having a low-profile configuration for delivery through a catheter to an aneurysm sac and an expanded configuration for implantation in the aneurysm sac.

U.S. patent application publication 20240358376 (Khenansho, Oct. 31, 2024, “Systems and Methods for Occluding Vascular Defects”) discloses an occlusive device whose height is less than the height of an aneurysm such that a space exists between a distal surface of the occlusive member and a dome of the aneurysm. U.S. patent application publication 20240366226 (Gorochow et al., Nov. 7, 2024, “Braided Aneurysm Treatment Device with Flexible Inversion Region”) discloses a tubular braid with an open end, a pinched end, and a predetermined shape. U.S. patent application publication 20240366227 (Tran et al., Nov. 7, 2024, “Devices for Treatment of Vascular Defects”) discloses a permeable shell with an unrestrained preset configuration comprising a dome portion and a brim portion.

U.S. patent application publication 20240382208 (Tafti, Nov. 21, 2024, “Device for Vascular Occlusion and Methods of Use Thereof”) discloses arcuately-curved coils. U.S. Pat. No. 12,150,871 (Ruvalcaba et al., Nov. 26, 2024, “Occlusive Device”) discloses an aneurysm embolization device with an atraumatic tip portion extending from it. U.S. patent application publication 20240398412 (Zaidat et al., Dec. 5, 2024, “Systems and Methods for Treating Aneurysms”) discloses an inverted mesh tube having an outer layer and an inner layer, wherein the outer layer transitions to the inner layer at an inversion fold located at or adjacent the distal end of the occlusion element, and the inversion fold defines an inner diameter.

U.S. patent application publication 20250032121 (Rashidi et al., Jan. 30, 2025, “Systems and Methods for Treating Aneurysms”) discloses an occlusive implant, wherein expanding an expandable member is a balloon. U.S. patent application publication 20250041084 (Monstadt et al., Feb. 6, 2025, “Implant for Treating Aneurysms in the Area of Bifurcations”) discloses an implant with at least two branching tubular sections. U.S. patent application publication 20250049592 (Ruvalcaba et al., Feb. 13, 2025, “Occlusive Device”) discloses an expandable component and an atraumatic tip portion extending from it.

U.S. Pat. No. 12,256,936 (Li et al., Mar. 25, 2025, “Devices, Systems, and Methods for Treatment of Intracranial Aneurysms”) discloses a method for deforming the shape of an occlusive member by introducing an embolic element to a space between the occlusive member and an aneurysm wall. U.S. patent application publication 20250120654 (Salant et al., Apr. 17, 2025, “Implantable Medical Device with Sensing and Communication Functionality”) discloses an implantable system for monitoring a vascular structure with a sensor which transmits blood flow data. U.S. Pat. No. 12,303,136 (Mauger et al., May 20, 2025, “System and Methods for Embolized Occlusion of Neurovascular Aneurysms”) discloses an occlusion device which transitions between a two-dimensional configuration and a three-dimensional configuration.

U.S. patent application publication 20250169824 (Li et al., May 29, 2025, “Devices, Systems, and Methods for Treatment of Intracranial Aneurysms”) discloses a method for deforming the shape of the occlusive member by introduction of an embolic element to a space between the occlusive member and an aneurysm wall. U.S. patent application publication 20250176967 (Carrillo, Jun. 5, 2025, “Aneurysm Treatment Device and Associated Systems and Methods of Use”) discloses an aneurysm treatment device with a tip portion, a body portion, and a base portion.

### SUMMARY OF THE INVENTION

This invention is an intrasaccular aneurysm occlusion device with a neck bridge which is inserted into an aneurysm sac to cover the neck of the aneurysm sac. The neck bridge has one or more proximal openings through which embolic pieces (or flowable material) are inserted into the aneurysm sac. These openings can comprise a central opening and a plurality of non-central openings. In an example, the neck bridge can have a convex first shape which is collapsed and inverted into a concave second shape. In an example, the ends of the neck bridge can be bound together by concentric rings or bands. In an example, the neck bridge can be used in combination with a flexible distal net or mesh between the neck bridge and the dome of the aneurysm sac.

### BRIEF INTRODUCTION TO THE FIGURES

FIG. 1 shows a concave neck bridge with a central opening for inserting embolics.

FIG. 2 shows a concave neck bridge with non-central openings for inserting embolics.

FIG. 3 shows a two-layer concave neck bridge.

FIG. 4 shows a two-layer concave neck bridge with a central proximal-facing protrusion.

FIG. 5 shows a two-layer concave neck bridge with a central distal-facing protrusion.

FIG. 6 shows a partial-torus neck bridge with a central funnel that is shorter than its rim.

FIG. 7 shows a partial-torus neck bridge with a central funnel that is higher than its rim.

FIG. 8 shows compression and inversion of a convex stent into a concave stent.

FIG. 9 shows compression and inversion of a convex stent into a concave stent by pulling a wire.

FIG. 10 shows a convex stent made by connecting proximal and distal concave halves.

FIG. 11 shows a convex stent with proximal and distal halves made with different materials and/or structures.

FIG. 12 shows a concave stent with a rim band.

FIG. 13 shows a convex stent with a circumferential band.

FIG. 14 shows a convex stent with a circumferential band between proximal and distal portions.

FIG. 15 shows a convex stent with a circumferential band between proximal and distal portions which have different attributes.

FIG. 16 shows a torus-shaped stent.

FIG. 17 shows a torus-shaped stent with an opening to its interior.

FIG. 18 shows a torus-shaped stent made by inverting the ends of a tubular mesh.

FIG. 19 shows a stent with a torus shape modeled by revolving a tear-drop shape.

FIG. 20 shows a stent with a torus shape modeled by revolving a comma shape.

FIG. 21 shows a stent with a torus shape modeled by revolving a comma shape and an opening to the its interior.

FIG. 22 shows a stent with a torus shape modeled by revolving a comma shape wherein proximal ends curve distally.

FIG. 23 shows a stent whose distal surface has a proximally-pointing funnel and whose proximal surface has a distally-pointing funnel.

FIG. 24 shows a stent with a torus shape modeled by revolving a question mark.

FIG. 25 shows a stent with a cardioid shape nested within a bowl shape.

FIG. 26 shows a stent with a quasi “apple core” shape.

FIG. 27 shows a stent with a quasi “apple core” shape and a non-central proximal opening.

FIG. 28 shows a stent with a distal mesh or net partially nested within a proximal concave neck bridge.

FIGS. 29 and 30 show a distal convex stent and a proximal concave stent connected by a wire or cord.

FIG. 31 shows distal and proximal convex stents, wherein the proximal convex stent is compressed and inverted into a concave stent.

FIG. 32 shows a middle convex stent nested within proximal and distal concave stents.

FIG. 33 shows a stent with a cardioid shape partially nested in a double layer bowl shape.

FIG. 34 shows a convex stent within an outer mesh and proximal openings in the mesh.

FIG. 35 shows a convex stent within an outer mesh and proximal openings in the convex stent and the mesh.

FIG. 36 shows a convex stent spanning the circumference of an aneurysm sac within an outer mesh.

FIG. 37 shows a two-layer concave neck bridge and embolic pieces inserted through a central opening in the neck bridge.

FIG. 38 shows a collapsed and inverted two-layer concave neck bridge and embolic pieces inserted through a central opening in the neck bridge.

FIG. 39 shows a two-layer concave neck bridge and embolic coils inserted through a central opening in the neck bridge.

FIG. 40 shows a concave neck bridge and embolic flowable material (e.g. congealing liquid or gel) inserted through a central valve in the neck bridge.

FIG. 41 shows a two-layer concave neck bridge and embolic flowable material (e.g. congealing liquid or gel) inserted through a central opening in the neck bridge.

FIG. 42 shows a distal mesh or net nested within a concave neck bridge and a central opening in the neck bridge.

FIG. 43 shows a partial-torus neck bridge and a distal mesh or net.

FIG. 44 shows a concave neck bridge and a distal mesh or net into which embolic pieces have been inserted.

FIG. 45 shows a compressed and inverted concave neck bridge and a distal mesh or net into which embolic pieces have been inserted.

FIG. 46 shows a cross-sectional view of a concave neck bridge with a central opening and a valve, an annular constraint, and a distal mesh or net.

FIG. 47 shows a cross-sectional view of a concave neck bridge with a central opening and a valve, an annular constraint, a distal mesh or net, and embolic pieces inserted into the distal mesh or net.

FIG. 48 shows a cross-sectional view of a concave neck bridge with a central opening and a valve, an annular constraint, a distal mesh or net, and hydrogel pieces inserted into the distal mesh or net.

FIG. 49 shows a cross-sectional view of a concave neck bridge with a central opening and a valve, an annular constraint, a distal mesh or net, and embolic coils inserted into the distal mesh or net.

FIG. 50 shows a cross-sectional view of a concave neck bridge with a central opening and a valve, an annular constraint, a distal mesh or net, and embolic flowable material (e.g. congealing liquid or gel) inserted into the distal mesh or net.

FIG. 51 shows a cross-sectional view of a concave neck bridge with a central opening and a valve, an annular constraint, a distal mesh or net, and string-of-pearls embolic strands inserted into the distal mesh or net.

FIG. 52 shows a concave neck bridge with a guide wire through a central opening.

FIG. 53 shows a concave neck bridge and a column through the neck bridge.

FIG. 54 shows a concave neck bridge, a column through the neck bridge, and embolic pieces inserted through the column.

FIG. 55 shows a concave neck bridge, a column through the neck bridge, and embolic coils inserted through the column.

FIG. 56 shows a concave neck bridge, a column through the neck bridge, and embolic flowable material (e.g. congealing liquid or gel) inserted through the column.

FIG. 57 shows a concave neck bridge, a column through the neck bridge, and a distal mesh or net.

FIG. 58 shows a partial-torus neck bridge with an extended central funnel and a distal mesh or net.

FIG. 59 shows a convex neck bridge and an inner funnel which extends out distally from the neck bridge.

FIG. 60 shows a globular neck bridge and an inner column which extends out distally from the neck bridge.

FIG. 61 shows a convex neck bridge, an inner funnel which extends out distally from the neck bridge, and a distal mesh or net.

FIG. 62 shows a convex neck bridge, an inner funnel which extends out distally from the neck bridge, a distal mesh or net, and string-of-pearls embolic strands inserted through the funnel into the distal mesh or net.

FIG. 63 shows a device with an outer convex layer, an inner convex layer, and an inner column.

FIG. 64 shows a dumbbell-shaped neck bridge.

FIGS. 65 and 66 show a series of three convex stents which is longitudinally-compressed within an aneurysm sac.

FIGS. 67 and 68 show an undulating device with three bulges which is longitudinally-compressed within an aneurysm sac.

FIGS. 69 and 70 show a series of three convex stents which are selectively compressed and inverted within an aneurysm sac.

FIG. 71 shows a series of three directly-connected ellipsoidal stents.

FIG. 72 shows a series of three ellipsoidal stents which are connected by a wire or cord.

FIG. 73 shows a series of ellipsoidal and concave stents which are connected by a wire or cord.

FIG. 74 shows a series of concave stents which are connected by a wire or cord.

FIG. 75 shows an embolic helical coil.

FIG. 76 shows an embolic helical coil with an attached wire or cord.

FIG. 77 shows an embolic concave helical coil with an outer net or mesh layer.

FIG. 78 shows an embolic helical coil with an outer net or mesh layer.

FIG. 79 shows an embolic helical coil with an outer net or mesh layer filled with embolic pieces.

FIG. 80 shows a globular device with a proximal concave mesh and a distal concave helical coil.

FIG. 81 shows a globular device with a proximal concave helical coil and a distal concave helical coil.

FIG. 82 shows a globular device with a proximal concave helical coil and a distal concave helical coil, wherein the device is filled with embolic pieces.

FIG. 83 shows an embolic string-of-pearls strand with an alternating sequence of larger and smaller pieces.

FIG. 84 shows an embolic string-of-pearls strand wherein distal pieces are larger than proximal pieces.

FIG. 85 shows an embolic string-of-pearls strand wherein distal pieces are closer together than proximal pieces.

FIG. 86 shows an embolic string-of-pearls strand wherein distal pieces are less-compressible than proximal pieces.

FIG. 87 shows an embolic string-of-pearls strand wherein proximal pieces are more irregularly-shaped than distal pieces.

FIG. 88 shows an embolic string-of-pearls strand wherein pieces are connected by an undulating wire.

FIG. 89 shows an embolic string-of-pearls strand wherein pieces are connected by a coil.

FIG. 90 shows an embolic string-of-pearls strand wherein pieces can slide back and forth along a wire or cord.

FIGS. 91 and 92 show two views of a device comprising an inverting convex stent, a central column, and embolic pieces.

FIGS. 93 and 94 show two views of a device comprising an inverting convex stent, a central column, and embolic coils.

FIGS. 95 and 96 show two views of a device comprising an inverting convex stent, a central column, and embolic flowable material (e.g. congealing liquid or gel).

FIGS. 97 through 100 show four sequential views of deployment of an intrasaccular aneurysm occlusion device wherein a one-layer globular stent is collapsed and inverted into a two-layer concave stent.

FIG. 101 shows a partial-torus neck bridge whose ends are inserted between nested annular bands.

FIG. 102 shows a partial-torus neck bridge whose ends are inserted between nested annular bands and embolic pieces inserted into an aneurysm sac.

FIG. 103 shows a partial-torus neck bridge whose ends are inserted between nested annular bands and embolic coils inserted into an aneurysm sac.

FIGS. 104 and 105 show two views of a concave neck bridge with a central opening and a plurality of non-central openings.

FIG. 106 shows a concave neck bridge with a central opening and a plurality of non-central openings, wherein embolic pieces have been inserted through an opening.

FIG. 107 shows a concave neck bridge with a central opening and a plurality of non-central openings, wherein embolic coils have been inserted through an opening.

### DETAILED DESCRIPTION OF THE FIGURES

In an example, an intrasaccular aneurysm occlusion device can comprise: a proximal stent which is configured to be inserted into an aneurysm sac, wherein the proximal stent has a convex first configuration and a concave second configuration, and wherein the proximal stent is expanded into its convex first configuration within an aneurysm sac and then compressed and inverted into its concave second configuration across the aneurysm neck; a distal flexible mesh or net within the aneurysm sac; and embolic pieces which are (or flowable material which is) inserted into and retained within the distal flexible mesh or net.

In an example, a convex first configuration can be globular, spherical, and/or ball-shaped. In an example, a concave second configuration can be hemispherical and/or bowl-shaped. In an example, a stent can be compressed and inverted by pulling a wire. In an example, a stent can be compressed and inverted by pressure from insertion of embolic pieces or flowable material into the distal flexible mesh or net. In an example, a stent can have a proximal opening through which embolic pieces are or flowable material is inserted. In an example, this opening can be closed after embolic pieces are or flowable material have been inserted.

In an example, an intrasaccular aneurysm occlusion device can comprise: a partial-torus neck bridge which is configured to be inserted and expanded within an aneurysm sac to cover the neck of an aneurysm, wherein the neck bridge further comprises a central funnel, indentation, and/or column which extends distally from the proximal surface of the neck bridge; an inner annular member; and an outer annular member, wherein the inner annular member and the inner annular member are nested, wherein portions of the neck bridge are inserted between the inner annular member and the outer annular member, and wherein embolic pieces or flowable material can be inserted through a central opening of the inner annular member into the aneurysm sac.

In an example, the neck bridge can be overall concave, except for the central funnel, indentation, and/or column. In an example, the inner annular member can be a ring, band, washer, column, or tube. In an example, the outer annular member can be a ring, band, washer, column, or tube. In an example, the inner annular member and the outer annular member can be concentric and/or coaxial. In an example, the ends of the neck bridge can be held, bound, and/or pinched between the inner annular member and the outer annular member. In an example, the neck bridge can have two layers and the ends of the two layers can be held, bound, and/or pinched between the inner annular member and the outer annular member. In an example, the central funnel, indentation, or column of the neck bridge can be less distal than the circumferential rim of the neck bridge.

In an example, an intrasaccular aneurysm occlusion device can comprise: a convex neck bridge which is configured to be inserted and expanded within an aneurysm sac to cover the neck of the aneurysm; a central opening in the neck bridge through which embolic pieces or flowable material can be inserted into the aneurysm sac; and a plurality of non-central openings in the neck bridge through which embolic pieces or flowable material can be inserted into the aneurysm sac.

In an example, the neck bridge can have a plurality of pores in addition to the central opening and the non-central openings, wherein a pore is less than half the size of an opening. In an example, the neck bridge can have a plurality of pores in addition to the central opening and the non-central openings, wherein a pore is smaller than an embolic piece but an opening is larger than an embolic piece, so that embolic pieces can be inserted the aneurysm sac through an opening, but embolic pieces do not escape out of the aneurysm sac through a pore. In an example, a subset of the central opening and the non-central openings can be selectively and remotely opened by a device operator. In an example, the neck bridge can further comprise a plurality of valves and/or closure mechanisms which enable selective opening of a subset of the central opening and the non-central openings.

FIG. 1 shows an example of an intrasaccular aneurysm occlusion device comprising: a distally-concave (e.g. bowl-shaped, hemispherical, or half-ellipsoidal) neck bridge 102 which is configured to be inserted into and then radially-expanded within an aneurysm sac 101, wherein a post-expansion diameter of the distally-concave neck bridge is configured to be greater than a diameter of the aneurysm neck, and wherein there is a central opening (hole, lumen, or tube) 103 in a proximal surface of the distally-concave neck bridge through which embolic pieces (or flowable material) are inserted into the aneurysm sac.

In this disclosure, “proximal” means farther from the dome of the aneurysm sac (e.g. closer to the parent vessel of the aneurysm) and “distal” means closer to the dome of the aneurysm sac (e.g. farther from the parent vessel). In this disclosure, “distally-concave” means concave with its opening facing in a distal direction and its peak facing in a proximal direction.

In an example, a distally-concave neck bridge can have a bowl shape. In an example, a distally-concave neck bridge can have shape which is a section (e.g. between 30% and 70%) of the perimeter of a sphere. In an example, a distally-concave neck bridge can have a hemispherical shape. In an example, a distally-concave neck bridge can have shape which is a section (e.g. between 30% and 70%) of the perimeter of an ellipsoid. In an example, a distally-concave neck bridge can have a half-ellipsoidal shape. In an example, a distally-concave neck bridge can have shape which is a section (e.g. between 30% and 70%) of the perimeter of an torus. In an example, a distally-concave neck bridge can have a half-toroidal shape (e.g. the lower half like a cut bagel). In an example, a distally-concave neck bridge can have an inverted-umbrella shape.

In an example, a distally-concave neck bridge can be radially-compressed (and longitudinally-elongated) for delivery through a catheter to an aneurysm sac and then radially-expanded (and longitudinally-shortened) within the aneurysm sac. In an example, a distally-concave neck bridge can self-expand radially after exiting a catheter. In an example, a distally-concave neck bridge can be expanded-radially after exiting a catheter by a wire (or filament) connected to it which is pushed, pulled, or rotated. In an example, a distally-concave neck bridge can be expanded-radially after exiting a catheter by the application of electrical energy.

In an example, a distally-concave neck bridge can comprise a mesh, braid, or stent. In an example, a distally-concave neck bridge can comprise a woven or braided mesh, braid, or stent. In an example, a distally-concave neck bridge can comprise a 3D printed mesh, braid, or stent. In an example, a distally-concave neck bridge can comprise a laser-cut mesh, braid, or stent. In an example, a distally-concave neck bridge can be made from metal wires, tubes, and/or coils. In an example, a distally-concave neck bridge can be made from polymer strands, filaments, threads, or yarns. In an example, a distally-concave neck bridge can be made from organic strands, filaments, threads, or yarns. In an example, a distally-concave neck bridge can be made from metal(s), polymer(s), or a combination of metal(s) and polymer(s).

In an example, a distally-concave neck bridge can comprise a single layer. In an example, a distally-concave neck bridge can comprise two layers (e.g. a proximal layer and a distal layer). In an example, a distally-concave neck bridge can be formed by proximally compressing and inverting a convex (e.g. spherical) member into a concave (e.g. bowl-shaped) member. In an example, a distally-concave neck bridge can comprise two layers (e.g. proximal and distal layers) which are continuous with each other at a distal (e.g. radial perimeter) fold.

In an example, a distally-concave neck bridge can comprise two layers (e.g. proximal and distal layers) which are connected to each other by a proximal annular member (e.g. band, ring, hub, twisted wire, zip tie, cord, clip, or washer). In an example, a proximal annular member can bind together the proximal ends of an inverted and/or folded mesh or net that forms a neck bridge. In an example, a distally-concave neck bridge can comprise two layers (e.g. proximal and distal layers) which are connected to each other by two concentric proximal annular members (e.g. bands, rings, hubs, wires, cords, clips, or washers), wherein ends of the two layers are inserted between the two concentric proximal annual members.

In an example, a distally-concave neck bridge can comprise three layers, wherein the proximal and distal layers are portions of the same piece of material (e.g. the same mesh or braid) and a middle layer between the proximal and distal layers is a separate piece of material. In an example, the proximal and distal layers can be metal and the middle layer can be made from a polymer or organic-material. In an example, the middle layer can comprise a membrane, film, mesh, or filter.

In an example, an opening (e.g. hole, lumen, or tube) in a proximal surface of a distally-concave neck bridge for delivering embolic pieces (or flowable material) can be located at a proximal center of the neck bridge. In an example, an opening in the proximal surface of a distally-concave neck bridge can be through the interior (e.g. central lumen) of a proximal annular member (e.g. band or ring) which connects proximal portions of the proximal and distal layers of the neck bridge. In an example, an opening in the proximal surface of a distally-concave neck bridge can be through the interior (e.g. central opening) of concentric proximal annular members (e.g. bands or rings) which connect portions of proximal and distal layers of the neck bridge.

In an example, a distally-concave neck bridge can comprise a mesh or net with a plurality of pores, holes, or gaps. In an example, these pores, holes, or gaps can be hexagonal. In an example, these pores, holes, or gaps can be triangular. In an example, these pores, holes, or gaps can be circular. In an example, embolic pieces which are inserted through a central opening in the distally-concave neck bridge can be larger than (e.g. at least twice as large as) any of the pores, holes, or gaps in the mesh or net. In an example, a proximal opening in the distally-concave neck bridge can be larger than (e.g. at least twice as large as) any of the pores, holes, or gaps in the mesh or net. In an example, a device can comprise non-central openings in the proximal surface of a distally-concave neck bridge through which embolic pieces (or flowable material) can be inserted into an aneurysm sac, wherein these two or more non-central openings are larger than (e.g. at least twice as large as) any of the (other) pores, holes, or gaps in the mesh or net.

In an example, a device can further comprise a closure mechanism (e.g. valve, plug, clip, or loop) which can be used (e.g. controlled by a device operator) to close the opening after embolic pieces (or flowable material) have been inserted through it into the aneurysm sac. In an example, a closure mechanism can be activated by the application of electrical energy, by pulling or pushing a wire, by rotating a wire, by pushing a plug, or by pumping congealing material. In an example, embolic pieces (or flowable material) which are inserted through an opening into an aneurysm sac can be selected from the group consisting of: micro-sponges; compressible micro-balls; embolic beads; pieces of foam; pieces of hydrogel; string-of-pearls embolic strands (e.g. embolic pieces connected by flexible longitudinal filaments, wires, coils, or threads); embolic coils; embolic ribbons; and liquid embolic material (e.g. flowable liquid or gel that congeals within the sac). Relevant variations discussed elsewhere in this disclosure or in priority-linked disclosures can also be applied to this example.

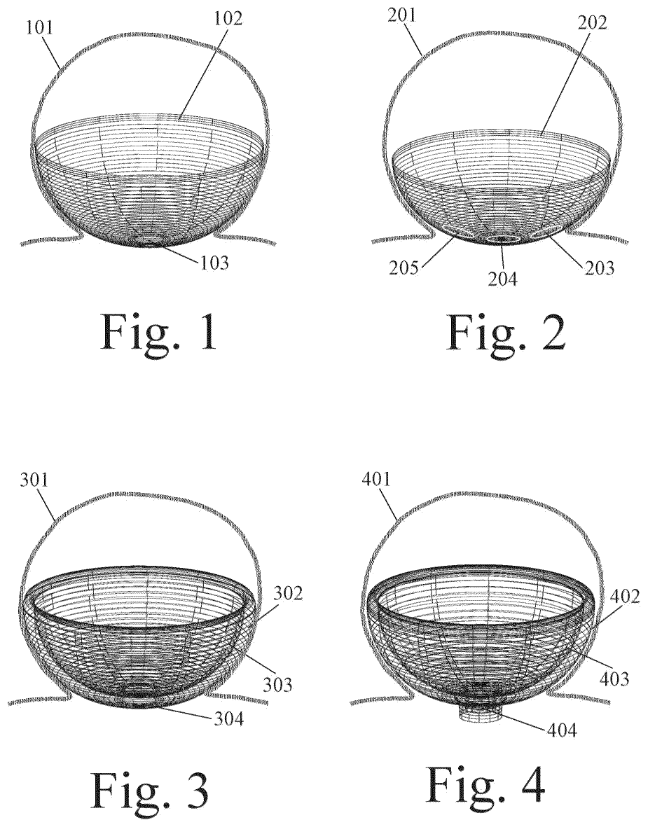

FIG. 2 shows an example of an intrasaccular aneurysm occlusion device which is like the one shown in FIG. 1 except that it also includes non-central openings in the proximal surface of the distally-concave neck bridge through which embolic pieces (or flowable material) are inserted into the aneurysm sac.

FIG. 2 shows an example of an intrasaccular aneurysm occlusion device comprising: a distally-concave (e.g. bowl-shaped, hemispherical, or half-ellipsoidal) neck bridge 202 which is configured to be inserted into and then radially-expanded within an aneurysm sac 201, wherein a post-expansion diameter of the distally-concave neck bridge is configured to be greater than a diameter of the aneurysm neck, wherein there is a central opening 204 in a proximal surface of the distally-concave neck bridge through which embolic pieces (or flowable material) are inserted into the aneurysm sac; and wherein there are also one or more non-central openings 203 and 205 in the proximal surface of the distally-concave neck bridge through which embolic pieces (or flowable material) are inserted into the aneurysm sac. In an example, one or more of the non-central openings can be selectively opened or closed by the operator to selectively deliver embolic pieces (or flowable material) into the aneurysm sac through different openings. Relevant variations discussed elsewhere in this disclosure or in priority-linked disclosures can also be applied to this example.

FIG. 3 shows an example of an intrasaccular aneurysm occlusion device comprising: a two-layer distally-concave (e.g. bowl-shaped, hemispherical, or half-ellipsoidal) neck bridge further comprising proximal layer 302 and distal layer 303, wherein the distally-concave neck bridge is configured to be inserted into and then radially-expanded within an aneurysm sac 301, wherein a post-expansion diameter of the distally-concave neck bridge is configured to be greater than a diameter of the aneurysm neck, and wherein there are central openings 304 in the proximal and distal layers through which embolic pieces (or flowable material) are inserted into the aneurysm sac.

In an example, a two-layer distally-concave neck bridge can have a bowl shape. In an example, a two-layer distally-concave neck bridge can have shape which is a section (e.g. between 30% and 70%) of the perimeter of a sphere. In an example, a two-layer distally-concave neck bridge can have a hemispherical shape. In an example, a two-layer distally-concave neck bridge can have shape which is a section (e.g. between 30% and 70%) of the perimeter of an ellipsoid. In an example, a two-layer distally-concave neck bridge can have a half-ellipsoidal shape. In an example, a two-layer distally-concave neck bridge can have shape which is a section (e.g. between 30% and 70%) of the perimeter of an torus. In an example, a two-layer distally-concave neck bridge can have a half-toroidal shape (e.g. the lower half like a cut bagel). In an example, a two-layer distally-concave neck bridge can have an inverted-umbrella shape.

In an example, a two-layer distally-concave neck bridge can be radially-compressed (and longitudinally-elongated) for delivery through a catheter to an aneurysm sac and then radially-expanded (and longitudinally-shortened) within the aneurysm sac. In an example, a two-layer distally-concave neck bridge can self-expand radially after exiting a catheter. In an example, a two-layer distally-concave neck bridge can be expanded-radially after exiting a catheter by a wire (or filament) connected to it which is pushed, pulled, or rotated. In an example, a two-layer distally-concave neck bridge can be expanded-radially after exiting a catheter by the application of electrical energy.

In an example, a two-layer distally-concave neck bridge can comprise a mesh, braid, or stent. In an example, a two-layer distally-concave neck bridge can comprise a woven or braided mesh, braid, or stent. In an example, a two-layer distally-concave neck bridge can comprise a 3D printed mesh, braid, or stent. In an example, a two-layer distally-concave neck bridge can comprise a laser-cut mesh, braid, or stent. In an example, a two-layer distally-concave neck bridge can be made from metal wires, tubes, and/or coils. In an example, a two-layer distally-concave neck bridge can be made from polymer strands, filaments, threads, or yarns. In an example, a two-layer distally-concave neck bridge can be made from organic strands, filaments, threads, or yarns. In an example, a two-layer distally-concave neck bridge can be made from metal(s), polymer(s), or a combination of metal(s) and polymer(s).

In an example, a two-layer distally-concave neck bridge can be formed by proximally compressing and inverting a convex (e.g. spherical) member into a concave (e.g. bowl-shaped) member. In an example, a two-layer distally-concave neck bridge can comprise two layers (e.g. proximal and distal layers) which are continuous with each other at a distal (e.g. radial perimeter) fold. In an example, a two-layer distally-concave neck bridge can comprise two layers (e.g. proximal and distal layers) which are connected to each other by a proximal annular member (e.g. band, ring, hub, twisted wire, zip tie, cord, clip, or washer). In an example, a proximal annular member can bind together the proximal ends of an inverted and/or folded mesh or net that forms a neck bridge. In an example, a two-layer distally-concave neck bridge can comprise two layers (e.g. proximal and distal layers) which are connected to each other by two concentric proximal annular members (e.g. bands, rings, hubs, wires, cords, clips, or washers), wherein ends of the two layers are inserted between the two concentric proximal annual members.

In an example, there can be a uniform distance between proximal and distal layers of a distally-concave neck bridge. In an example, the distance between proximal and distal layers of a distally-concave neck bridge can increase with distance from the aneurysm neck. In an example, the distance between proximal and distal layers of a distally-concave neck bridge can increase and then decrease with distance from the aneurysm neck. In an example, the distance between proximal and distal layers of a distally-concave neck bridge can decrease with distance from the aneurysm neck.

In an example, a distally-concave neck bridge can comprise three layers, wherein the proximal and distal layers are portions of the same piece of material (e.g. the same mesh or braid) and a middle layer between the proximal and distal layers is a separate piece of material. In an example, the proximal and distal layers can be metal and the middle layer can be made from a polymer or organic-material. In an example, the middle layer can comprise a membrane, film, mesh, or filter.

In an example, openings (e.g. holes, lumens, or tubes) in proximal and distal layers of a two-layer distally-concave neck bridge can be located at the proximal centers of these layers. In an example, openings (e.g. holes, lumens, or tubes) in proximal and distal layers of a two-layer distally-concave neck bridge can be aligned with each other. In an example, an openings in proximal and distal layers can be through the interior (e.g. central lumen) of a proximal annular member (e.g. band or ring) which connects proximal portions of the proximal and distal layers of the concave member. In an example, openings in proximal and distal layers can be through the interior (e.g. central opening) of concentric proximal annular members (e.g. bands or rings) which connect portions of proximal and distal layers of the concave member.

In an example, a two-layer distally-concave neck bridge can comprise a mesh or net with a plurality of pores, holes, or gaps. In an example, these pores, holes, or gaps can be hexagonal. In an example, these pores, holes, or gaps can be triangular. In an example, these pores, holes, or gaps can be circular. In an example, embolic pieces which are inserted through a central opening in the two-layer distally-concave neck bridge can be larger than (e.g. at least twice as large as) any of the pores, holes, or gaps in the mesh or net. In an example, a proximal opening in the two-layer distally-concave neck bridge can be larger than (e.g. at least twice as large as) any of the pores, holes, or gaps in the mesh or net. In an example, a device can comprise non-central openings in the proximal surface of a two-layer distally-concave neck bridge through which embolic pieces (or flowable material) can be inserted into an aneurysm sac, wherein these two or more non-central openings are larger than (e.g. at least twice as large as) any of the (other) pores, holes, or gaps in the mesh or net.

In an example, a device can further comprise a closure mechanism (e.g. valve, plug, clip, or loop) which can be used (e.g. controlled by a device operator) to close the opening after embolic pieces (or flowable material) have been inserted through it into the aneurysm sac. In an example, a closure mechanism can be activated by the application of electrical energy, by pulling or pushing a wire, by rotating a wire, by pushing a plug, or by pumping congealing material. In an example, embolic pieces (or flowable material) which are inserted through an opening into an aneurysm sac can be selected from the group consisting of: micro-sponges; compressible micro-balls; embolic beads; pieces of foam; pieces of hydrogel; string-of-pearls embolic strands (e.g. embolic pieces connected by flexible longitudinal filaments, wires, coils, or threads); embolic coils; embolic ribbons; and liquid embolic material (e.g. flowable liquid or gel that congeals within the sac). Relevant variations discussed elsewhere in this disclosure or in priority-linked disclosures can also be applied to this example.

FIG. 4 shows an example of an intrasaccular aneurysm occlusion device which is like the one shown in FIG. 3 except that portions of the two layers protrude concentrically and proximally (e.g. in a concentric configuration and in a proximal direction) from a central proximal portion of the overall bowl shape of the neck bridge. In an example, these protruding portions can (partially) stick out from the neck bridge into the parent vessel of the aneurysm. In an example, these protruding portions can stick out from an annular member (e.g. ring, band, or twisted wire) which binds the proximal ends of a mesh or net that forms the two-layer neck bridge.

FIG. 4 shows an example of an intrasaccular aneurysm occlusion device comprising: a two-layer distally-concave (e.g. bowl-shaped, hemispherical, or half-ellipsoidal) neck bridge further comprising proximal layer 402 and distal layer 403, wherein the distally-concave neck bridge is configured to be inserted into and radially-expanded within an aneurysm sac 401, wherein a post-expansion diameter of the distally-concave neck bridge is configured to be greater than a diameter of the aneurysm neck, wherein protruding portions 404 of the proximal and distal layers protrude in a concentric configuration and in a proximal direction from a central portion of the concave member, and wherein there are openings in the protruding portions through which embolic pieces (or flowable material) are inserted into the aneurysm sac.

In an example, proximal portions of proximal and distal layers can be connected to each other by a proximal annular member (e.g. band, ring, hub, twisted wire, zip tie, or cord). In an example, protruding portions of the proximal and distal layers can protrude proximally from this annular member. In an example, these protruding portions can (partially) stick out from the neck bridge into the parent vessel of the aneurysm. In an example, these protruding portions can stick out from an annular member which binds the proximal ends of a mesh or net that forms the neck bridge.

In an example, the distally-concave neck bridge can have an overall bowl shape, except for the protruding portions which extend out proximally from center of the bowl shape. In an example, proximal portions of proximal and distal layers can be connected to each other by insertion between two nested (e.g. concentric) annular members (e.g. bands or rings) at a proximal location. In an example, protruding portions can protrude proximally from these concentric annular members.

FIG. 4 can also be described as showing an example of an intrasaccular aneurysm occlusion device comprising: a first part of a two-layer device, wherein the first part has concave (e.g. bowl, hemispherical, or half-ellipsoidal) shape, wherein the first part further comprises a proximal layer and a distal layer, wherein the first part is configured to be inserted into and then radially-expanded within an aneurysm sac, wherein a post-expansion diameter of the first part is configured to be greater than a diameter of the aneurysm neck; and a second part of the two-layer device, wherein the second part comprises concentric and proximal protrusions of the proximal and distal layers from a proximal central portion of the first part, and wherein embolic pieces (or flowable material) are inserted into the aneurysm sac through one or more openings in the second part. Relevant variations discussed elsewhere in this disclosure or in priority-linked disclosures can also be applied to this example.

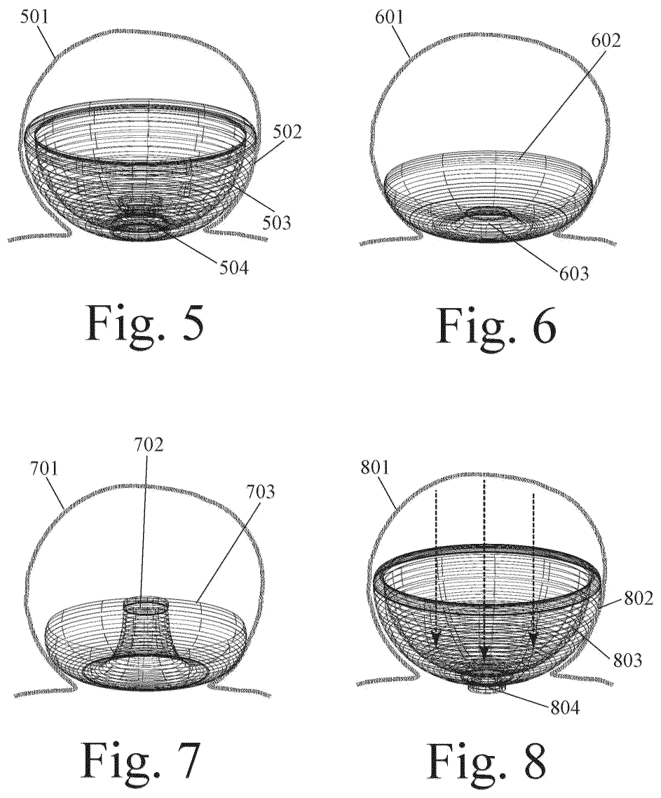

FIG. 5 shows an example of an intrasaccular aneurysm occlusion device which is like the one shown in FIG. 3 except that portions of the two layers protrude concentrically and distally (e.g. in a concentric configuration and in a distal direction) from a central proximal portion of the overall bowl shape of the neck bridge. In an example, these protruding portions can stick up from the bottom of the bowl shape into the aneurysm sac. In an example, these protruding portions can stick up from an annular member (e.g. ring, band, or twisted wire) which binds the proximal ends of a mesh or net that forms the two-layer neck bridge.

FIG. 5 shows an example of an intrasaccular aneurysm occlusion device comprising: a two-layer distally-concave (e.g. bowl-shaped, hemispherical, or half-ellipsoidal) neck bridge further comprising proximal layer 502 and distal layer 503, wherein the distally-concave neck bridge is configured to be inserted into and then radially-expanded within an aneurysm sac 501, wherein a post-expansion diameter of the distally-concave neck bridge is configured to be greater than a diameter of the aneurysm neck, wherein protruding portions 504 of the proximal and distal layers protrude in a concentric configuration and in a distal direction from a central portion of the concave member, and wherein there are openings in the protruding portions through which embolic pieces (or flowable material) are inserted into the aneurysm sac.

In an example, proximal portions of proximal and distal layers can be connected to each other by a proximal annular member (e.g. band, ring, hub, twisted wire, zip tie, or cord). In an example, protruding portions of the proximal and distal layers can protrude distally from this annular member. In an example, these protruding portions can (partially) stick up from the bottom of a bowl-shaped neck bridge into the aneurysm sac. In an example, these protruding portions can stick up from an annular member which binds the proximal ends of a mesh or net that forms the neck bridge.

In an example, the distally-concave neck bridge can have an overall bowl shape, except for the protruding portions which extend up distally from center of the bowl shape. In an example, proximal portions of proximal and distal layers can be connected to each other by insertion between two nested (e.g. concentric) annular members (e.g. bands or rings) at a proximal location. In an example, protruding portions can protrude distally from these concentric annular members.

FIG. 5 can also be described as showing an example of an intrasaccular aneurysm occlusion device comprising: a first part of a two-layer device, wherein the first part has concave (e.g. bowl, hemispherical, or half-ellipsoidal) shape, wherein the first part further comprises a proximal layer and a distal layer, wherein the first part is configured to be inserted into and then radially-expanded within an aneurysm sac, wherein a post-expansion diameter of the first part is configured to be greater than a diameter of the aneurysm neck; and a second part of the two-layer device, wherein the second part comprises concentric and distal protrusions of the proximal and distal layers from a proximal central portion of the first part, and wherein embolic pieces (or flowable material) are inserted into the aneurysm sac through one or more openings in the second part. Relevant variations discussed elsewhere in this disclosure or in priority-linked disclosures can also be applied to this example.

FIG. 6 shows an example of an intrasaccular aneurysm occlusion device comprising: a partial-torus neck bridge 602 which is configured to be inserted into and then radially-expanded within an aneurysm sac 601, wherein a post-expansion diameter of the partial-torus neck bridge is configured to be greater than a diameter of the aneurysm neck, and wherein there is an opening 603 in a central distal-pointing funnel, frustum, and/or indentation of the partial-torus neck bridge through which embolic pieces (or flowable material) are inserted into the aneurysm sac.

In an example, a full torus can be modeled as the revolution of a convex shape (e.g. circle or ellipse) around an axis which is coplanar with the shape, but outside the shape. In an example, a full torus can be modeled as the revolution of a circle around an axis which is coplanar with the circle, but outside the circle. A partial-torus is a section of a torus. In an example, a partial torus can be modeled as the revolution of a section of a convex shape around an axis which is coplanar with the section, but outside (the concavity of) the section. In an example, a half torus can be modeled as the revolution of a hemisphere around an axis which is coplanar with the hemisphere, but outside the concavity of the hemisphere. In an example, a quarter torus can be modeled as the revolution of a quarter of a circle around an axis which is coplanar with the hemisphere, but outside the concavity of the quarter.

In an example, a neck bridge can have a partial-torus shape. In an example, the shape of a partial-torus neck bridge can be modeled as the revolution of a section of a convex shape (e.g. circle or ellipse) around an axis which is coplanar to the section, but outside (the concavity of) the section. In an example, the shape of a partial-torus neck bridge can be modeled as the revolution of a section of a convex shape (e.g. circle or ellipse) around an axis which is coplanar to the section, but outside (the concavity of) the section, wherein the section is an oblique section (e.g. oblique hemisphere) of the convex shape.

In an example, the shape of a partial-torus neck bridge can be modeled as the revolution of a section of a convex shape (e.g. circle or ellipse) around an axis which is coplanar to the section, but outside (the concavity of) the section, wherein the section comprises between 25% and 50% of the perimeter of the convex shape. In an example, the shape of a partial-torus neck bridge can be modeled as the revolution of a section of a convex shape (e.g. circle or ellipse) around an axis which is coplanar to the section, but outside (the concavity of) the section, wherein the section comprises between 30% and 65% of the perimeter of the convex shape. In an example, the shape of a partial-torus neck bridge can be modeled as the revolution of a section of a convex shape (e.g. circle or ellipse) around an axis which is coplanar to the section, but outside (the concavity of) the section, wherein the section comprises between 50% and 80% of the perimeter of the convex shape.

In an example, a partial-torus neck bridge can be described as having a general bowl (e.g. hemispherical) shape, but further comprising a central funnel, frustum, or indentation which protrudes distally (e.g. upward) from the bottom of the bowl. In an example, the central funnel, frustum, or indentation can have a hyperbolic or half-hyperbolic shape. In an example, a partial-torus neck bridge can be described as having a general bowl (e.g. hemispherical) shape, but further comprising a central funnel, frustum, or indentation which protrudes distally (e.g. upward) from the bottom of the bowl, wherein the rim of the bowl is more distal than the peak of the funnel, frustum, or indentation extending up from the bottom of the bowl. In an example, a partial-torus neck bridge can be described as having a general bowl (e.g. hemispherical) shape, but further comprising a central funnel, frustum, or indentation which protrudes distally (e.g. upward) from the bottom of the bowl, wherein the rim of the bowl is at least twice as distal as the peak of the funnel, frustum, or indentation extending up from the bottom of the bowl.

In an example, a partial-torus neck bridge can have a shape which is like the lower-half of a torus (e.g. a cut bagel), except that a distal-pointing funnel or frustum of the half-torus is distally truncated (e.g. cut off) so that the funnel or frustum is shorter than (e.g. not as distal as) the circumferential rim of the half-torus. In an example, a partial-torus neck bridge can have a shape which is like the lower-half of a torus (e.g. a cut bagel), except that a distal-pointing funnel or frustum of the half-torus is distally truncated (e.g. cut off) so that the funnel or frustum is less than half the height of the circumferential rim of the half-torus.

In an example, the shape of a partial-torus neck bridge can be modeled as a generally bowl, hemispherical shape, or half-ellipsoidal shape with a central distal-pointing funnel, frustum, or indentation, wherein the diameter of the central distal-pointing funnel, frustum, or indentation is between 10% and 40% of the diameter of the rim of the partial-torus neck bridge. In an example, the shape of a partial-torus neck bridge can be modeled as a generally bowl, hemispherical shape, or half-ellipsoidal shape with a central distal-pointing funnel, frustum, or indentation, wherein the diameter of the central distal-pointing funnel, frustum, or indentation is between 35% and 65% of the diameter of the rim of the partial-torus neck bridge.

In an example, the shape of a partial-torus neck bridge can be modeled as a generally bowl, hemispherical shape, or half-ellipsoidal shape with a central local convexity, wherein the diameter of the central local convexity is between 10% and 40% of the diameter of the rim of the partial-torus neck bridge. In an example, the shape of a partial-torus neck bridge can be modeled as a generally bowl, hemispherical shape, or half-ellipsoidal shape with a central local convexity, wherein the diameter of the central local convexity is between 35% and 65% of the diameter of the rim of the partial-torus neck bridge.

In an example, an opening (e.g. hole, lumen, or tube) in a partial-torus neck bridge through which embolic pieces (or flowable material) are inserted can be through a central distal-pointing funnel, frustum, or indentation. In an example, the device can further comprise a closure mechanism (e.g. valve or plug) on the distal end of an opening through the central distal-pointing funnel, frustum, or indentation. Relevant variations discussed elsewhere in this disclosure or in priority-linked disclosures can also be applied to this example.

FIG. 7 shows an example of an intrasaccular aneurysm occlusion device comprising a partial-torus neck bridge. This example is similar to the one shown in FIG. 6 except that the central funnel, frustum, or indentation is more distal (e.g. higher) than the circumferential rim of the neck bridge.

FIG. 7 shows an example of an intrasaccular aneurysm occlusion device comprising: a partial-torus neck bridge 703 which is configured to be inserted into and then radially-expanded within an aneurysm sac 701, wherein a post-expansion diameter of the partial-torus neck bridge is configured to be greater than a diameter of the aneurysm neck, and wherein there is an opening 702 in a central distal-pointing funnel, frustum, and/or indentation of the partial-torus neck bridge through which embolic pieces (or flowable material) are inserted into the aneurysm sac.

In an example, a partial torus can be modeled as the revolution of a section of a convex shape around an axis which is coplanar with the section, but outside (the concavity of) the section. In this example, the shape of a partial-torus neck bridge can be modeled as the revolution of a section of a convex shape (e.g. circle or ellipse) around an axis which is coplanar to the section, but outside the concavity of the section, wherein the section comprises between 40% and 70% of the perimeter of the convex shape. In this example, the section is an oblique section, causing the peak of the central funnel, frustum, or indentation to be more distal (e.g. higher) than the circumferential rim of the neck bridge.

In an example, an opening in a partial-torus neck bridge through which embolic pieces (or flowable material) are inserted into the aneurysm sac is through the central distal-pointing funnel, frustum, or indentation. In an example, the device can further comprise a closure mechanism (e.g. valve or plug) on the distal end of on opening through the central distal-pointing funnel, frustum, or indentation. Relevant variations discussed elsewhere in this disclosure or in priority-linked disclosures can also be applied to this example.

FIG. 8 shows a key step in the formation of a two-layer concave neck bridge from a one-layer ball stent (e.g. convex mesh, braid, matrix, or frame). FIG. 8 shows this key step which comprises: proximally compressing and inverting a one-layer ball stent (e.g. convex mesh, braid, matrix, or frame) within an aneurysm sac 801 into a two-layer proximally-concave (e.g. bowl-shaped) neck bridge. The resulting neck bridge has a proximal layer 802 and a distal layer 803 which are continuous with each other along a distal circumferential fold (e.g. bowl rim). This neck bridge also includes openings 804 in the proximal and distal layers through which embolic pieces (or flowable material) can be inserted into the aneurysm sac. In this example, a distal half of a ball stent is proximally compressed and inverted into the concavity of a proximal half of the ball stent to create a two-layer bowl-shaped neck-bridge.

In an example, a one-step method for forming an intrasaccular occlusion device can comprise proximally compressing and inverting a distal portion (e.g. the distal half) of a ball stent (e.g. a convex mesh, braid, matrix, or frame) into a concavity of a proximal portion (e.g. the proximal half) of the ball stent in order to form a two-layer distally-concave neck bridge. In an example, a one-step method for forming an intrasaccular occlusion device can comprise proximally compressing and inverting a distal portion (e.g. the distal half) of a mesh ball, braid ball, or ball stent into a concavity of a proximal portion (e.g. the proximal half) of a mesh ball, braid ball, or ball stent to form a two-layer bowl-shaped mesh, braid, or stent.

In an example, a two-step method for forming an intrasaccular occlusion device can comprise: (a) radially-expanding a ball stent (e.g. convex mesh, braid, matrix, or frame) within an aneurysm sac; and (b) proximally compressing and inverting a distal portion (e.g. the distal half) of the ball stent (e.g. convex mesh, braid, matrix, or frame) into a concavity of a proximal portion (e.g. the proximal half) of the ball stent (e.g. convex mesh, braid, matrix, or frame) to form a two-layer distally-concave neck bridge. In an example, a two-step method for forming an intrasaccular occlusion device can comprise: (a) radially-expanding a mesh ball, braid ball, or ball stent within an aneurysm sac; and (b) proximally compressing and inverting a distal portion (e.g. the distal half) of the mesh ball, braid ball, or ball stent into a concavity of a proximal portion (e.g. the proximal half) of the mesh ball, braid ball, or ball stent to form a two-layer bowl-shaped mesh, braid, or stent.

In an example, a three-step method for forming an intrasaccular occlusion device can comprise: (a) radially-compressing a ball stent (e.g. convex mesh, braid, matrix, or frame) and inserting it into a catheter; (b) radially-expanding the ball stent (e.g. convex mesh, braid, matrix, or frame) after it has exited the catheter in an aneurysm sac; and (c) proximally compressing and inverting a distal portion (e.g. the distal half) of the ball stent (e.g. convex mesh, braid, matrix, or frame) into a concavity of a proximal portion (e.g. the proximal half) of the ball stent (e.g. convex mesh, braid, matrix, or frame) to form a two-layer distally-concave neck bridge.

In an example, a three-step method for forming an intrasaccular occlusion device can comprise: (a) radially-compressing a mesh ball, braid ball, or ball stent and inserting it into a catheter; (b) radially-expanding the mesh ball, braid ball, or ball stent after it has exited the catheter in an aneurysm sac; and (c) proximally compressing and inverting a distal portion (e.g. the distal half) of the mesh ball, braid ball, or ball stent into a concavity of a proximal portion (e.g. the proximal half) of the mesh ball, braid ball, or ball stent to form a two-layer distally-bowl-shaped mesh, braid, or stent.

In an example, a four-step method for forming an intrasaccular occlusion device can comprise: (a) radially-compressing a ball stent (e.g. convex mesh, braid, matrix, or frame) and inserting it into a catheter; (b) delivering the ball stent (e.g. convex mesh, braid, matrix, or frame) through the catheter to an aneurysm sac; (c) radially-expanding the ball stent (e.g. convex mesh, braid, matrix, or frame) in the aneurysm sac after it has exited the catheter; and (d) forming a two-layer distally-concave neck bridge within the aneurysm sac by proximally compressing and inverting a distal portion (e.g. the distal half) of the ball stent (e.g. convex mesh, braid, matrix, or frame) into a concavity of a proximal portion (e.g. the proximal half) of the ball stent (e.g. convex mesh, braid, matrix, or frame).

In an example, a four-step method for forming an intrasaccular occlusion device can comprise: (a) radially-compressing a mesh ball, braid ball, or ball stent and inserting it into a catheter; (b) delivering the mesh ball, braid ball, or ball stent through the catheter to an aneurysm sac; (c) radially-expanding the mesh ball, braid ball, or ball stent in the aneurysm sac after it has exited the catheter; and (d) forming a two-layer bowl-shaped mesh, braid, or stent within the aneurysm sac by proximally compressing and inverting a distal portion (e.g. the distal half) of the mesh ball, braid ball, or ball stent into a concavity of a proximal portion (e.g. the proximal half) of the mesh ball, braid ball, or ball stent.

In an example, a device can comprise a pre-compression (e.g. pre-inversion) ball stent with a spherical, ellipsoidal, apple-shaped, barrel-shaped, or egg shape. In an example, the device can comprise a post-compression (post-inversion) neck bridge with a bowl, hemispherical, half-ellipsoidal, or partial-torus shape. In an example, there can be a uniform distance between proximal and distal layers of the post-compression neck bridge. In an example, the distance between proximal and distal layers of the post-compression neck bridge can increase with distance from the aneurysm neck. In an example, the distance between proximal and distal layers of the post-compression neck bridge can increase and then decrease with distance from the aneurysm neck. In an example, the distance between proximal and distal layers of the post-compression neck bridge can decrease with distance from the aneurysm neck.

In an example, a ball stent (e.g. convex mesh, braid, matrix, or frame) can be radially-compressed (and longitudinally-elongated) for delivery through a catheter into an aneurysm sac and then radially-expanded (and longitudinally-shortened) within the aneurysm sac. In an example, a ball stent (e.g. convex mesh, braid, matrix, or frame) can self-expand radially after exiting a catheter. In an example, a ball stent (e.g. convex mesh, braid, matrix, or frame) can be expanded-radially by pulling, pushing, and/or rotating a wire or filament which is connected to it. In an example, a ball stent (e.g. convex mesh, braid, matrix, or frame) can be expanded-radially by the application of electrical energy.

In an example, a ball stent (e.g. convex mesh, braid, matrix, or frame) can be compressed and/or proximally-inverted by pulling a wire or filament which is connected to (a distal portion of) it. In an example, a ball stent (e.g. convex mesh, braid, matrix, or frame) can be compressed and/or proximally-inverted by pressure from (the accumulation of) embolic pieces (or flowable material) inserted between the ball stent (e.g. convex mesh, braid, matrix, or frame) and the dome of an aneurysm sac. Relevant variations discussed elsewhere in this disclosure or in priority-linked disclosures can also be applied to this example.

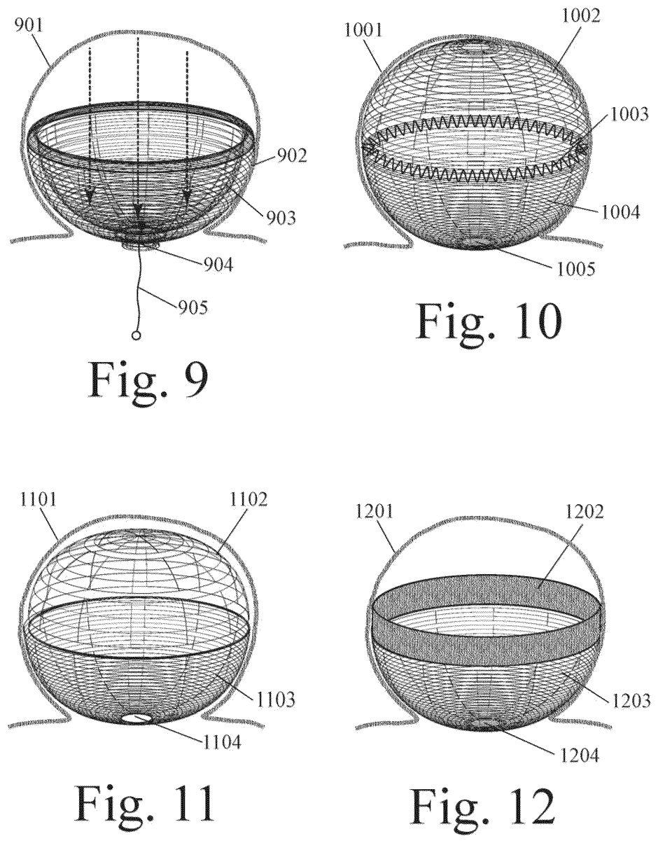

FIG. 9 shows a step in the formation of a two-layer distally-concave intrasaccular aneurysm occlusion device by proximally compressing and inverting a ball stent (e.g. a convex mesh, braid, matrix, or frame) within an aneurysm sac 901 into a two-layer distally-concave neck bridge with a proximal layer 902 and a distal layer 903 by pulling a longitudinal member (e.g. wire, cord, suture, thread, or filament) 905 which is connected to the distal layer. In this example, pulling the longitudinal member proximally compresses and inverts a distal portion (e.g. the distal half) of a ball stent into a concavity of a proximal portion (e.g. the proximal half) of the ball stent. In this example, there are openings 904 in the proximal and distal layers through which embolic pieces (or flowable material) can be inserted into the aneurysm sac.

In an example, the proximal portion of the ball stent can be held in place by a second longitudinal member (e.g. second wire), by attachment to a catheter, or another holding mechanism. This can prevent the entire device from being pulled out of the aneurysm sac when the longitudinal member attached to the distal portion is pulled in a proximal direction. In another example, a longitudinal member which is attached to a distal portion of the stent ball can be helically threaded and the stent ball can have corresponding helical threads, wherein rotation of the longitudinal member draws the distal portion of the stent ball closer to the proximal portion of the stent ball. In an example, one or more longitudinal members (e.g. wires) can be detached from the device (e.g. by the application of electrical energy) after the device has been collapsed from a ball shape to a bowl shape. In another example, a MEMS actuator can be used to draw the distal portion of the stent ball closer to the proximal portion of the stent ball. Relevant variations discussed elsewhere in this disclosure or in priority-linked disclosures can also be applied to this example.

FIG. 10 shows an example of an intrasaccular aneurysm occlusion device comprising: a proximal portion (e.g. proximal half) 1004 of a ball stent (e.g. convex mesh, stent, matrix, or frame) in an aneurysm sac 1001; a distal portion (e.g. distal half) 1002 of the ball stent (e.g. convex mesh, stent, matrix, or frame) in the aneurysm sac; an attachment mechanism 1003 which connects the proximal and distal portions; and an opening 1005 in a proximal surface of the proximal portion through which embolic pieces (or flowable material) are inserted into the convex member.

One advantage of this design is that it can make it easier to proximally collapse and invert a single-layer ball stent into a two-layer bowl-shaped neck bridge, especially if the structure is more flexible around the circumference where the two halves meet. Another potential advantage of this design is that the proximal and distal halves can be made with different materials and/or have different attributes. For example, the material or structure of the distal half can be more porous, more permeable, more flexible, more elastic, lower durometer, and/or more compliant than that of the proximal half.

In an example, forming a mesh ball, braid ball, or ball stent whose proximal and distal portions have different attributes (e.g. different levels of porosity, permeability, flexibility, compliance, durometer, and/or elasticity) can enable the ball to better fill and occlude the aneurysm sac. In an example, forming a mesh ball, braid ball, or ball stent whose proximal and distal portions have different attributes (e.g. different levels of porosity, permeability, flexibility, compliance, durometer, and/or elasticity) can also make it easier to compress and/or proximally invert a device from a ball shape to a bowl shape. In an example, a coating can be applied to a proximal portion, but not a distal portion, in order to create different levels of porosity, permeability, flexibility, compliance, durometer, and/or elasticity in proximal vs. distal portions of a ball stent.

In an example, an attachment mechanism can comprise wire which connects the proximal and distal portions. In an example, an attachment mechanism can comprise a polymer suture, thread, cord, or filament which connects the proximal and distal portions. In an example, proximal and distal portions can be sewn, braided, or woven together. In an example, an attachment mechanism can comprise adhesive material which connects the proximal and distal portions. In an example, proximal and distal portions can be glued together. In an example, proximal and distal portions can be melted and/or welded together. In an example, an attachment mechanism can comprise hooks, clasps, clips, teeth, loops, or snaps. In an example, hooks, clasps, clips, teeth, loops, or snaps on a proximal portion can connect with (e.g. interdigitate with) hooks, clasps, clips, teeth, loops, or snaps on a distal portion. Relevant variations discussed elsewhere in this disclosure or in priority-linked disclosures can also be applied to this example.

FIG. 11 shows an example of an intrasaccular aneurysm occlusion device comprising: a proximal portion (e.g. proximal half) 1103 of a ball stent (e.g. convex mesh, stent, matrix, or frame) in an aneurysm sac 1101; a distal portion (e.g. distal half) 1102 of the ball stent (e.g. convex mesh, stent, matrix, or frame) in the aneurysm sac, wherein the distal portion is made with different material and/or a different structure than the proximal portion; and an opening 1104 in a proximal surface of the proximal portion through which embolic pieces (or flowable material) are inserted into the convex member.

In an example, a proximal portion of a ball stent (e.g. convex mesh, braid, weave, matrix, or frame) can be made from one or more metals and a distal portion of the ball stent can be made from one or more polymers. In an example, the distal portion can be less dense than the proximal portion. In an example, the distal portion can be made with lower durometer material than that of the proximal portion. In an example, the distal portion can be more compliant than the proximal portion. In an example, the distal portion can be more elastic than the proximal portion. In an example, the distal portion can be more flexible than the proximal portion. In an example, the distal portion can be more permeable than the proximal portion. In an example, the distal portion can be more porous than the proximal portion. In an example, the distal portion can be thinner than the proximal portion. In an example, the distal portion can have larger pores, holes, and/or openings than the proximal portion. In an example, the distal portion can have more pores, holes, and/or openings than the proximal portion.

In an example, proximal and distal portions of a ball stent (e.g. convex mesh, braid, weave, matrix, or frame) can be the same size, comprising proximal and distal halves of the ball stent. In an example, proximal and distal portions of a ball stent (e.g. convex mesh, braid, weave, matrix, or frame) be different sizes, wherein the distal portion is larger than the proximal portion. In an example, proximal and distal portions of a ball stent (e.g. convex mesh, braid, weave, matrix, or frame) be different sizes, wherein the distal portion is 10% to 30% larger than the proximal portion. In an example, proximal and distal portions of a ball stent (e.g. convex mesh, braid, weave, matrix, or frame) be different sizes, wherein the proximal portion is 10% to 30% larger than the distal portion. In an example, proximal and distal portions of a ball stent (e.g. convex mesh, braid, weave, matrix, or frame) be different sizes, wherein the distal portion is 25% to 50% larger than the proximal portion. In an example, proximal and distal portions of a ball stent (e.g. convex mesh, braid, weave, matrix, or frame) be different sizes, wherein the proximal portion is 25% to 50% larger than the distal portion.

In an example, proximal and distal portions of a ball stent (e.g. convex mesh, braid, weave, matrix, or frame) can have different attributes because they are made separately and then attached together. In another example, proximal and distal portions of a ball stent (e.g. convex mesh, braid, weave, matrix, or frame) can have different attributes because a coating is applied to only one of the two portions. Relevant variations discussed elsewhere in this disclosure or in priority-linked disclosures can also be applied to this example.

FIG. 12 shows an example of an intrasaccular aneurysm occlusion device comprising: a distally-concave (e.g. bowl-shaped) neck bridge 1203 which is configured to be placed in aneurysm sac 1201, wherein there is an opening 1204 in the proximal surface of the neck bridge through which embolic pieces (or flowable material) can be inserted into the aneurysm sac; and a resilient annular band 1202 around the rim (e.g. the distal circumference) of the neck bridge, wherein the resilient annular band is stronger, thicker, less flexible, less compliant, and/or less elastic than the neck bridge, and wherein the annular band engages (e.g. presses against) the side walls of the aneurysm sac to hold the neck bridge in place. In an example, the annular band can be configured to span the aneurysm sac at its widest circumference. In an example, the annular band can have a larger circumference than the rest of the neck bridge and protrude out to engage the side walls of the aneurysm sac. Relevant variations discussed elsewhere in this disclosure or in priority-linked disclosures can also be applied to this example.

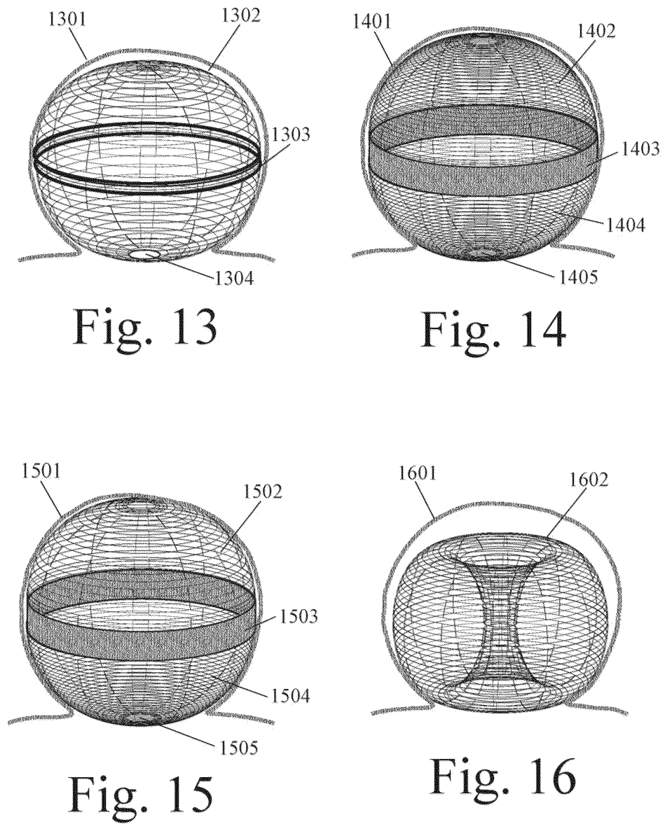

FIG. 13 shows an example of an intrasaccular aneurysm occlusion device comprising: a ball stent (e.g. convex mesh, braid, weave, matrix, or frame) 1302 which is configured to be placed in aneurysm sac 1301, wherein there is an opening 1304 in the proximal surface of the neck bridge through which embolic pieces (or flowable material) can be inserted into the ball stent; and a resilient annular band 1303 around a circumference of the ball stent, wherein the resilient annular band is stronger, thicker, less flexible, less compliant, and/or less elastic than the ball stent, and wherein the annular band engages (e.g. presses against) the side walls of the aneurysm sac to hold the ball stent in place. Goodness, gracious, great balls of wire. In an example, the annular band can be configured to span the ball stent at its widest circumference. Relevant variations discussed elsewhere in this disclosure or in priority-linked disclosures can also be applied to this example.

FIG. 14 shows an example of an intrasaccular aneurysm occlusion device comprising: a proximal portion (e.g. proximal half) 1404 of a ball stent (e.g. convex mesh, stent, matrix, or frame) in an aneurysm sac 1401; a distal portion (e.g. distal half) 1402 of the ball stent; an annular ring 1403 between (e.g. connecting) the proximal and distal portions; and an opening 1405 in a proximal surface of the proximal portion through which embolic pieces (or flowable material) are inserted into the ball stent.

In an example, an annular ring can span the equator (e.g. widest circumference) of a ball stent. In an example, the width of an annular ring can be between 5% and 20% of the longitudinal circumference of a ball stent. In an example, proximal and distal portions can be the same size. In another example a distal portion can be larger than a proximal portion. In another example a distal portion can be 20% to 50% larger than a proximal portion. In another example a proximal portion can be larger than a distal portion. In another example a proximal portion can be 20% to 50% larger than a distal portion. In an example, the annular band can have a larger circumference than the rest of the ball stent (e.g. somewhat like the rings of Saturn) and protrude out to engage the side walls of the aneurysm sac.

In an example, an annular ring can be more flexible or compliant than the proximal and distal portions of a ball stent. This can make it easier to proximally collapse and invert a single-layer ball stent into a two-layer bowl-shaped neck bridge. In another example, an annular ring can be less flexible or compliant than the proximal and distal portions of a ball stent. This can help to engage the side walls of the aneurysm sac and keep the stent ball within the aneurysm sac. In an example, proximal and distal portions can be made with different materials and/or have different attributes. For example, the material or structure of the distal portion can be more porous, more permeable, more flexible, more elastic, lower durometer, and/or more compliant than that of the proximal portion. Relevant variations discussed elsewhere in this disclosure or in priority-linked disclosures can also be applied to this example.

FIG. 15 shows an example of an intrasaccular aneurysm occlusion device comprising: a proximal portion (e.g. proximal half) 1504 of a ball stent (e.g. convex mesh, stent, matrix, or frame) in an aneurysm sac 1501; a distal portion (e.g. distal half) 1502 of the ball stent, wherein the distal portion is made with a different material and/or has a different structure than the proximal portion; an annular ring 1503 between (e.g. connecting) the proximal and distal portions; and an opening 1534025 in a proximal surface of the proximal portion through which embolic pieces (or flowable material) are inserted into the ball stent. For example, the material or structure of the distal portion can be more porous, more permeable, more flexible, more elastic, lower durometer, and/or more compliant than that of the proximal portion.

In an example, an annular ring can span the equator (e.g. widest circumference) of a ball stent. In an example, the width of an annular ring can be between 5% and 20% of the longitudinal circumference of a ball stent. In an example, proximal and distal portions can be the same size. In another example a distal portion can be larger than a proximal portion. In another example a distal portion can be 20% to 50% larger than a proximal portion. In another example a proximal portion can be larger than a distal portion. In another example a proximal portion can be 20% to 50% larger than a distal portion. In an example, the annular band can have a larger circumference than the rest of the ball stent (e.g. somewhat like the rings of Saturn) and protrude out to engage the side walls of the aneurysm sac.

In an example, an annular ring can be more flexible or compliant than the proximal and distal portions of a ball stent. This can make it easier to proximally collapse and invert a single-layer ball stent into a two-layer bowl-shaped neck bridge. In another example, an annular ring can be less flexible or compliant than the proximal and distal portions of a ball stent. This can help to engage the side walls of the aneurysm sac and keep the stent ball within the aneurysm sac. Relevant variations discussed elsewhere in this disclosure or in priority-linked disclosures can also be applied to this example.

FIG. 16 shows an example of an intrasaccular aneurysm occlusion device comprising: a torus-shaped mesh and/or stent 1602 which is configured to be inserted into and then radially-expanded within an aneurysm sac 1601, wherein a post-expansion diameter of the torus-shaped mesh and/or stent is configured to be greater than a diameter of the aneurysm neck.

In an example, a torus can be modeled as the revolution of a convex shape (e.g. circle or ellipse) around an axis which is coplanar with the shape, but outside the shape. In this example, the torus shape of the neck bridge can be modeled by revolving an ellipse (or oval) around an axis which is: coplanar with the ellipse (or oval), outside the ellipse (or oval), and parallel to the longitudinal axis of the ellipse (or oval). In an alternative example, a torus shape of a neck bridge can be modeled by revolving an ellipse (or oval) around an axis which is: coplanar with the ellipse (or oval), outside the ellipse (or oval), and perpendicular to the longitudinal axis of the ellipse (or oval). In an alternative example, a torus shape of a neck bridge can be modeled by revolving a circle around an axis which is coplanar with the circle and outside the circle. In an example, embolic pieces (or flowable material) can be inserted through the central-axial lumen of the torus-shaped mesh and/or stent into the aneurysm sac between the torus and the dome of the sac. Relevant variations discussed elsewhere in this disclosure or in priority-linked disclosures can also be applied to this example.

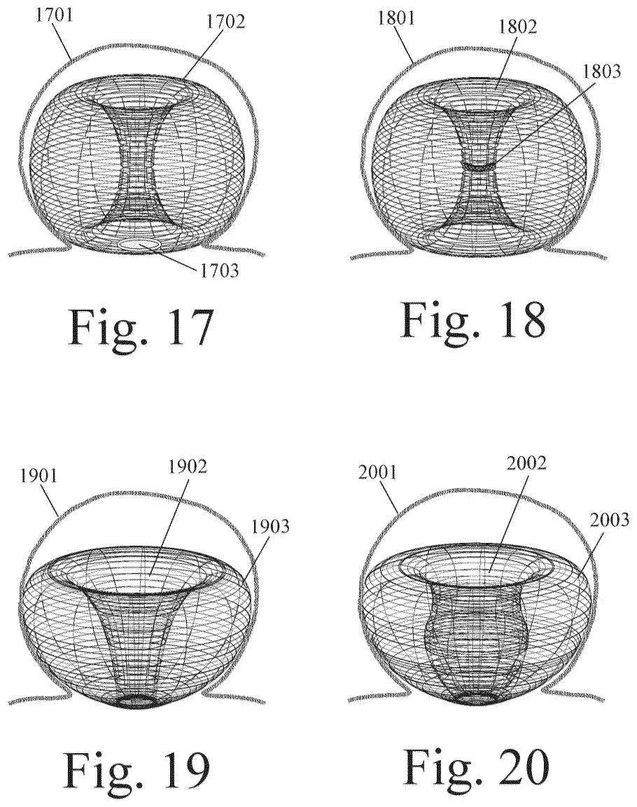

FIG. 17 shows an example of an intrasaccular aneurysm occlusion device like the one in FIG. 16 except that it includes an opening in the torus-shaped neck bridge through which embolic pieces (or flowable material) can be inserted into the interior of the torus. FIG. 17 shows an example of an intrasaccular aneurysm occlusion device comprising: a torus-shaped mesh and/or stent 1702 which is configured to be inserted into and then radially-expanded within an aneurysm sac 1701, wherein a post-expansion diameter of the torus-shaped mesh and/or stent is configured to be greater than a diameter of the aneurysm neck, and wherein there is an opening 1703 in a proximal surface of the torus-shaped mesh through which embolic pieces (or flowable material) can be inserted into the interior of the torus-shaped mesh. Relevant variations discussed elsewhere in this disclosure or in priority-linked disclosures can also be applied to this example.

FIG. 18 shows another example of an intrasaccular aneurysm occlusion device comprising: a torus-shaped mesh and/or stent 1802 which is configured to be inserted into and then radially-expanded within an aneurysm sac 1801, wherein a post-expansion diameter of the torus-shaped mesh and/or stent is configured to be greater than a diameter of the aneurysm neck. In this example, the torus-shaped mesh is formed by inverting the two ends of a tubular mesh and then attaching them together within the tube via attachment mechanism 1803. In an example, an attachment mechanism can be an annular band which attaches the inverted ends of a tubular mesh to each other. In an example, an attachment mechanism can be a wire, suture, or cord which attaches the inverted ends of a tubular mesh to each other. Relevant variations discussed elsewhere in this disclosure or in priority-linked disclosures can also be applied to this example.

FIG. 19 shows an example of an intrasaccular aneurysm occlusion device comprising: a torus-shaped mesh and/or stent 1903 which is configured to be inserted into and then radially-expanded within an aneurysm sac 1901, wherein a post-expansion diameter of the torus-shaped mesh and/or stent is configured to be greater than a diameter of the aneurysm neck, and wherein the torus shape can be modeled as the revolution of a convex shape with a single vertex (e.g. a tear-drop shape or flower-petal shape) around an axis which is coplanar with the convex shape and outside the convex shape. In this example, (an extension of) the axis around which the convex shape is revolved intersects (an extension of) the longitudinal axis of the convex shape at an oblique angle. In this example, a central axial lumen 1902 of the torus has a funnel, frustal, or hyperbolic shape and the diameter of this lumen increases in a proximal-to-distal direction. A convex tear-drop or flower-petal shape can be more precisely-described as a convex shape with a longitudinal axis, wherein a first end of the shape (at a first end of the longitudinal axis) comprises a smooth inwardly-concave curve and a second send of the shape (at a second end of the longitudinal axis) comprises an angular vertex (with an angle between 45 and 90 degrees). Relevant variations discussed elsewhere in this disclosure or in priority-linked disclosures can also be applied to this example.

FIG. 20 shows an example of an intrasaccular aneurysm occlusion device comprising: a torus-shaped mesh and/or stent 2003 which is configured to be inserted into and then radially-expanded within an aneurysm sac 2001, wherein a post-expansion diameter of the torus-shaped mesh and/or stent is configured to be greater than a diameter of the aneurysm neck, and wherein the torus shape can be modeled as the revolution of a Yin or Yang shape, comma shape, and/or paisley shape around an axis which is coplanar with the shape and outside the shape. In this example, a central axial lumen 2002 of the torus has the shape of a vase with a bulging middle section. A Yin or Yang shape is the shape of one of the two interdigitating geometric components of a Yin-Yang symbol. A comma shape is the shape of a comma (e.g. such as in the “Engravers MT” type font). A paisley shape can be mathematically represented using a Joukowsky transform. Relevant variations discussed elsewhere in this disclosure or in priority-linked disclosures can also be applied to this example.

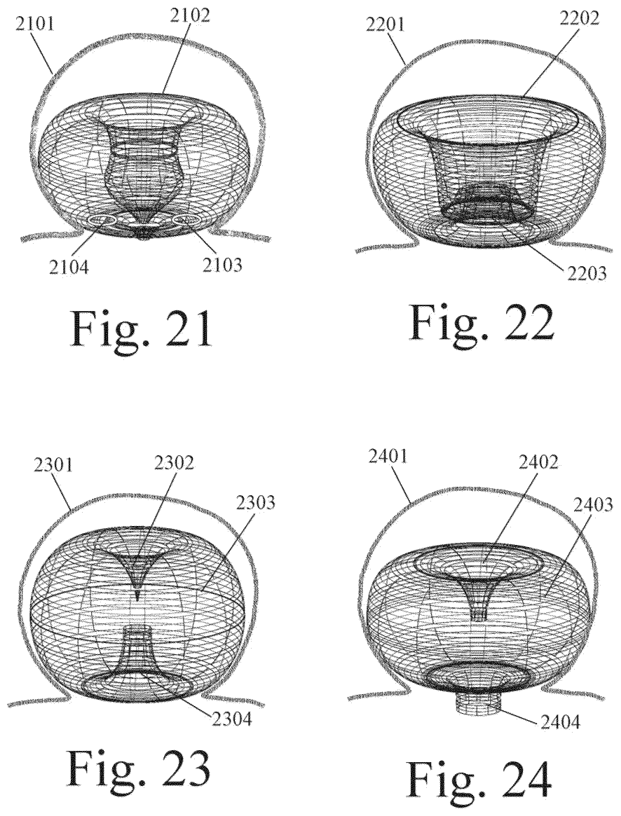

FIG. 21 shows an example of an intrasaccular aneurysm occlusion device like the one in FIG. 20 except that it includes one or more openings in the proximal surface of the torus-shaped mesh and/or stent through which embolic pieces (or flowable material) can be inserted into the interior of the mesh and/or stent. FIG. 21 shows an example of an intrasaccular aneurysm occlusion device comprising: a torus-shaped mesh and/or stent 2102 which is configured to be inserted into and then radially-expanded within an aneurysm sac 2101, wherein a post-expansion diameter of the torus-shaped mesh and/or stent is configured to be greater than a diameter of the aneurysm neck, wherein the torus shape can be modeled as the revolution of a Yin or Yang shape, comma shape, and/or paisley shape around an axis which is coplanar with the shape and outside the shape, and wherein there are one or more openings 2103 and 2104 in the proximal surface of the mesh and/or stent. Relevant variations discussed elsewhere in this disclosure or in priority-linked disclosures can also be applied to this example.

FIG. 22 shows an example of an intrasaccular aneurysm occlusion device like the one in FIG. 20 except that the proximal ends of the revolved shape (e.g. Yin or Yang shape, comma shape, or paisley shape) curve distally toward (e.g. into) the central axial lumen of the torus shape. FIG. 22 shows an example of an intrasaccular aneurysm occlusion device comprising: a torus-shaped mesh and/or stent 2202 which is configured to be inserted into and then radially-expanded within an aneurysm sac 2201, wherein a post-expansion diameter of the torus-shaped mesh and/or stent is configured to be greater than a diameter of the aneurysm neck, wherein the torus shape can be modeled as the revolution of a Yin or Yang shape, comma shape, and/or paisley shape around an axis which is coplanar with the shape and outside the shape. In this example, the diameter of the central axial lumen 2203 of the torus increases in a proximal-to-distal direction. Relevant variations discussed elsewhere in this disclosure or in priority-linked disclosures can also be applied to this example.

FIG. 23 shows an example of an intrasaccular aneurysm occlusion device comprising: a partial-torus (e.g. apple-shaped) mesh and/or stent 2303 which is configured to be inserted into and then radially-expanded within an aneurysm sac 2301, wherein a post-expansion diameter of the partial-torus mesh and/or stent is configured to be greater than a diameter of the aneurysm neck, wherein the distal surface of the partial-torus shape has a central proximally-pointing funnel and/or hyperbolic recession (e.g. local concavity) 2302, wherein the proximal surface of the partial-torus shape has a central distally-pointing funnel, frustal, and/or hyperbolic recession (e.g. local concavity) 2304, and wherein the central proximally-pointing funnel and/or hyperbolic recession and the central distally-pointing funnel, frustal, and/or hyperbolic recession do not connect with each other. In this example, the proximally-pointing funnel and/or hyperbolic recession is smaller (e.g. having a smaller diameter and/or length) than the distally-pointing funnel, frustal, and/or hyperbolic recession. Relevant variations discussed elsewhere in this disclosure or in priority-linked disclosures can also be applied to this example.

FIG. 24 shows an example of an intrasaccular aneurysm occlusion device comprising: a mesh and/or stent 2403 which is configured to be inserted into and then radially-expanded within an aneurysm sac 2401, wherein a post-expansion diameter of the mesh and/or stent is configured to be greater than a diameter of the aneurysm neck, wherein mesh and/or stent has a shape which can be modeling by revolving a question mark (without the dot portion) around an axis which is coplanar with the question mark and outside the concavity of the question mark. Revolution of the upper portion of the question mark creates a central proximally-pointing funnel, frustal, or hyperbolic shaped indentation 2402 in the distal surface of the mesh and/or stent. Revolution of the lower portion of the question mark creates a central proximally-pointing funnel, frustal, or hyperbolic shaped protrusion 2404 in the proximal surface of the mesh and/or stent. Relevant variations discussed elsewhere in this disclosure or in priority-linked disclosures can also be applied to this example.

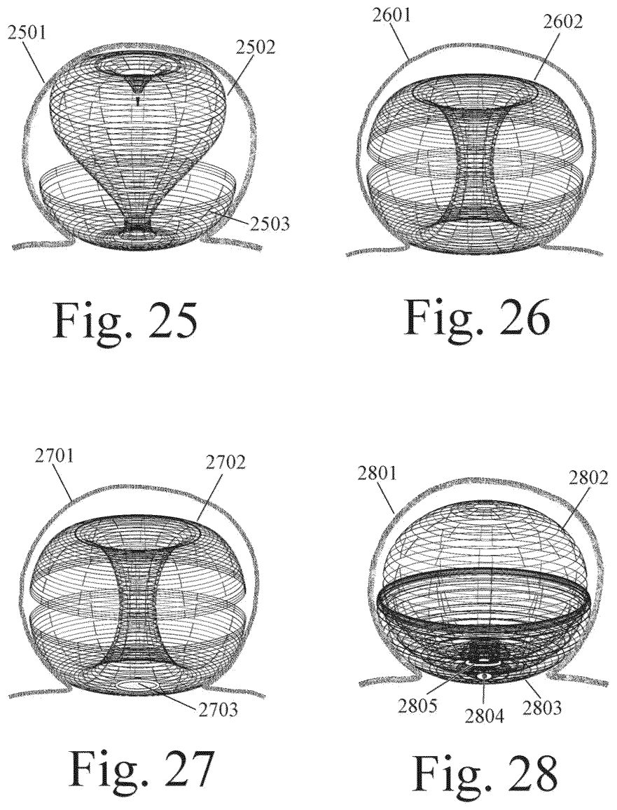

FIG. 25 shows an example of an intrasaccular aneurysm occlusion device comprising: a mesh and/or stent which is configured to be inserted into and then radially-expanded within an aneurysm sac 2501, wherein a post-expansion diameter of the mesh and/or stent is configured to be greater than a diameter of the aneurysm neck, wherein the mesh and/or stent further comprises a distal portion 2502 and a proximal portion 2503, wherein the distal portion has a 3D heart (e.g. revolution of a cardioid) shape, and wherein the proximal portion has a bowl (e.g. hemispherical) shape.

In an example, the distal portion can have a heart-shaped (stylized heart, not biologic heart) longitudinal cross-section. In an example, the distal portion can be modeled by revolving a longitudinal half of a (stylized, not biologic) heart shape around an axis which is coplanar with the heart shape, not within the heart shape, and parallel to the longitudinal axis of the heart shape. In an example, a distal surface of the distal portion can include a central proximally-pointing funnel, frustal, and/or hyperbolic indentation. In an example, the (cardioid-shaped) distal portion can extend from base of the (bowl-shaped) proximal portion to the dome of the aneurysm.

In an example, the proximal portion can form a neck bridge which is wider than the neck of the aneurysm sac. In an example, the proximal portion can have a bowl, hemispherical, or lower-partial-torus shape. In an example, the proximal and distal portions can be connected at the base of the proximal portion. In an example, the circumferences of proximal and distal portions can be the same size. In an example, the circumference of the distal portion can be 10% to 50% greater than the circumference of the proximal portion. In an example, the (proximal to distal) height of the (cardioid-shape) distal portion can be greater than the height of the (bowl-shaped) proximal portion. In an example, the (proximal to distal) height of the (cardioid-shape) distal portion can be 30% to 70% greater than the height of the (bowl-shaped) proximal portion.

In an example, the proximal and distal portions can be part of a continuous piece of material. In an example, the proximal and distal portions can be formed by multiple inversions of a tubular mesh. In an example, the proximal and distal portions can be formed by multiple inversions and radial constraints of a tubular mesh. In an example, the proximal and distal portions can be formed by inverting the distal end of a tubular mesh and everting the proximal end of the tubular mesh. In an example, the shape of the mesh and/or stent can be modeled by revolving an S-shape around an axis which is coplanar with the S-shape and outside the S-shape. In an example, the shape of the mesh and/or stent can be modeled by revolving an S-shape around an axis which is coplanar with the S-shape, outside the S-shape, and parallel to the longitudinal axis of the S-shape. Relevant variations discussed elsewhere in this disclosure or in priority-linked disclosures can also be applied to this example.

FIG. 26 shows an example of an intrasaccular aneurysm occlusion device comprising: a partial-torus mesh and/or stent 2602 which is configured to be inserted into and then radially-expanded within an aneurysm sac 2601; wherein a post-expansion diameter of the partial-torus mesh and/or stent is configured to be greater than a diameter of the aneurysm neck; wherein the shape of the partial-torus mesh and/or stent can be modeled by rotating a convex shape (e.g. circle, ellipse, or oval) with a missing perimeter section around an axis which is coplanar with the convex shape and outside the convex shape; wherein the missing perimeter section is on the opposite side of the convex shape from a side closest to the axis; and wherein the missing perimeter section comprises between 5% and 40% of the total perimeter of the convex shape.

In an example, this mesh or stent design could be figuratively called an “apple core” design because it looks a little like an apple core, although this is technically incorrect because, unlike a real apple, the central core is hollow and the proximal and distal ends overhang the core. In an example, the missing perimeter section can comprise 5% to 15% of total perimeter of the convex shape. In an example, the missing perimeter section can comprise 10% to 30% of total perimeter of the convex shape. In an example, the missing perimeter section can be centered on (a line which is perpendicular to) the longitudinal center of the convex shape. In an example, this mesh and/or stent can be formed by everting the proximal and distal ends of a tubular mesh. Relevant variations discussed elsewhere in this disclosure or in priority-linked disclosures can also be applied to this example.

FIG. 27 shows an example of an intrasaccular aneurysm occlusion device like the one in FIG. 26 except that it also includes an opening on the proximal surface of the mesh and/or stent (other than a central axial lumen) through which embolic pieces (or flowable material) can be inserted into the interior of the mesh and/or stent. FIG. 27 shows an example of an intrasaccular aneurysm occlusion device comprising: a partial-torus mesh and/or stent 2702 which is configured to be inserted into and then radially-expanded within an aneurysm sac 2701, wherein a post-expansion diameter of the partial-torus mesh and/or stent is configured to be greater than a diameter of the aneurysm neck, wherein the shape of the partial-torus mesh and/or stent can be modeled by rotating a convex shape (e.g. circle, ellipse, or oval) with a missing perimeter section around an axis which is coplanar with the convex shape and outside the convex shape, wherein the missing perimeter section is on the opposite side of the convex shape from a side closest to the axis, wherein the missing perimeter section comprises between 5% and 40% of the total perimeter of the convex shape, and wherein the mesh and/or stent further comprises an opening 2703 on the proximal surface of the mesh and/or stent through which embolic pieces (or flowable material) can be inserted into the interior of the mesh and/or stent. Relevant variations discussed elsewhere in this disclosure or in priority-linked disclosures can also be applied to this example.

FIG. 28 shows an example of an intrasaccular aneurysm occlusion device comprising: a proximal concave (e.g. bowl and/or hemisphere shaped) neck bridge 2803 which is configured to be inserted into and then radially-expanded within an aneurysm sac 2801, wherein a post-expansion diameter of the neck bridge and/or stent is configured to be greater than a diameter of the aneurysm neck; a flexible distal net or mesh 2802 which is configured to be between the neck bridge and the dome of the aneurysm sac; a proximal annular member (e.g. band or ring) 2805 which radially constrains a proximal portion of the neck bridge; and a proximal opening and/or valve 2804 through which embolic pieces (or flowable material) can be inserted through the neck bridge into the distal net or mesh.

In an example, a proximal concave neck bridge can have a bowl shape. In an example, a proximal concave neck bridge can have a hemispherical shape. In an example, a proximal concave neck bridge can have a partial-torus (e.g. lower-half of a torus) shape. In an example, a proximal concave neck bridge can have two layers (e.g. a proximal layer and a distal layer). In an example, a proximal concave neck bridge can have a proximal layer and a distal layer which are continuous with each other at a circumferential rim (e.g. fold line). In an example, a proximal concave neck bridge can be formed by compressing and inverting a single-layer globular mesh into a two-layer bowl-shaped mesh. In an example, a proximal concave neck bridge can be braided and/or woven from metal wires or tubes.

In an example, a flexible distal net or mesh can be more flexible, more elastic, thinner, and/or lower durometer than a proximal concave neck bridge. In an example, a flexible distal net or mesh can be more porous than a proximal concave neck bridge. In an example, a distal net or mesh can have a globular shape. In an example, a distal net or mesh can be expanded by the insertion of embolic pieces (or flowable material) into it through a proximal opening and/or valve. In an example, a distal net or mesh can be a polymer net or mesh. In an example, a distal net or mesh can be a honeycomb net or mesh (e.g. with a plurality of hexagonal pores or openings). In an example, a distal net or mesh can be made by braiding, weaving, 3D printing, or laser cutting.

In an example, a portion (e.g. between 25% and 75%) of a distal net or mesh can be nested inside the concavity of a proximal concave neck bridge. In an example, a distal net or mesh can be partially nested within the concavity of a proximal concave neck bridge. In another example, a proximal concave neck bridge can be inside a distal net or mesh. In an example, a proximal concave neck bridge and a distal net or mesh can be attached to each other by the proximal annular member. In an example, a proximal concave neck bridge and a distal net or mesh can be attached to each other before insertion into a catheter for delivery to an aneurysm sac. In an example, a proximal concave neck bridge and a distal net or mesh can be separately delivered through a catheter to an aneurysm sac and then attached to each other within the aneurysm sac. In an example, a distal net or mesh can be attached to the base (e.g. bottom) of a proximal concave neck bridge.

In an example, a proximal valve can be used (e.g. controlled by a device operator) to close an opening through a proximal concave neck bridge after embolic pieces (or flowable material) have been inserted through it into the distal net or mesh. In an example, a proximal valve and/or opening can be centrally located on the proximal surface of a proximal concave neck bridge. In an example, a proximal valve and/or opening can be through a central lumen of a proximal annular member which radially constrains a proximal concave neck bridge.

In an example, a proximal valve can be closed by the application of electrical energy, by pulling or pushing a wire, by rotating a wire, by pushing a plug, or by pumping congealing material. In an example, embolic pieces (or flowable material) can be selected from the group consisting of: micro-sponges; compressible micro-balls; embolic beads; pieces of foam; pieces of hydrogel; string-of-pearls embolic strands (e.g. embolic pieces connected by flexible longitudinal filaments, wires, coils, or threads); embolic coils; embolic ribbons; and liquid embolic material (e.g. flowable liquid or gel that congeals within an aneurysm sac). Relevant variations discussed elsewhere in this disclosure or in priority-linked disclosures can also be applied to this example.

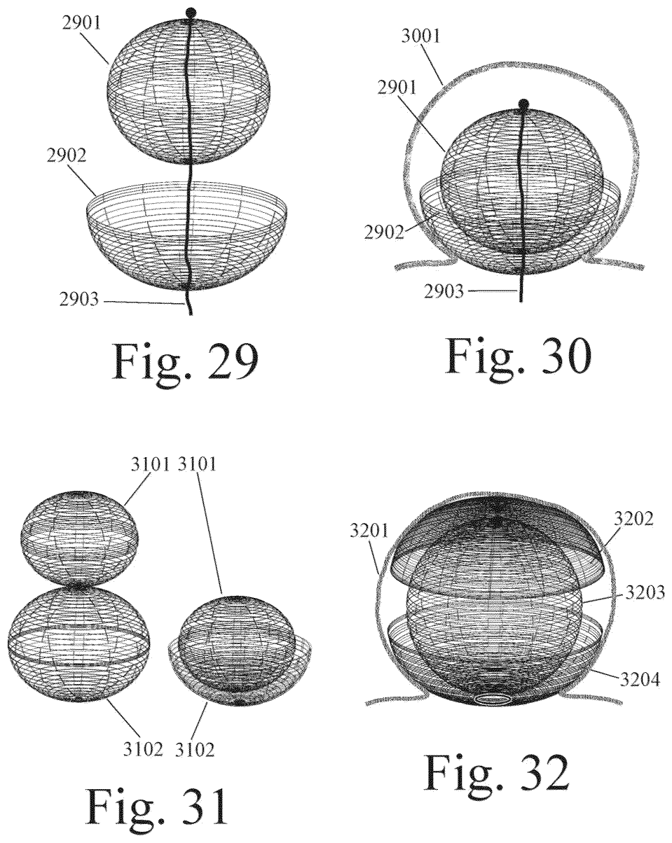

FIGS. 29 and 30 show an example of an intrasaccular aneurysm occlusion device comprising: a proximal concave (e.g. bowl and/or hemisphere shaped) neck bridge 2902 which is configured to be inserted into and then radially-expanded within an aneurysm sac 3001, wherein a post-expansion diameter of the neck bridge and/or stent is configured to be greater than a diameter of the aneurysm neck; a distal globular (e.g. ball and/or sphere shaped) net or mesh 2901 which is configured to be inserted into and then radially-expanded within the aneurysm sac; and a longitudinal member (e.g. wire, suture, or cord) 2903 which is connected to the globular net or mesh, wherein pulling the longitudinal member pulls the globular net or mesh toward the neck bridge so that the globular net or mesh becomes (partially) nested within the concavity of the neck bridge.

FIG. 29 shows this device at a first time before the longitudinal member has been pulled, wherein the globular mesh or net is not nested within a concavity of the neck bridge. FIG. 30 shows this device at a second time after the longitudinal member has been pulled, wherein the globular mesh or net is partially nested within a concavity of the neck bridge. In an example, the longitudinal member can pass (e.g. slide) through an opening in the neck bridge so that pulling the longitudinal member moves the globular mesh or net, but not the neck bridge. Relevant variations discussed elsewhere in this disclosure or in priority-linked disclosures can also be applied to this example.

FIG. 31 shows an example of an intrasaccular aneurysm occlusion device comprising: a proximal stent (e.g. mesh or frame) 3102; and a distal stent (e.g. mesh or frame) 3101; wherein the device has a first configuration in which the proximal stent is globular, the proximal stent has one layer, and the distal stent is not nested within the proximal stent; and wherein the device has a second configuration in which the proximal stent is concave, the proximal stent has two layers, and the distal stent is at least partially nested within a concavity of the proximal stent. The left side of FIG. 31 shows this device in the first configuration. The right side of FIG. 31 shows this device in the second configuration. In an example, the proximal stent can be compressed and inverted from a two-layer concave (e.g. ball) shape to a one-layer concave (e.g. bowl) shape in the transition from the first configuration to the second configuration. Relevant variations discussed elsewhere in this disclosure or in priority-linked disclosures can also be applied to this example.

FIG. 32 shows an example of an intrasaccular aneurysm occlusion device comprising: a proximal concave (e.g. bowl-shaped) stent (e.g. mesh or frame) 3204; a distal concave (e.g. inverted-bowl-shaped) stent (e.g. mesh or frame) 3202; and a middle convex (e.g. globular) stent (e.g. mesh or frame) 3203 between the proximal concave stent and the distal concave stent, wherein the middle convex stent is partially nested within a concavity of the proximal concave stent and partially nested within a concavity of the distal concave stent; wherein the proximal concave, distal concave, and middle convex stents are configured to be inserted into and expanded within an aneurysm sac 3201. Relevant variations discussed elsewhere in this disclosure or in priority-linked disclosures can also be applied to this example.

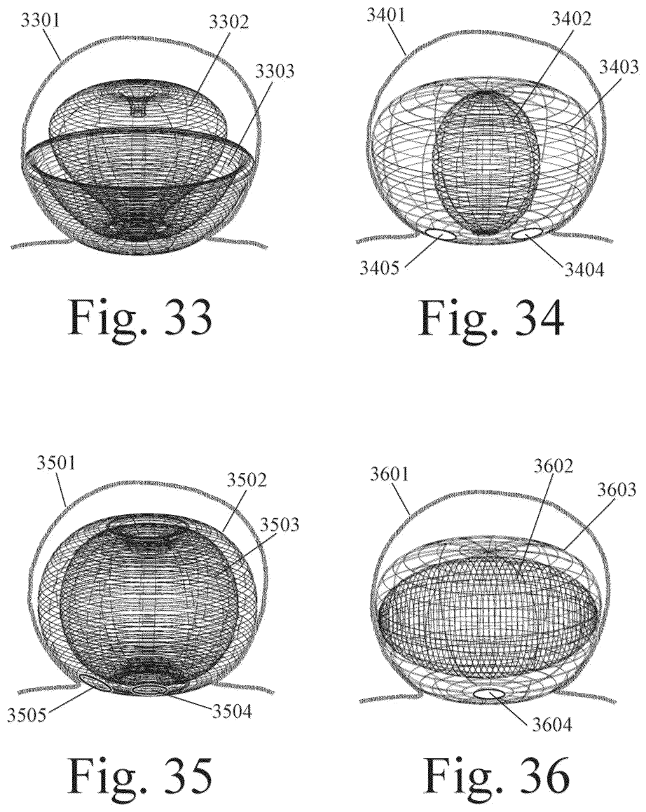

FIG. 33 shows an example of an intrasaccular aneurysm occlusion device comprising: a mesh and/or stent which is configured to be inserted into and then radially-expanded within an aneurysm sac 3301, wherein a post-expansion diameter of the mesh and/or stent is configured to be greater than a diameter of the aneurysm neck, wherein the mesh and/or stent further comprises a bulbous distal portion 3302 and a concave proximal portion 3303.

In an example, a bulbous distal portion can have a generally-convex shape except for a central funnel and/or indentation on its distal surface. In an example, a bulbous distal portion can have a generally-convex shape except for a central proximally-pointing funnel and/or indentation on its distal surface. In an example, a bulbous distal portion can be radially-symmetric around its central longitudinal axis. In an example, a bulbous distal portion can extend in a distal direction from the base of a concave proximal portion. In an example, a bulbous distal portion can span from the base of a concave proximal portion to the dome of an aneurysm sac.

In an example, a concave proximal portion can form a neck bridge which is wider than the neck of the aneurysm sac. In an example, a concave proximal portion can have a bowl, hemispherical, or lower-partial-torus shape. In an example, a concave proximal portion can have two layers. In an example, proximal and distal portions can be connected at the base of the proximal portion. In an example, the circumference of the proximal portion can be 10% to 50% greater than the circumference of the distal portion.

In an example, proximal and distal portions can be formed from a continuous piece of material. In an example, proximal and distal portions can be formed by multiple inversions of a tubular mesh. In an example, proximal and distal portions can be formed by multiple inversions and radial constraints of a tubular mesh. In an example, proximal and distal portions can be formed by inverting the distal end of a tubular mesh and everting the proximal end of the tubular mesh. Relevant variations discussed elsewhere in this disclosure or in priority-linked disclosures can also be applied to this example.

FIG. 34 shows an example of an intrasaccular aneurysm occlusion device comprising: an outer flexible mesh or net 3403 which is configured to be inserted into an aneurysm sac 3401, wherein there are one or more openings 3404 and 3405 in the proximal surface of the outer flexible mesh or net through which embolic pieces (or flowable material) can be inserted into the outer flexible mesh or net; and an inner convex stent 3402 which is inside the outer flexible mesh or net. In an example, an inner convex stent can have a globular, spherical, and/or ellipsoidal shape. In an example, an inner convex stent can be less flexible, less elastic, and/or less compliant than an outer flexible mesh or net. In an example, an inner convex stent can be a metal mesh (or frame) and an outer mesh or net can be a polymer mesh or net. In an example, an inner convex stent can self-expand within an aneurysm sac. In an example, an outer mesh or net can be expanded within an aneurysm sac by the insertion of embolic pieces (or flowable material) through the one or more openings. Relevant variations discussed elsewhere in this disclosure or in priority-linked disclosures can also be applied to this example.

FIG. 35 shows an example of an intrasaccular aneurysm occlusion device which is like the example in FIG. 34 except that it also includes an opening in the inner convex stent through which embolic pieces (or flowable material) can be inserted into the inner convex stent. FIG. 35 shows an example of an intrasaccular aneurysm occlusion device comprising: an outer flexible mesh or net 3502 which is configured to be inserted into an aneurysm sac 3501; an opening 3505 in the outer flexible mesh or net through which embolic pieces (or flowable material) can be inserted into the outer flexible mesh or net; an inner convex stent 3503 which is inside the outer flexible mesh or net; and an opening 3504 in the inner convex stent through which embolic pieces (or flowable material) can be inserted into the inner convex stent. In an example, an inner convex stent can have a globular, spherical, and/or ellipsoidal shape. In an example, an inner convex stent can be less flexible, less elastic, and/or less compliant that the outer flexible net or mesh. Relevant variations discussed elsewhere in this disclosure or in priority-linked disclosures can also be applied to this example.

FIG. 36 shows an example of an intrasaccular aneurysm occlusion device which is like the example in FIG. 34 except that the inner convex stent spans a circumference of the aneurysm sac. FIG. 36 shows an example of an intrasaccular aneurysm occlusion device comprising: an outer flexible mesh or net 3603 which is configured to be inserted into an aneurysm sac 3601; an opening 3604 in the outer flexible mesh or net through which embolic pieces (or flowable material) can be inserted into the outer flexible mesh or net; and an inner convex stent 3602 which is inside the outer flexible mesh or net, wherein the inner convex stent spans a circumference of the aneurysm sac. In an example, an inner convex stent can have a globular, spherical, and/or ellipsoidal shape. In an example, an inner convex stent can span the greatest circumference of the aneurysm sac. In an example, an inner convex stent can be less flexible, less elastic, and/or less compliant than the outer flexible net or mesh. Relevant variations discussed elsewhere in this disclosure or in priority-linked disclosures can also be applied to this example.

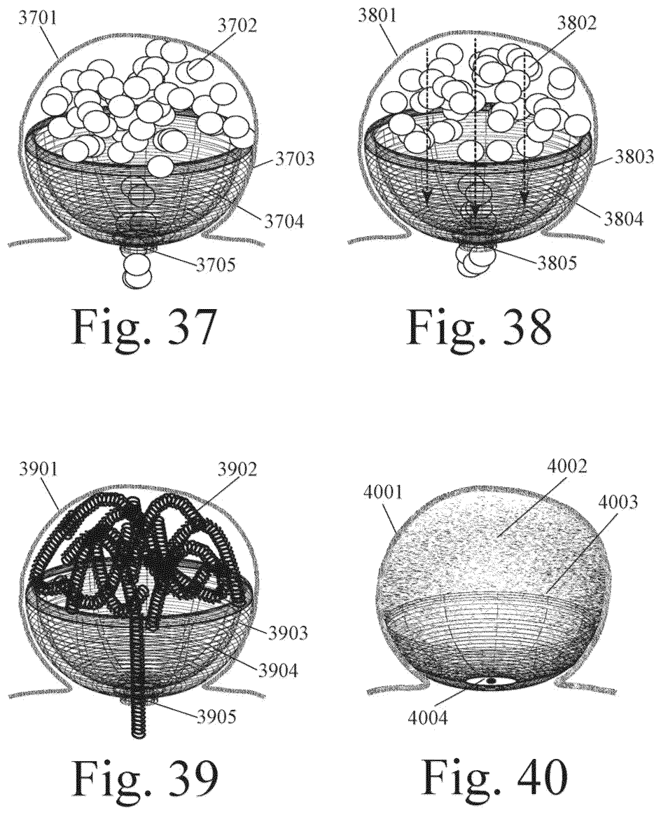

FIG. 37 shows an example of an intrasaccular aneurysm occlusion device comprising: a two-layer proximal distally-concave (e.g. bowl and/or hemisphere shaped) neck bridge which is configured to be inserted into and expanded within an aneurysm sac 3701, wherein the neck bridge further comprises a distal layer 3704 and a proximal layer 3703, and wherein there is a central opening 3705 in the neck bridge through which embolic pieces (e.g. microsponges, microbeads, hydrogel pieces, or foam pieces) 3702 are inserted into the aneurysm sac.

In an example, a two-layer proximal distally-concave neck bridge can have a bowl shape. In an example, a neck bridge can have shape which is a section (e.g. between 30% and 70%) of the perimeter of a sphere. In an example, a neck bridge can have a hemispherical shape. In an example, a neck bridge can have shape which is a section (e.g. between 30% and 70%) of the perimeter of an ellipsoid. In an example, a neck bridge can have a half-ellipsoidal shape. In an example, a neck bridge can have shape which is a section (e.g. between 30% and 70%) of the perimeter of an torus. In an example, a neck bridge can have a half-toroidal shape (e.g. the lower half like a cut bagel). In an example, a neck bridge can have an inverted-umbrella shape.

In an example, a two-layer proximal distally-concave neck bridge can be radially-compressed (and longitudinally-elongated) for delivery through a catheter to an aneurysm sac and then radially-expanded (and longitudinally-shortened) within the aneurysm sac. In an example, a neck bridge can self-expand radially after exiting a catheter. In an example, a neck bridge can be expanded-radially after exiting a catheter by a wire (or filament) connected to it which is pushed, pulled, or rotated. In an example, a neck bridge can be expanded-radially after exiting a catheter by the application of electrical energy.

In an example, a two-layer distally-concave neck bridge can comprise a mesh, braid, or stent. In an example, a neck bridge can comprise a woven or braided mesh, braid, or stent. In an example, a neck bridge can be made by 3D printing. In an example, a neck bridge can be made by laser cutting. In an example, a neck bridge can be made from metal wires, tubes, and/or coils. In an example, a neck bridge can be made from polymer strands, filaments, threads, or yarns. In an example, a neck bridge can be made from organic strands, filaments, threads, or yarns. In an example, a neck bridge can be made from metal(s), polymer(s), or a combination of metal(s) and polymer(s).

In an example, a two-layer distally-concave neck bridge can be formed by proximally compressing and inverting a convex (e.g. spherical) member into a concave (e.g. bowl-shaped) member. In an example, a neck bridge can comprise two layers (e.g. proximal and distal layers) which are continuous with each other at a distal (e.g. radial perimeter) fold. In an example, a neck bridge can comprise two layers (e.g. proximal and distal layers) which are connected to each other by a proximal annular member (e.g. band, ring, hub, twisted wire, zip tie, cord, clip, or washer). In an example, a proximal annular member can bind together the proximal ends of an inverted and/or folded mesh or net that forms a neck bridge. In an example, a neck bridge can comprise two layers (e.g. proximal and distal layers) which are connected to each other by two concentric proximal annular members (e.g. bands, rings, hubs, wires, cords, clips, or washers), wherein ends of the two layers are inserted between the two concentric proximal annual members.

In an example, there can be a uniform distance between proximal and distal layers of a distally-concave neck bridge. In an example, the distance between proximal and distal layers of a distally-concave neck bridge can increase with distance from the aneurysm neck. In an example, the distance between proximal and distal layers of a distally-concave neck bridge can increase and then decrease with distance from the aneurysm neck. In an example, the distance between proximal and distal layers of a distally-concave neck bridge can decrease with distance from the aneurysm neck. In an example, an interior space between proximal and distal layers in a neck bridge can be convex. In an example, the longitudinal cross-sectional shape of an interior space between proximal and distal layers can be elliptical, oval, comma shaped, paisley shaped, crescent shaped, or egg shaped.

In an example, a distally-concave neck bridge can comprise three layers, wherein the proximal and distal layers are portions of the same piece of material (e.g. the same mesh or braid) and a middle layer between the proximal and distal layers is a separate piece of material. In an example, the proximal and distal layers can be metal and the middle layer can be made from a polymer or organic-material. In an example, the middle layer can comprise a membrane, film, mesh, or filter. In an example, the middle layer can be made from hydrogel.

In an example, openings (e.g. holes, lumens, or tubes) in proximal and distal layers of a two-layer distally-concave neck bridge can be located at the proximal centers of these layers. In an example, openings (e.g. holes, lumens, or tubes) in proximal and distal layers of a neck bridge can be aligned with each other. In an example, an openings in proximal and distal layers can be through the interior (e.g. central lumen) of a proximal annular member (e.g. band or ring) which connects proximal portions of the proximal and distal layers of the concave member. In an example, openings in proximal and distal layers can be through the interior (e.g. central opening) of concentric proximal annular members (e.g. bands or rings) which connect portions of proximal and distal layers of the concave member.

In an example, a two-layer distally-concave neck bridge can comprise a mesh or net with a plurality of pores, holes, or gaps. In an example, these pores, holes, or gaps can be hexagonal. In an example, these pores, holes, or gaps can be triangular. In an example, these pores, holes, or gaps can be circular. In an example, embolic pieces which are inserted through a central opening in the neck bridge can be larger than (e.g. at least twice as large as) any of the pores, holes, or gaps in the mesh or net. In an example, a central proximal opening in the neck bridge through which embolic pieces are inserted can be larger than (e.g. at least twice as large as) any of the plurality of pores, holes, or gaps in the mesh or net. In an example, a device can comprise non-central openings in the proximal surface of a neck bridge through which embolic pieces (or flowable material) can be inserted into an aneurysm sac, wherein these two or more non-central openings are larger than (e.g. at least twice as large as) any of the (other) plurality of pores, holes, or gaps in the mesh or net.

In an example, a device can further comprise a closure mechanism (e.g. valve, plug, clip, or loop) which can be used (e.g. controlled by a device operator) to close an opening and/or valve after embolic pieces (or flowable material) have been inserted through the opening into the aneurysm sac. In an example, a closure mechanism can be activated by the application of electrical energy, by pulling or pushing a wire, by rotating a wire, by pushing a plug, or by pumping congealing material. In an example, embolic pieces which are inserted through an opening into an aneurysm sac can be selected from the group consisting of: micro-sponges; compressible micro-balls; embolic beads; pieces of foam; and pieces of hydrogel. Relevant variations discussed elsewhere in this disclosure or in priority-linked disclosures can also be applied to this example.

FIG. 38 shows an example of an intrasaccular aneurysm occlusion device like the example shown in FIG. 37 except the two-layer proximal distally-concave neck bridge is formed by proximally compressing and inverting a one-layer globular net or mesh within the aneurysm sac. FIG. 38 shows an example of an intrasaccular aneurysm occlusion device comprising: a two-layer proximal distally-concave (e.g. bowl and/or hemisphere shaped) neck bridge which is formed by proximally compressing and inverting a one-layer globular net or mesh in an aneurysm sac 3801, wherein the neck bridge after compression and inversion comprises a distal layer 3804 and a proximal layer 3803, and wherein there is a central opening 3805 in the neck bridge through which embolic pieces (e.g. microsponges, microbeads, hydrogel pieces, or foam pieces) 3802 are inserted into the aneurysm sac. In an example, the neck bridge can be compressed and inverted by pressure from the accumulation of embolic pieces (or flowable material) in the aneurysm sac between the neck bridge and the dome of the aneurysm. Relevant variations discussed elsewhere in this disclosure or in priority-linked disclosures can also be applied to this example.

FIG. 39 shows an example of an intrasaccular aneurysm occlusion device which is like the example shown in FIG. 37 except that embolic coils are inserted into the aneurysm sac. FIG. 39 shows an example of an intrasaccular aneurysm occlusion device comprising: a two-layer proximal distally-concave (e.g. bowl and/or hemisphere shaped) neck bridge which is configured to be inserted into and expanded within an aneurysm sac 3901, wherein the neck bridge further comprises a distal layer 3904 and a proximal layer 3903, and wherein there is a central opening 3905 in the neck bridge through which one or more embolic coils 3902 are inserted into the aneurysm sac. Relevant variations discussed elsewhere in this disclosure or in priority-linked disclosures can also be applied to this example.

FIG. 40 shows an example of an intrasaccular aneurysm occlusion device which is like the example shown in FIG. 37 except that an embolic flowable substance (e.g. congealing liquid or gel) is inserted through a valve in the neck bridge into the aneurysm sac. FIG. 40 shows an example of an intrasaccular aneurysm occlusion device comprising: a proximal distally-concave (e.g. bowl and/or hemisphere shaped) neck bridge 4003 which is configured to be inserted into and expanded within an aneurysm sac 4001, wherein there is a (central) valve 4004 in the neck bridge through which an embolic flowable substance (e.g. congealing liquid or gel) 4002 is inserted into the aneurysm sac. In an example, the valve can be a one-way valve which allows a flowable substance to be inserted through the neck bridge into the aneurysm sac, but not leak out. In an example, the valve can be a remotely opened or closed by the device operator (e.g. by the application of electrical energy or moving a wire). Relevant variations discussed elsewhere in this disclosure or in priority-linked disclosures can also be applied to this example.

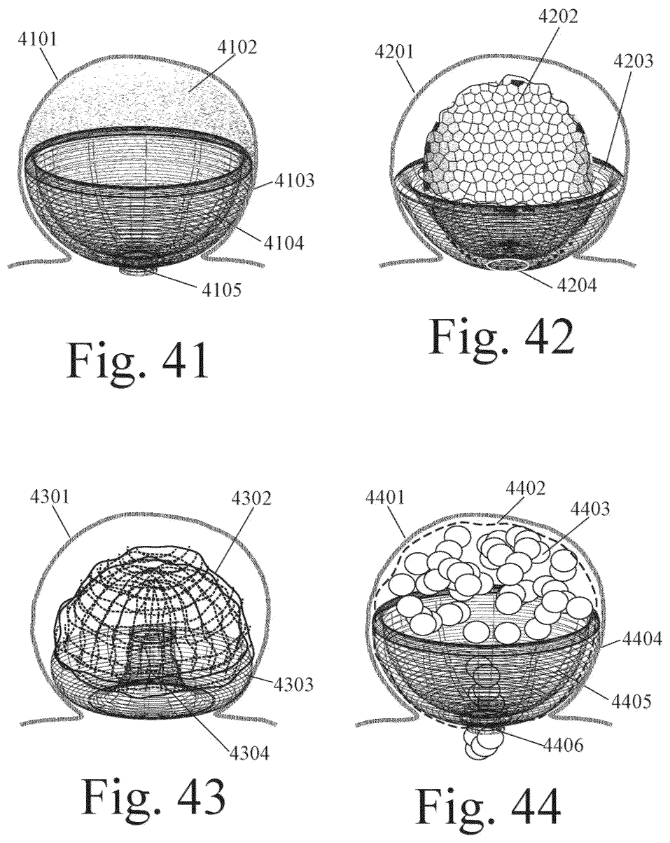

FIG. 41 shows an example of an intrasaccular aneurysm occlusion device comprising: a two-layer proximal distally-concave (e.g. bowl and/or hemisphere shaped) neck bridge which is configured to be inserted into and expanded within an aneurysm sac 4101, wherein the neck bridge further comprises a distal layer 4104 and a proximal layer 4103, and wherein there is a central proximal opening 4105 in the neck bridge through which an embolic flowable substance (e.g. congealing liquid or gel) 4102 is inserted into the aneurysm sac. Relevant variations discussed elsewhere in this disclosure or in priority-linked disclosures can also be applied to this example.

FIG. 42 shows an example of an intrasaccular aneurysm occlusion device comprising: a proximal distally-concave (e.g. bowl and/or hemisphere shaped) neck bridge 4203 which is configured to be inserted into and expanded within an aneurysm sac 4201; and a distal mesh or net 4202 which is at least partially nested within the concavity of the proximal distally-concave neck bridge; wherein embolic pieces (or flowable material) are inserted into the distal mesh or net through a central proximal opening 4204 in the neck bridge. In an example, the distal mesh or net can be more flexible, more elastic, more compliant, thinner, less dense, and/or more porous than the neck bridge. In an example, the distal mesh can be made from one or more polymers and the neck bridge can be made from one or more metals. In an example, the distal mesh or net can expand to conform to the distal walls (e.g. the dome) of even an irregularly-shaped aneurysm when embolic pieces (or flowable material) are inserted into it. In an example, the distal mesh or net can be a honeycomb mesh (e.g. having a plurality of hexagonal pores). Relevant variations discussed elsewhere in this disclosure or in priority-linked disclosures can also be applied to this example.

FIG. 43 shows an example of an intrasaccular aneurysm occlusion device comprising: a proximal partial-torus neck bridge 4303 which is configured to be inserted into and expanded within an aneurysm sac 4301; and a distal mesh or net 4302 between the neck bridge and the dome of the aneurysm sac; wherein embolic pieces (or flowable material) are inserted into the distal mesh or net through a central funnel-shaped lumen 4304 of the partial-torus neck bridge. In an example, a central funnel-shaped structure of a partial-torus neck bridge can extend farther in a distal direction than a circumferential rim of the partial-torus neck bridge. In an example, the distal mesh or net can be more flexible, more elastic, more compliant, thinner, less dense, and/or more porous than the neck bridge. In an example, the distal mesh or net can expand to conform to the distal walls (e.g. the dome) of even an irregularly-shaped aneurysm when embolic pieces (or flowable material) are inserted into it. Relevant variations discussed elsewhere in this disclosure or in priority-linked disclosures can also be applied to this example.

FIG. 44 shows an example of an intrasaccular aneurysm occlusion device comprising: a proximal two-layer distally-concave (e.g. bowl and/or hemisphere shaped) neck bridge which is configured to be inserted into and expanded within an aneurysm sac 4401, wherein the neck bridge further comprises a proximal layer 4404 and a distal layer 4405; a distal flexible net or mesh 4402 (at least partially) between the neck bridge and the dome of the aneurysm sac; and a central opening 4406 in the neck bridge through which embolic pieces (e.g. microsponges, microbeads, hydrogel pieces, or foam pieces) 4403 are inserted into the distal flexible net or mesh.

In an example, a two-layer distally-concave neck bridge can have a bowl shape. In an example, a neck bridge can have shape which is a section (e.g. between 30% and 70%) of the perimeter of a sphere. In an example, a neck bridge can have a hemispherical shape. In an example, a neck bridge can have shape which is a section (e.g. between 30% and 70%) of the perimeter of an ellipsoid. In an example, a neck bridge can have a half-ellipsoidal shape. In an example, a neck bridge can have shape which is a section (e.g. between 30% and 70%) of the perimeter of an torus. In an example, a neck bridge can have a half-toroidal shape (e.g. the lower half like a cut bagel). In an example, a neck bridge can have an inverted-umbrella shape.

In an example, a neck bridge can be radially-compressed (and longitudinally-elongated) for delivery through a catheter to an aneurysm sac and then radially-expanded (and longitudinally-shortened) within the aneurysm sac. In an example, a neck bridge can self-expand radially after exiting a catheter. In an example, a neck bridge can be expanded-radially after exiting a catheter by a wire (or filament) connected to it which is pushed, pulled, or rotated. In an example, a neck bridge can be expanded-radially after exiting a catheter by the application of electrical energy.

In an example, a neck bridge can comprise a mesh, braid, or stent. In an example, a neck bridge can comprise a woven or braided mesh, braid, or stent. In an example, a neck bridge can comprise a 3D printed mesh, braid, or stent. In an example, a neck bridge can comprise a laser-cut mesh, braid, or stent. In an example, a neck bridge can be made from metal wires, tubes, and/or coils. In an example, a neck bridge can be made from polymer strands, filaments, threads, or yarns. In an example, a neck bridge can be made from organic strands, filaments, threads, or yarns. In an example, a neck bridge can be made from metal(s), polymer(s), or a combination of metal(s) and polymer(s).

In an example, a two-layer distally-concave neck bridge can be formed by proximally compressing and inverting a convex (e.g. spherical) member into a concave (e.g. bowl-shaped) member. In an example, a neck bridge can comprise two layers (e.g. proximal and distal layers) which are continuous with each other at a distal (e.g. radial perimeter) fold. In an example, a neck bridge can comprise two layers (e.g. proximal and distal layers) which are connected to each other by a proximal annular member (e.g. band, ring, hub, twisted wire, zip tie, cord, clip, or washer). In an example, a proximal annular member can bind together the proximal ends of an inverted and/or folded mesh or net that forms a neck bridge. In an example, a neck bridge can comprise two layers (e.g. proximal and distal layers) which are connected to each other by two concentric proximal annular members (e.g. bands, rings, hubs, wires, cords, clips, or washers), wherein ends of the two layers are inserted between the two concentric proximal annual members.

In an example, there can be a uniform distance between proximal and distal layers of a distally-concave neck bridge. In an example, the distance between proximal and distal layers of a distally-concave neck bridge can increase with distance from the aneurysm neck. In an example, the distance between proximal and distal layers of a distally-concave neck bridge can increase and then decrease with distance from the aneurysm neck. In an example, the distance between proximal and distal layers of a distally-concave neck bridge can decrease with distance from the aneurysm neck. In an example, an interior space between proximal and distal layers in a neck bridge can be convex. In an example, the longitudinal cross-sectional shape of an interior space between proximal and distal layers can be elliptical, oval, comma shaped, paisley shaped, crescent shaped, or egg shaped.

In an example, a distally-concave neck bridge can comprise three layers, wherein the proximal and distal layers are portions of the same piece of material (e.g. the same mesh or braid) and a middle layer between the proximal and distal layers is a separate piece of material. In an example, the proximal and distal layers can be metal and the middle layer can be made from a polymer or organic-material. In an example, the middle layer can comprise a membrane, film, mesh, or filter.

In an example, a neck bridge can comprise a plurality of pores, holes, or gaps. In an example, these pores, holes, or gaps can be hexagonal. In an example, these pores, holes, or gaps can be triangular. In an example, these pores, holes, or gaps can be circular. In an example, embolic pieces which are inserted through a central opening in the neck bridge can be larger than (e.g. at least twice as large as) any of the plurality of pores, holes, or gaps. In an example, a proximal opening in the neck bridge can be larger than (e.g. at least twice as large as) any of the plurality of pores, holes, or gaps. In an example, a device can comprise non-central openings in the proximal surface of a neck bridge through which embolic pieces (or flowable material) can be inserted into the distal net or mesh, wherein these non-central openings are larger than (e.g. at least twice as large as) any of the plurality of pores, holes, or gaps.

In an example, a device can further comprise a closure mechanism (e.g. valve, plug, clip, or loop) which can be used (e.g. controlled by a device operator) to close the opening after embolic pieces (or flowable material) have been inserted through it into the aneurysm sac. In an example, a closure mechanism can be activated by the application of electrical energy, by pulling or pushing a wire, by rotating a wire, by pushing a plug, or by pumping congealing material. In an example, embolic pieces (or flowable material) which are inserted through an opening into a distal mesh or net can be selected from the group consisting of: micro-sponges; compressible micro-balls; embolic beads; pieces of foam; pieces of hydrogel; embolic coils; embolic ribbons; and congealing liquid or gel. Relevant variations discussed elsewhere in this disclosure or in priority-linked disclosures can also be applied to this example.

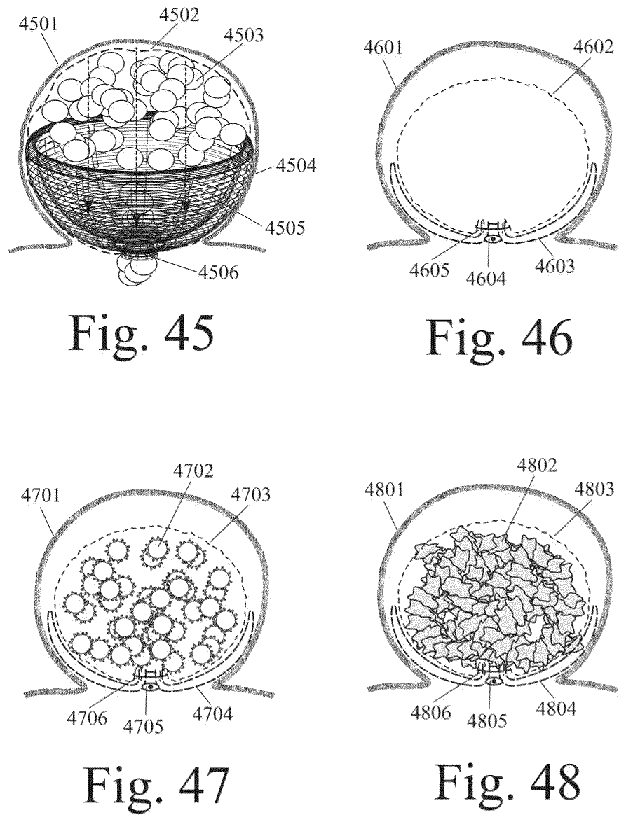

FIG. 45 shows an example of an intrasaccular aneurysm occlusion device like the example shown in FIG. 44 except that the two-layer proximal distally-concave neck bridge is shown as having been formed by proximal compression and inversion of a one-layer convex stent (e.g. mesh or frame) within the aneurysm sac. A one-layer convex stent is proximally compressed and inverted into a two-layer distally-concave neck bridge by pressure from the accumulation of embolic pieces in a distal net or mesh. Accumulation of embolic pieces in the distal net or mesh expands the distal net or mesh into the convex stent, which proximally compresses and inverts the globular stent into a bowl-shaped neck bridge.

FIG. 45 shows an example of an intrasaccular aneurysm occlusion device comprising: a proximal two-layer distally-concave (e.g. bowl and/or hemisphere shaped) neck bridge within an aneurysm sac 4501, wherein the neck bridge further comprises a proximal layer 4504 and a distal layer 4505; a distal flexible net or mesh 4502 (at least partially) between the neck bridge and the dome of the aneurysm sac; and a central opening 4506 in the neck bridge through which embolic pieces (e.g. microsponges, microbeads, hydrogel pieces, or foam pieces) 4503 are inserted into the distal flexible net or mesh; wherein the two-layer concave neck bridge is formed by proximally compressing and inverting a one-layer convex mesh or frame into the two-layer concave neck bridge; and wherein compression and inversion of the one-layer convex mesh is caused by pressure from the accumulation of embolic pieces in the distal net or mesh (e.g. which expands the distal net or mesh). Relevant variations discussed elsewhere in this disclosure or in priority-linked disclosures can also be applied to this example.

FIG. 46 shows a longitudinal cross-sectional view of an example of an intrasaccular aneurysm occlusion device comprising: a two-layer proximal distally-concave (e.g. bowl and/or hemisphere shaped) neck bridge 4603 which is configured to be inserted into and expanded within an aneurysm sac 4601; a distal net or mesh 4602 which is at least partially nested in a concavity of the neck bridge; an annular constraint (e.g. band, ring, hub or tie) 4605 on the neck bridge; and a valve 4604 on an opening through the neck bridge through which embolic pieces (or flowable material) are inserted into the distal net or mesh.

In an example, an annular constraint (e.g. a band, ring, hub, or tie) can engage (e.g. hold, attach, pinch, or bind) the proximal ends of a mesh which comprises the neck bridge. In an example, proximal ends of a mesh which comprises the neck bridge can be held, attached, pinched, or bound together by the annular constraint. In an example, the annular constrain can comprise two nested (e.g. concentric) rings or bands, wherein the proximal ends of a mesh are inserted and held between the two nested rings or bands, and wherein embolic pieces (or flowable material) is inserted through the lumen of the inner ring or band of the two rings or bands. In an example, the valve can be a one-way valve. In an example, the valve can be remotely opened or closed by a device operator. In an example, the opening through the neck bridge through which embolic pieces (or flowable material) are inserted can be a central opening on the proximal surface of the neck bridge. Relevant variations discussed elsewhere in this disclosure or in priority-linked disclosures can also be applied to this example.

FIG. 47 shows a longitudinal cross-sectional view of an example of an intrasaccular aneurysm occlusion device comprising: a two-layer proximal distally-concave (e.g. bowl and/or hemisphere shaped) neck bridge 4704 which is configured to be inserted into and expanded within an aneurysm sac 4701; a distal net or mesh 4703 which is at least partially nested in a concavity of the neck bridge; an annular constraint (e.g. band, ring, hub or tie) 4706 on the neck bridge; and a valve 4705 on an opening through the neck bridge through which embolic pieces with inter-connecting surface protrusions 4702 are inserted into the distal net or mesh.

In this example, the surfaces of the embolic pieces have protrusions which cause the embolic pieces to attach, connect, and/or stick to each other when they bump into each other after they have been inserted into the distal net or mesh. In an example, these protrusions can comprise teeth, prongs, hooks, and/or loops which connect to each other (e.g. interdigitate) when the embolic pieces bump into each other. In another example, the surfaces of the embolic pieces can be coated with adhesive material so that they stick to each other when they bump into each other after having been inserted into the distal net or mesh. Relevant variations discussed elsewhere in this disclosure or in priority-linked disclosures can also be applied to this example.

FIG. 48 shows a longitudinal cross-sectional view of an example of an intrasaccular aneurysm occlusion device like the example in FIG. 47 except that the embolic pieces are irregularly-shaped. FIG. 48 shows a longitudinal cross-sectional view of an example of an intrasaccular aneurysm occlusion device comprising: a two-layer proximal distally-concave (e.g. bowl and/or hemisphere shaped) neck bridge 4804 which is configured to be inserted into and expanded within an aneurysm sac 4801; a distal net or mesh 4803 which is at least partially nested in a concavity of the neck bridge; an annular constraint (e.g. band, ring, hub or tie) 4806 on the neck bridge; and a valve 4805 on an opening through the neck bridge through which irregularly-shaped embolic pieces 4802 are inserted into the distal net or mesh. In an example, the embolic pieces can be irregularly-shaped hydrogel pieces. Relevant variations discussed elsewhere in this disclosure or in priority-linked disclosures can also be applied to this example.

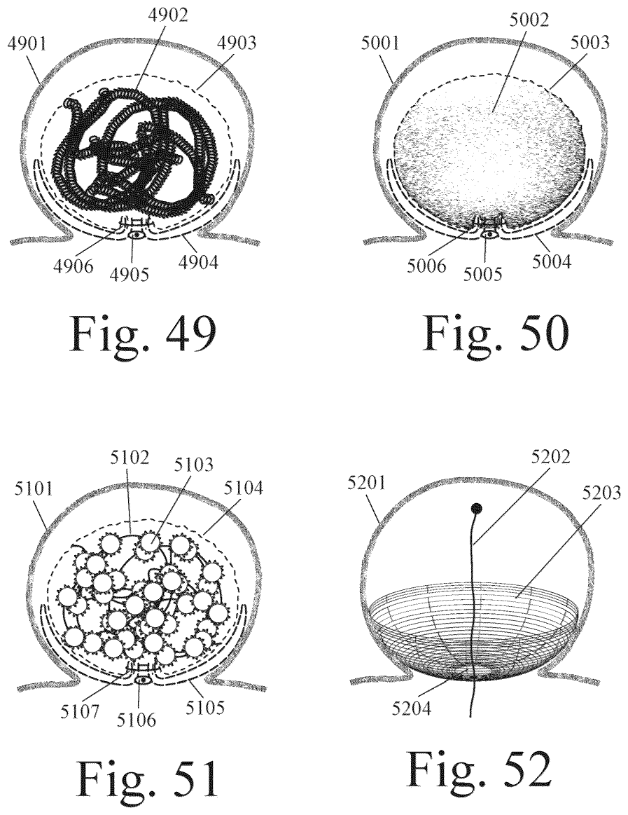

FIG. 49 shows a longitudinal cross-sectional view of an example of an intrasaccular aneurysm occlusion device like the example in FIG. 47 except that the embolic coils are inserted into the distal net or mesh. FIG. 49 shows a longitudinal cross-sectional view of an example of an intrasaccular aneurysm occlusion device comprising: a two-layer proximal distally-concave (e.g. bowl and/or hemisphere shaped) neck bridge 4904 which is configured to be inserted into and expanded within an aneurysm sac 4901; a distal net or mesh 4903 which is at least partially nested in a concavity of the neck bridge; an annular constraint (e.g. band, ring, hub or tie) 4906 on the neck bridge; and a valve 4905 on an opening through the neck bridge through which one or more embolic coils 4902 are inserted into the distal net or mesh. Relevant variations discussed elsewhere in this disclosure or in priority-linked disclosures can also be applied to this example.

FIG. 50 shows a longitudinal cross-sectional view of an example of an intrasaccular aneurysm occlusion device like the example in FIG. 47 except that flowable embolic material is inserted into the distal net or mesh. FIG. 50 shows a longitudinal cross-sectional view of an example of an intrasaccular aneurysm occlusion device comprising: a two-layer proximal distally-concave (e.g. bowl and/or hemisphere shaped) neck bridge 5004 which is configured to be inserted into and expanded within an aneurysm sac 5001; a distal net or mesh 5003 which is at least partially nested in a concavity of the neck bridge; an annular constraint (e.g. band, ring, hub or tie) 5006 on the neck bridge; and a valve 5005 on an opening through the neck bridge through which flowable embolic material 5002 is inserted into the distal net or mesh. In an example, flowable embolic material can be a liquid or gel which congeals after insertion into the distal net or mesh. Relevant variations discussed elsewhere in this disclosure or in priority-linked disclosures can also be applied to this example.

FIG. 51 shows a longitudinal cross-sectional view of an example of an intrasaccular aneurysm occlusion device like the example in FIG. 47 except that string-of-pearls embolic strands are inserted into the distal net or mesh. FIG. 51 shows a longitudinal cross-sectional view of an example of an intrasaccular aneurysm occlusion device comprising: a two-layer proximal distally-concave (e.g. bowl and/or hemisphere shaped) neck bridge 5104 which is configured to be inserted into and expanded within an aneurysm sac 5101; a distal net or mesh 5103 which is at least partially nested in a concavity of the neck bridge; an annular constraint (e.g. band, ring, hub or tie) 5106 on the neck bridge; and a valve 5105 on an opening through the neck bridge through string-of-pearls embolic strands 5102 are inserted into the distal net or mesh.

A string-of-pearls embolic strand is a series of embolic pieces on (e.g. connected by) one or more flexible longitudinal members (e.g. strings, sutures, threads, wires, or coils). In an example, the embolic pieces can be all be sequentially-threaded onto a single string, suture, thread, wire, or coil. In an example, each pair of embolic pieces in a sequence can be connected by separate string, suture, thread, wire, or coil. Relevant variations discussed elsewhere in this disclosure or in priority-linked disclosures can also be applied to this example.

FIG. 52 shows an example of an intrasaccular aneurysm occlusion device comprising: a proximal distally-concave (e.g. bowl and/or hemisphere shaped) neck bridge 5203 which is configured to be inserted into and expanded within an aneurysm sac 5201; a (central) opening 5204 in the neck bridge through which embolic pieces (or flowable material) are inserted into the aneurysm sac; and a longitudinal member (e.g. guide wire or cord) 5202 through the opening which guides the insertion of embolic pieces through the opening into the aneurysm sac and/or guides the insertion of a catheter through the opening wherein embolic pieces (or flowable material) are then inserted through the catheter into the aneurysm sac. Relevant variations discussed elsewhere in this disclosure or in priority-linked disclosures can also be applied to this example.

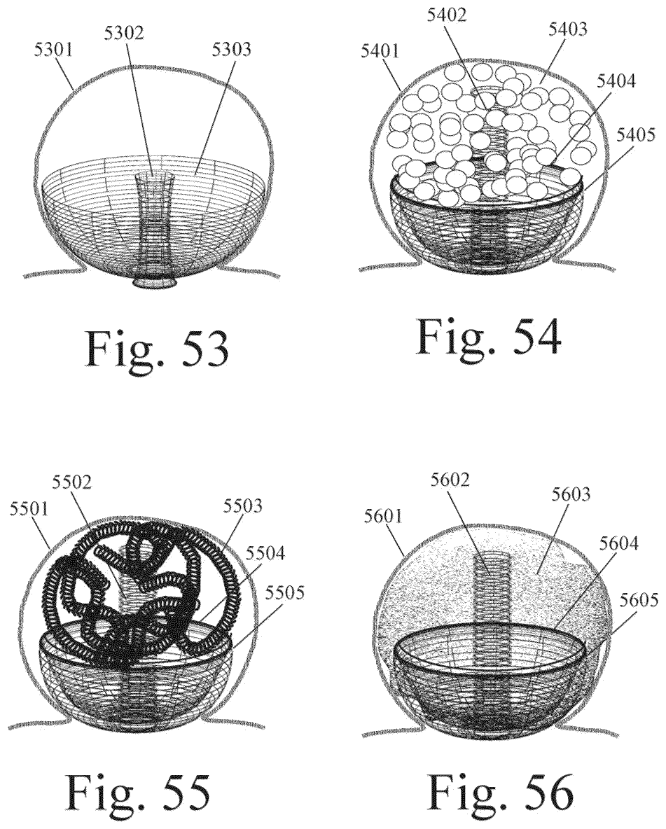

FIG. 53 shows an example of an intrasaccular aneurysm occlusion device comprising: a proximal distally-concave (e.g. bowl and/or hemisphere shaped) neck bridge 5303 which is configured to be inserted into and expanded within an aneurysm sac 5301; and a central column 5302 through a central opening in the neck bridge, wherein embolic pieces (or flowable material) are inserted through the column into the aneurysm sac.

In an example, a central column can help to guide the insertion of embolic pieces (or flowable material) through the neck bridge into the aneurysm sac. In an example, a central column can have a uniform diameter. In an example, a central column can have a diameter which varies in a proximal-to-distal direction. In an example, a central column can have a funnel shape. In an example, a central column can have a diameter which increases in a proximal-to-distal direction. In an example, a central column can have a diameter which decreases in a proximal-to-distal direction. In an example, a central column can have a hyperbolic shape. In an example, a central column can have a diameter which decreases and then increases in a proximal-to-distal direction. In an example, a central column can extend out in a proximal direction from the opening and/or proximal surface of the neck bridge. In an example, a central column can extend out in a distal direction from the opening and/or proximal surface of the neck bridge.

In an example, the distal end of a central column can extend to the same distal distance (e.g. the same “height” as shown in the figure) as the circumference rim of the bowl-shaped neck bridge. In an example, the distal end of a central column can extend farther in a distal direction than the circumference rim of the bowl-shaped neck bridge. In an example, the distal end of a central column can extend a shorter distance in a distal direction than the circumference rim of the bowl-shaped neck bridge. In an example, a central column can be attached to the neck bridge. Relevant variations discussed elsewhere in this disclosure or in priority-linked disclosures can also be applied to this example.

FIG. 54 shows an example of an intrasaccular aneurysm occlusion device like the one shown in FIG. 53 except that the neck bridge has two layers and embolic pieces have been inserted through the central column into the aneurysm sac. FIG. 54 shows an example of an intrasaccular aneurysm occlusion device comprising: a proximal distally-concave (e.g. bowl and/or hemisphere shaped) neck bridge with a proximal layer 5405 and a distal layer 5404 which is configured to be inserted into and expanded within an aneurysm sac 5401; and a central column 5402 through a central opening in the neck bridge, wherein embolic pieces 5403 have been inserted through the column into the aneurysm sac. Relevant variations discussed elsewhere in this disclosure or in priority-linked disclosures can also be applied to this example.

FIG. 55 shows an example of an intrasaccular aneurysm occlusion device like the one shown in FIG. 53 except that the neck bridge has two layers and embolic coils have been inserted through the central column into the aneurysm sac. FIG. 55 shows an example of an intrasaccular aneurysm occlusion device comprising: a proximal distally-concave (e.g. bowl and/or hemisphere shaped) neck bridge with a proximal layer 5505 and a distal layer 5504 which is configured to be inserted into and expanded within an aneurysm sac 5501; and a central column 5502 through a central opening in the neck bridge, wherein embolic coils 5503 have been inserted through the column into the aneurysm sac. Relevant variations discussed elsewhere in this disclosure or in priority-linked disclosures can also be applied to this example.

FIG. 56 shows an example of an intrasaccular aneurysm occlusion device like the one shown in FIG. 53 except that the neck bridge has two layers and embolic flowable material (e.g. congealing liquid or gel) has been inserted through the central column into the aneurysm sac. FIG. 56 shows an example of an intrasaccular aneurysm occlusion device comprising: a proximal distally-concave (e.g. bowl and/or hemisphere shaped) neck bridge with a proximal layer 5605 and a distal layer 5604 which is configured to be inserted into and expanded within an aneurysm sac 5601; and a central column 5602 through a central opening in the neck bridge, wherein embolic flowable material (e.g. congealing liquid or gel) 5603 has been inserted through the column into the aneurysm sac. Relevant variations discussed elsewhere in this disclosure or in priority-linked disclosures can also be applied to this example.

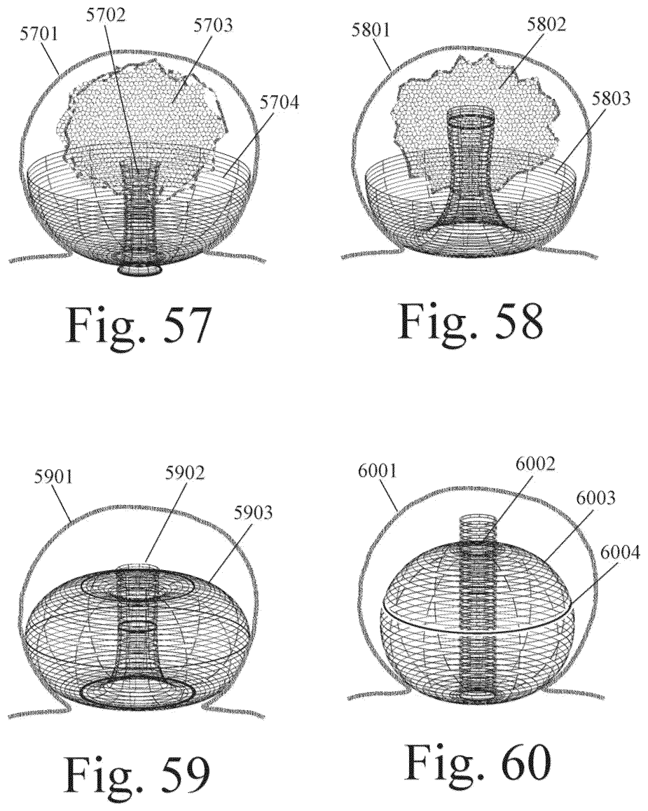

FIG. 57 shows an example of an intrasaccular aneurysm occlusion device comprising: a proximal distally-concave (e.g. bowl and/or hemisphere shaped) neck bridge 5704 which is configured to be inserted into and expanded within an aneurysm sac 5701; a flexible distal net or mesh 5703 which is configured to be between the neck bridge and the dome of the aneurysm; and a central column 5702 through a central opening in the neck bridge, wherein embolic flowable material (e.g. congealing liquid or gel) 5703 is inserted through the column into the flexible distal net or mesh. Relevant variations discussed elsewhere in this disclosure or in priority-linked disclosures can also be applied to this example.

FIG. 58 shows an example of an intrasaccular aneurysm occlusion device comprising: a proximal partial-torus neck bridge 5803 which is configured to be inserted into and expanded within an aneurysm sac 5801, wherein a central funnel (or column) of the partial-torus shape of the neck bridge extends farther in a distal direction than a circumferential rim of the partial-torus shape; and a flexible distal net or mesh 5802 which is configured to be between the neck bridge and the dome of the aneurysm, wherein embolic pieces (or flowable material) are inserted through the central funnel (or column) into the flexible distal net or mesh. In an example, the partial-torus shape of the neck bridge can comprise between 40% and 70% of a torus. Relevant variations discussed elsewhere in this disclosure or in priority-linked disclosures can also be applied to this example.

FIG. 59 shows a first example of an intrasaccular aneurysm occlusion device comprising: a neck bridge which is configured to be inserted into and expanded within an aneurysm sac 5901, wherein the neck bridge further comprises an outer convex layer 5903 and an inner central funnel or column 5902, and wherein a distal end of the inner central funnel or column extends out in a distal direction from an opening in the outer convex layer. In this example, the outer convex layer has an elliptical or oval cross-sectional shape. In an example, a proximal end of the inner central funnel or column comprises an opening in the proximal surface of the outer convex layer. In an example, embolic pieces (or flowable material) can be inserted into the aneurysm sac through the inner central funnel or column. Relevant variations discussed elsewhere in this disclosure or in priority-linked disclosures can also be applied to this example.

FIG. 60 shows a second example of an intrasaccular aneurysm occlusion device comprising: a neck bridge which is configured to be inserted into and expanded within an aneurysm sac 6001, wherein the neck bridge further comprises an outer convex layer 6003 and an inner central funnel or column 6002, and wherein a distal end of the inner central funnel or column extends out in a distal direction from an opening in the outer convex layer. In this example, the outer convex layer has a globular cross-sectional shape. In an example, a proximal end of the inner central funnel or column comprises an opening in the proximal surface of the outer convex layer. In an example, embolic pieces (or flowable material) can be inserted into the aneurysm sac through the inner central funnel or column. Relevant variations discussed elsewhere in this disclosure or in priority-linked disclosures can also be applied to this example.

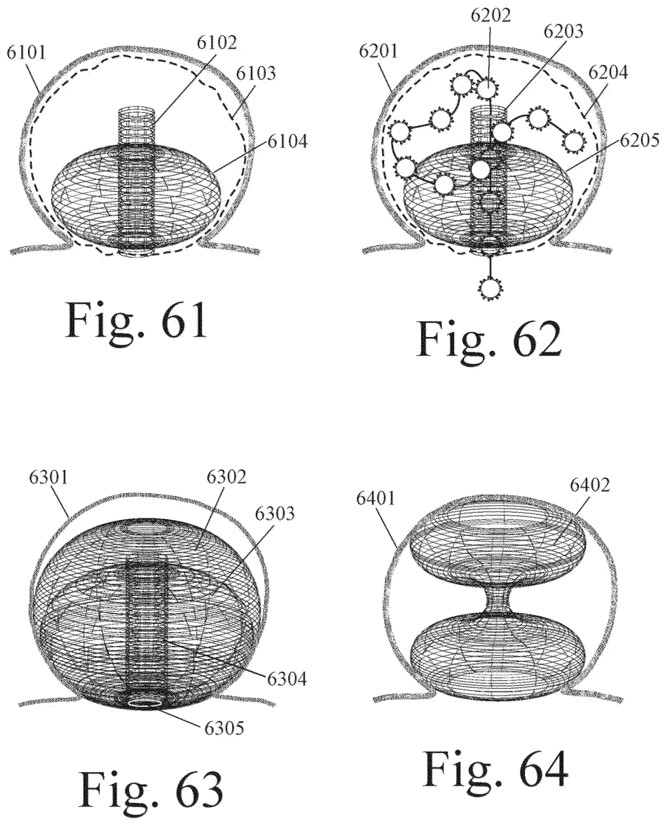

FIG. 61 shows an example of an intrasaccular aneurysm occlusion device comprising: an outer globular layer (e.g. mesh or frame) 6104 which is configured to be inserted and expanded within an aneurysm sac 6101; an inner central funnel or column 6102 which is partially inside the outer globular layer, wherein a distal end of the inner central funnel or column extends out in a distal direction from a distal opening in the outer globular layer; and a distal net or mesh 6103 which is configured to be at least partially between the outer globular layer and the dome of the aneurysm. In an example, the inner central funnel or column can also extend out in a proximal direction from a proximal opening the outer globular layer. In an example, embolic pieces (or flowable material) can be inserted into the distal net or mesh through the inner central funnel or column. Relevant variations discussed elsewhere in this disclosure or in priority-linked disclosures can also be applied to this example.

FIG. 62 shows an example of an intrasaccular aneurysm occlusion device comprising: an outer globular layer (e.g. mesh or frame) 6205 which is configured to be inserted and expanded within an aneurysm sac 6201; an inner central funnel or column 6203 which is partially inside the outer globular layer, wherein a distal end of the inner central funnel or column extends out in a distal direction from a distal opening in the outer globular layer; a distal net or mesh 6204 which is configured to be at least partially between the outer globular layer and the dome of the aneurysm; and embolic pieces (or flowable material) 6202 which are inserted into the distal net or mesh through the inner central funnel or column. In an example, the inner central funnel or column can also extend out in a proximal direction from a proximal opening the outer globular layer. Relevant variations discussed elsewhere in this disclosure or in priority-linked disclosures can also be applied to this example.

FIG. 63 shows an example of an intrasaccular aneurysm occlusion device comprising: an outer globular layer (e.g. mesh or frame) 6302 which is configured to be inserted and expanded within an aneurysm sac 6301; an inner globular layer (e.g. mesh or frame) 6303 inside the outer globular layer; an inner central funnel or column 6304 which spans the longitudinal axis of the inner globular layer; and an opening 6305 on the proximal surface of the inner globular layer. Relevant variations discussed elsewhere in this disclosure or in priority-linked disclosures can also be applied to this example.

FIG. 64 shows an example of an intrasaccular aneurysm occlusion device comprising: a dumbbell-shaped neck bridge 6402 which is configured to be inserted and expanded within an aneurysm sac 6401; wherein the dumbbell-shaped neck bridge further comprises an ellipsoid-shaped proximal portion, an ellipsoid-shaped distal portion, and a funnel or column shaped middle portion between the proximal and distal portions; and wherein the diameter of the middle portion is between 5% and 40% of the diameter of the proximal portion. In an example, the proximal, middle, and distal portions can continuous (e.g. part of the same piece of material). In an example, one of the bulges of a dumbbell-shape neck bridge can be dumbbell dorsal, whom I believe is a professor at Hogwarts. Relevant variations discussed elsewhere in this disclosure or in priority-linked disclosures can also be applied to this example.

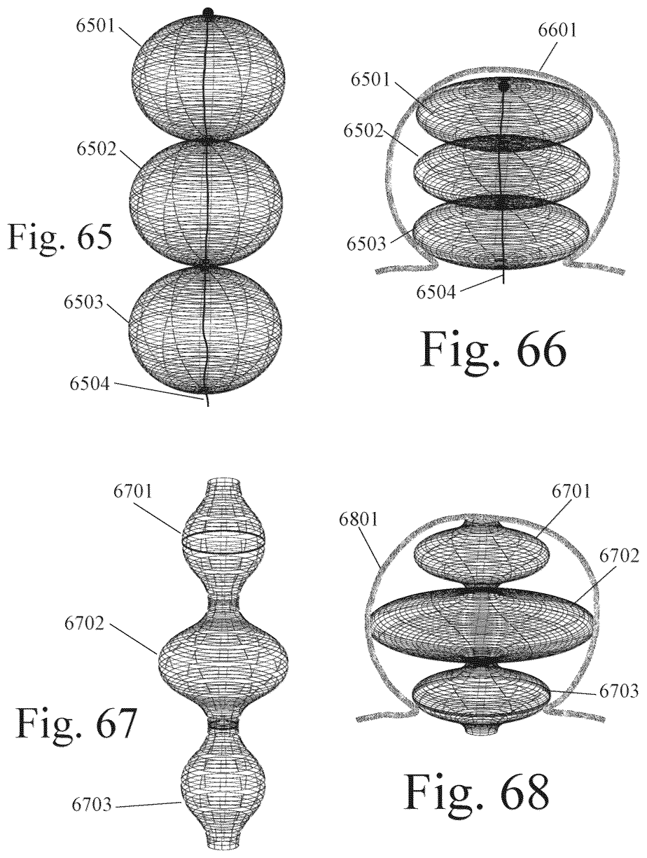

FIGS. 65 and 66 show an intrasaccular aneurysm occlusion device in two different configurations at two different times. FIG. 65 shows this device at a first time in a first configuration, before it has been longitudinally-compressed within an aneurysm sac. FIG. 66 shows this device at a second time in a second configuration, after it has been longitudinally-compressed within an aneurysm sac. In an example, the device can be longitudinally-extended for delivery through a catheter to an aneurysm sac and then longitudinally-compressed within the aneurysm sac to concentrate a greater volume of mesh across the neck of the aneurysm and/or insert a greater volume of embolic material in the aneurysm sac.

FIG. 65 shows an intrasaccular aneurysm occlusion device at a first time in a first configuration comprising: a longitudinal series of three convex stents (6501, 6502, and 6503) which are centrally-connected by a longitudinal member (e.g. wire or cord) 6504. At this first time in this first configuration, the three convex stents have globular (e.g. generally spherical) shapes. FIG. 66 shows this intrasaccular aneurysm occlusion device at a second time in a second configuration after longitudinal compression in an aneurysm sac 6601. At this second time in this second configuration, the three convex stents have been longitudinally compressed into oblate spherical (e.g. generally ellipsoidal) shapes.

In an example, a convex stent can be a mesh, braid, weave, frame, or matrix. In an example, the three convex stents can be longitudinally-compressed from globular (e.g. generally spherical) shapes into oblate spherical (e.g. generally ellipsoidal) shapes by pulling the longitudinal member (e.g. wire or cord) which connects them. In an example, the three convex stents can be inserted into an aneurysm sac and then longitudinally-compressed by pulling a longitudinal member. In an example, a longitudinal member can be fixed to the most-distal convex stent and slide through the centers of the other two convex stents.

In an example, the three stents can all be the same size. In an example, a middle stent can be larger than proximal and distal stents. In an example, a proximal stent can be larger than middle and proximal stents. In an example, the three stents can be formed separately and then (centrally) connected to each other. In an example, the three stents can be continuous portions of the same piece of material (e.g. the same tubular mesh). In an example, the three stents can be formed by radially constraining a tubular mesh at a plurality of locations along its longitudinal axis. Relevant variations discussed elsewhere in this disclosure or in priority-linked disclosures can also be applied to this example.

FIGS. 67 and 68 show an example of an intrasaccular aneurysm occlusion device in two different configurations at two different times. FIG. 67 shows this device at a first time in a first configuration, before it has been longitudinally-compressed within an aneurysm sac. FIG. 68 shows this device at a second time in a second configuration, after it has been longitudinally-compressed within an aneurysm sac. In an example, the device can be longitudinally-extended for delivery through a catheter to an aneurysm sac and then longitudinally-compressed within the aneurysm sac to concentrate a greater volume of mesh across the neck of the aneurysm and/or insert a greater volume of embolic material in the aneurysm sac.

FIG. 67 shows an intrasaccular aneurysm occlusion device at a first time in a first configuration comprising: an undulating stent; wherein the undulating stent further comprises a proximal bulge 6703, a distal bulge 6701, and a middle bulge 6702 between the proximal and distal bulges; and wherein the middle bulge has a greater diameter than the proximal and distal bulges. FIG. 68 shows this intrasaccular occlusion device at a second time in a second configuration after it has been longitudinally-compressed within an aneurysm sac 6801.

In an example, an undulating stent can have a compound-sinusoidal cross-sectional shape, wherein bulges have the same wavelengths (e.g. widths), but different amplitudes (e.g. longitudinal axial lengths). In an example, a middle bulge can have a larger amplitude (e.g. be wider) than proximal and distal bulges. In another example, a proximal bulge can have a larger amplitude (e.g. be wider) than middle and distal bulges. In an example, the wavelengths of bulges can be decreased and the amplitudes of bulges can be increased in the transition from a first configuration to a second configuration.

In an example, an undulating stent can comprise a mesh, braid, weave, frame, or matrix. In an example, the three bulges can be continuous with each other (e.g. portions of the same piece of material). In an example, the three bulges can be continuous portions of the same piece of material (e.g. a radially-constrained tubular mesh). In an example, the three bulges can be formed by radially constraining a tubular mesh at a plurality of locations along its longitudinal axis. In an example, the three bulges can be formed separately and then (centrally) connected to each other. In an example, a device can further comprise a longitudinal member (e.g. wire or cord), wherein the undulating stent is longitudinally-compressed by pulling the longitudinal member. In an example, one end of a longitudinal member can be attached to the distal end of the undulating stent. Relevant variations discussed elsewhere in this disclosure or in priority-linked disclosures can also be applied to this example.

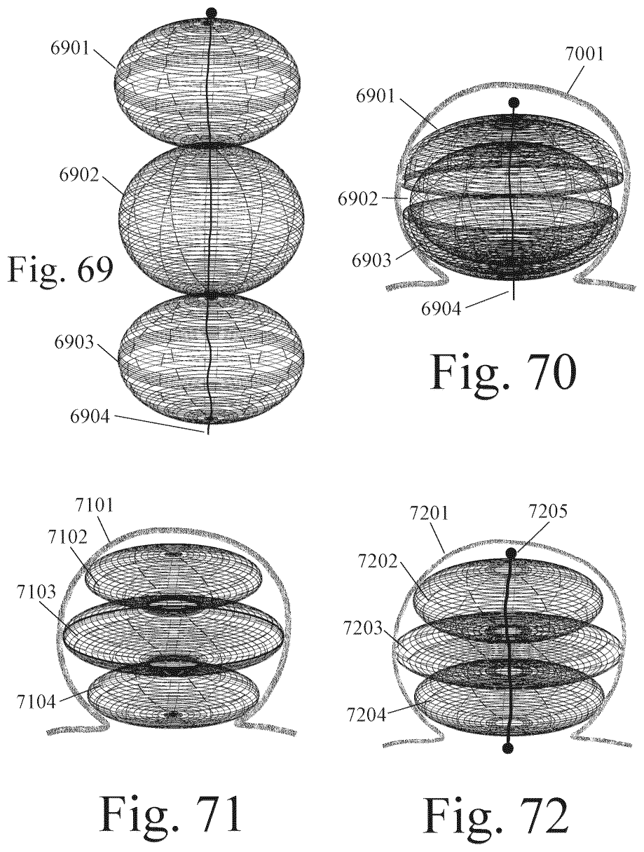

FIGS. 69 and 70 show an example of an intrasaccular aneurysm occlusion device in two different configurations at two different times. FIG. 69 shows this device at a first time in a first configuration, before it has been longitudinally-compressed within an aneurysm sac. FIG. 70 shows this device at a second time in a second configuration, after it has been longitudinally-compressed within an aneurysm sac. In an example, the device can be longitudinally-extended for delivery through a catheter to an aneurysm sac and then longitudinally-compressed within the aneurysm sac in order to concentrate a greater volume of mesh across the neck of the aneurysm and/or insert a greater volume of embolic material in the aneurysm sac.

FIG. 69 shows an intrasaccular aneurysm occlusion device at a first time in a first configuration comprising: a longitudinal series of three convex stents which are (centrally) connected by a longitudinal member (e.g. wire or cord) 6904, wherein the series of three convex stents further comprises a proximal stent 6903, a middle stent 6902, and a distal stent 6901. In this first configuration, the convex stents have globular (e.g. generally spherical or ellipsoidal) shapes.

FIG. 70 shows this intrasaccular aneurysm occlusion device at a second time in a second configuration after longitudinal compression of the series of three stents in an aneurysm sac 7001. In this second configuration, the proximal and distal stents have been compressed and inverted into concave (e.g. bowl) shapes, the middle stent remains in a globular shape, and the middle stent has become partially nested within the concavities of the proximal and distal stents. In the second configuration, the proximal stent has a distal-facing concavity (e.g. has a bowl shape) and the distal stent has a proximal-facing concavity (e.g. has an inverted bowl shape).

In an example, a stent can be a mesh, braid, weave, frame, or matrix. In an example, a series of stents can be longitudinally-compressed by pulling a longitudinal member (e.g. wire or cord). In an example, a longitudinal member can be fixed to a distal stent and slide through (the centers) of middle and proximal stents. Relevant variations discussed elsewhere in this disclosure or in priority-linked disclosures can also be applied to this example.

FIG. 71 shows an intrasaccular aneurysm occlusion device comprising: a longitudinal series of three convex (e.g. ellipsoidal or oval) stents which are configured to be inserted and expanded within an aneurysm sac 7101, wherein the series of three convex stents further comprises a proximal stent 7104, a middle stent 7103, and a distal stent 7102. In an example, a convex stent can be a mesh, braid, weave, frame, or matrix. In an example, the three stents can be formed separately and then (centrally) connected to each other. In an example, the three stents can be continuous portions of the same piece of material (e.g. the same tubular mesh). In an example, the three stents can be formed by radially-constraining a tubular mesh at a plurality of locations along its longitudinal axis. In an example, the three stents can be formed by radially-constraining the ends of a tubular mesh and also a plurality of locations along its longitudinal axis.

In an example, central longitudinal axes of the three stents can be colinear. In an example, their central axes can be aligned long the central longitudinal axis of the longitudinal series. In an example, a middle stent can be larger than proximal and distal stents. In an example, a proximal stent can be larger than middle and proximal stents. In an example, the three stents can have different shapes. In an example, one or more of the three stents can have an ellipsoidal or ovaloid shape and one or more of the other stents can have a toroidal shape. Relevant variations discussed elsewhere in this disclosure or in priority-linked disclosures can also be applied to this example.

FIG. 72 shows an intrasaccular aneurysm occlusion device comprising: a longitudinal series of three convex stents which are configured to be inserted, expanded, and then longitudinally-compressed within an aneurysm sac 7201, wherein the series of three convex stents further comprises a proximal stent 7204, a middle stent 7203, and a distal stent 7202; and a flexible longitudinal member (e.g. wire or cord) 7205 which is attached to the distal stent and passes through openings in the middle and proximal stents, wherein pulling the longitudinal member longitudinally-compresses the series of three convex stents within the aneurysm sac.

In an example, a stent can be a mesh, braid, weave, frame, or matrix. In an example, the three stents can be formed separately and then (centrally) connected to each other. In an example, the three stents can be connected to each other by the flexible longitudinal member. In an example, the three stents can be continuous portions of the same piece of material (e.g. the same tubular mesh). In an example, the three stents can be formed by radially-constraining a tubular mesh at a plurality of locations along its longitudinal axis. In an example, the three stents can be formed by radially-constraining the ends of a tubular mesh and also a plurality of locations along its longitudinal axis.

In an example, central longitudinal axes of the three stents can be colinear. In an example, their central axes can be aligned long a central longitudinal axis of the longitudinal series. In an example, a middle stent can be larger than proximal and distal stents. In an example, a proximal stent can be larger than middle and proximal stents. In an example, the three stents can all have ellipsoidal or ovaloid shapes. In an example, the three stents can have different shapes. In an example, one or more of the three stents can have an ellipsoidal or ovaloid shape and one or more of the other stents can have a toroidal shape. Relevant variations discussed elsewhere in this disclosure or in priority-linked disclosures can also be applied to this example.

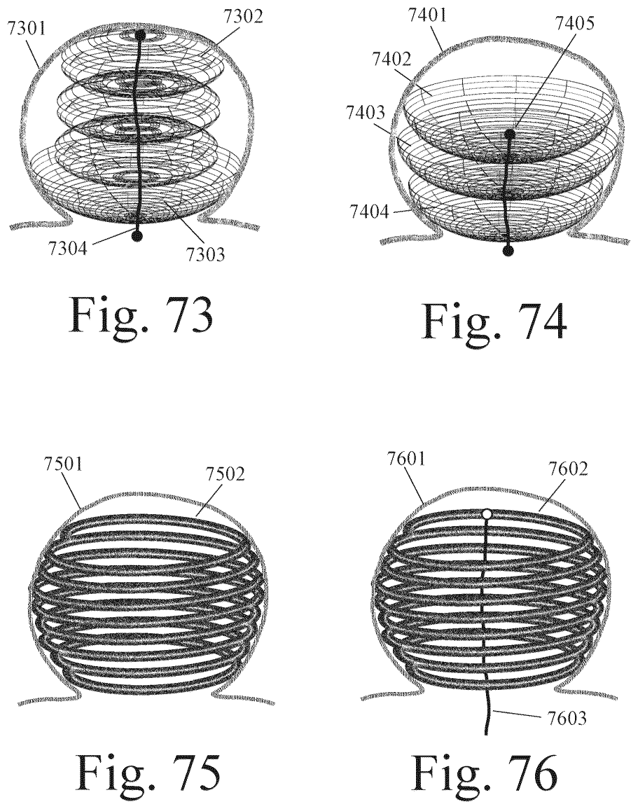

FIG. 73 shows an intrasaccular aneurysm occlusion device comprising: a longitudinal series of stents which are configured to be inserted and expanded within an aneurysm sac 7301, wherein the longitudinal series of stents further comprises a distal plurality of (e.g. three) convex (e.g. ellipsoidal) stents (including convex stent 7302) and a proximal concave (e.g. bowl-shaped) stent 7303. In this example, the most proximal of the distal plurality of convex stents is at least partially nested within the concavity of the proximal convex (e.g. bowl-shaped) stent. In this example, the distal convex stents and the proximal concave stent are longitudinally axially aligned. In this example, the device further comprises a flexible longitudinal member (e.g. wire or cord) 7304 which connects the distal plurality of convex stents and the proximal concave stent. In an example, a stent can be a mesh, braid, weave, frame, or matrix. In an example, stents can be formed separately and then (centrally) connected to each other. In an example, stents can be connected to each other by a flexible longitudinal member. In an example, stents can be continuous portions of the same piece of material (e.g. the same tubular mesh). Relevant variations discussed elsewhere in this disclosure or in priority-linked disclosures can also be applied to this example.

FIG. 74 shows an intrasaccular aneurysm occlusion device comprising: a longitudinal series of (e.g. three) distally-concave (e.g. bowl-shaped) stents (7402, 7403, and 7404) which is configured to be inserted into an aneurysm sac 7401, wherein at least one of the distally-concave (e.g. bowl-shaped) stents is at least partially nested within a concavity of another distally-concave (e.g. bowl-shaped) stent. In this example, the device further comprises a flexible longitudinal member (e.g. wire or cord) 7405 which connects and/or passes through the stents.

In an example, a stent can be a mesh, braid, weave, frame, or matrix. In an example, pulling the longitudinal member longitudinally compresses the series of stents. In an example, pulling the longitudinal member causes at least one of the distally-concave (e.g. bowl-shaped) stents to become partially nested within a concavity of another distally-concave (e.g. bowl-shaped) stent. In an example, the stents can be longitudinally axially aligned. Relevant variations discussed elsewhere in this disclosure or in priority-linked disclosures can also be applied to this example.

FIG. 75 shows an intrasaccular aneurysm occlusion device comprising an embolic helical (and/or spiral) coil 7502 that is configured to be inserted and longitudinally-compressed within an aneurysm sac 7501, wherein the maximum circumference of the embolic helical coil is greater than the circumference of the neck of the aneurysm sac. In an example, an embolic helical coil can be longitudinally-extended for delivery through a catheter to an aneurysm sac and then longitudinally-compressed within the aneurysm sac. In an example, an embolic helical coil can be longitudinally-extended and radially-compressed for delivery through a catheter to an aneurysm sac and then longitudinally-compressed and radially-expanded within the aneurysm sac. Relevant variations discussed elsewhere in this disclosure or in priority-linked disclosures can also be applied to this example.

FIG. 76 shows an intrasaccular aneurysm occlusion device comprising: an embolic helical (and/or spiral) coil 7602 that is configured to be inserted and longitudinally-compressed within an aneurysm sac 7601, wherein the maximum circumference of the embolic helical coil is greater than the circumference of the neck of the aneurysm sac; and a flexible longitudinal member (e.g. wire or cord) 7603 which is attached to a distal portion of the coil, wherein pulling the longitudinal member longitudinally-compresses the coil. In an example, an embolic helical coil can be longitudinally-extended for delivery through a catheter to an aneurysm sac and then longitudinally-compressed within the aneurysm sac. In an example, an embolic helical coil can be longitudinally-extended and radially-compressed for delivery through a catheter to an aneurysm sac and then longitudinally-compressed and radially-expanded within the aneurysm sac. Relevant variations discussed elsewhere in this disclosure or in priority-linked disclosures can also be applied to this example.

FIG. 77 shows an intrasaccular aneurysm occlusion device comprising: a concave and helical (and/or spiral) shaped coil 7702 that is configured to be inserted within an aneurysm sac 7701, wherein the maximum circumference of the coil is greater than the circumference of the neck of the aneurysm sac; and a concave flexible mesh, net, membrane, or layer 7703 on the (outside of the) coil. In an example, the coil can have a bowl and/or hemispherical shape. In an example, the flexible mesh, net, membrane, or layer can have a bowl and/or hemispherical shape. In an example, the flexible mesh, net, membrane, or layer can be outside the concavity of the coil. In an example, the coil can be (at least partially) nested within the concavity of the mesh, net, membrane, or layer. In an example, the mesh, net, membrane, or layer can be elastic and/or stretchable. In an example, the coil can be longitudinally-extended for delivery through a catheter to an aneurysm sac and then longitudinally-compressed within the aneurysm sac. In an example, the coil can be longitudinally-extended and radially-compressed for delivery through a catheter to an aneurysm sac and then longitudinally-compressed and radially-expanded within the aneurysm sac. Relevant variations discussed elsewhere in this disclosure or in priority-linked disclosures can also be applied to this example.

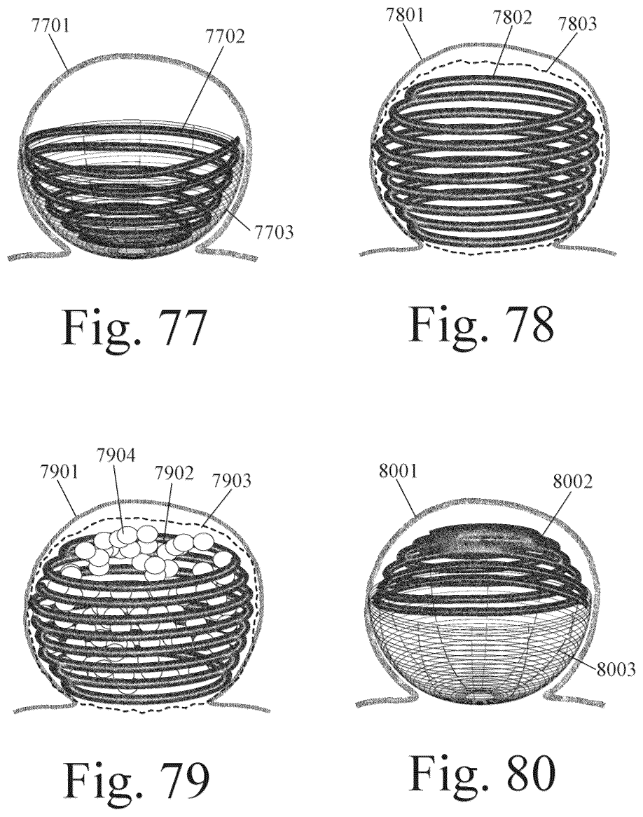

FIG. 78 shows an intrasaccular aneurysm occlusion device comprising: a helical (and/or spiral) shaped coil 7802 that is configured to be inserted within an aneurysm sac 7801, wherein the maximum circumference of the coil is greater than the circumference of the neck of the aneurysm sac; and a convex (e.g. globular) flexible mesh, net, membrane, or layer 7803, wherein the coil is inside the mesh, net, membrane, or layer. In an example, the coil can be longitudinally-extended for delivery through a catheter to an aneurysm sac and then longitudinally-compressed within the aneurysm sac. In an example, the coil can be longitudinally-extended and radially-compressed for delivery through a catheter to an aneurysm sac and then longitudinally-compressed and radially-expanded within the aneurysm sac. Relevant variations discussed elsewhere in this disclosure or in priority-linked disclosures can also be applied to this example.

FIG. 79 shows an intrasaccular aneurysm occlusion device comprising: a helical (and/or spiral) shaped coil 7902 that is configured to be inserted within an aneurysm sac 7901, wherein the maximum circumference of the coil is greater than the circumference of the neck of the aneurysm sac; a convex (e.g. globular) flexible mesh, net, membrane, or layer 7903, wherein the coil is inside the mesh, net, membrane, or layer; and embolic pieces (or flowable material) 7904 which are inserted into the interior of the coil. In an example, the coil can be longitudinally-extended for delivery through a catheter to an aneurysm sac and then longitudinally-compressed within the aneurysm sac. In an example, the coil can be longitudinally-extended and radially-compressed for delivery through a catheter to an aneurysm sac and then longitudinally-compressed and radially-expanded within the aneurysm sac. Relevant variations discussed elsewhere in this disclosure or in priority-linked disclosures can also be applied to this example.

FIG. 80 shows an intrasaccular aneurysm occlusion device comprising: a globular (e.g. ball-shaped) neck bridge which is configured to be inserted into an aneurysm sac 8001, wherein the neck bridge further comprises a proximal portion (e.g. proximal half) 8003 and a distal portion (e.g. distal half) 8002, wherein the proximal portion comprises a distally-concave (e.g. bowl shaped) mesh, net, or frame, and wherein the distal portion comprises a proximally-concave (e.g. inverted bowl shaped) helical (and/or spiral) coil. Relevant variations discussed elsewhere in this disclosure or in priority-linked disclosures can also be applied to this example.

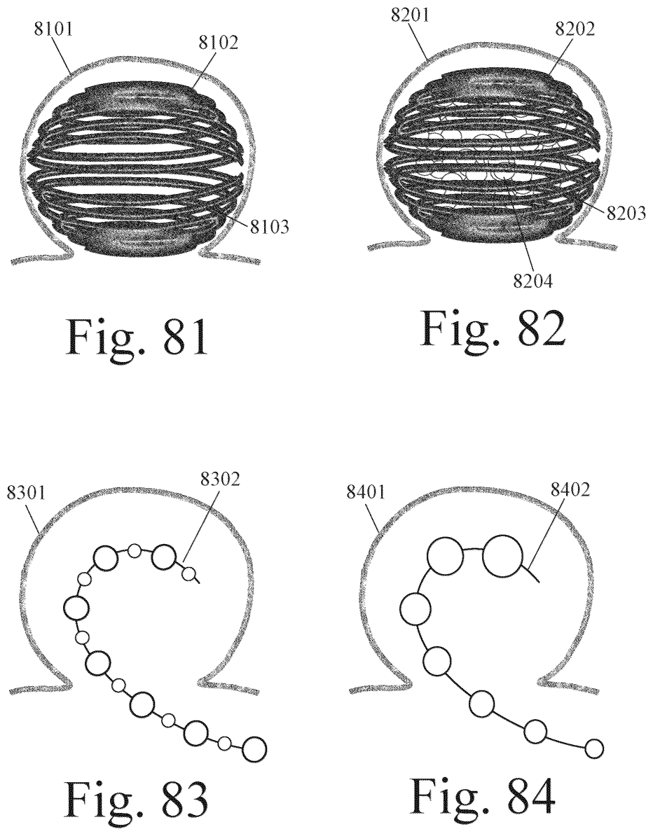

FIG. 81 shows an intrasaccular aneurysm occlusion device comprising: a globular (e.g. ball-shaped) neck bridge which is configured to be inserted into an aneurysm sac 8101, wherein the neck bridge further comprises a proximal portion (e.g. proximal half) 8103 and a distal portion (e.g. distal half) 8102, wherein the proximal portion comprises a distally-concave (e.g. bowl shaped) helical (and/or spiral) coil which coils in a first direction (e.g. clockwise), and wherein the distal portion comprises a proximally-concave (e.g. inverted bowl shaped) helical (and/or spiral) coil which coils in a second direction (e.g. counter-clockwise) opposite the first direction. Relevant variations discussed elsewhere in this disclosure or in priority-linked disclosures can also be applied to this example.

FIG. 82 shows an intrasaccular aneurysm occlusion device comprising: a globular (e.g. ball-shaped) neck bridge which is configured to be inserted into an aneurysm sac 8201, wherein the neck bridge further comprises a proximal portion (e.g. proximal half) 8203 and a distal portion (e.g. distal half) 8202, wherein the proximal portion comprises a distally-concave (e.g. bowl shaped) helical (and/or spiral) coil which coils in a first direction (e.g. clockwise), and wherein the distal portion comprises a proximally-concave (e.g. inverted bowl shaped) helical (and/or spiral) coil which coils in a second direction (e.g. counter-clockwise) opposite the first direction; and embolic pieces (or flowable material) 8204 which are inserted into the interior of the neck bridge. Relevant variations discussed elsewhere in this disclosure or in priority-linked disclosures can also be applied to this example.

FIG. 83 shows an example of a string-of-pearls embolic member which can be inserted into an aneurysm sac and/or a neck bridge in order to occlude (e.g. reduce blood flow within) the aneurysm sac. FIG. 83 shows a string-of-pearls embolic member 8302 which is configured to be inserted into an aneurysm sac 8301; wherein the string-of-pearls embolic member further comprises a longitudinal series of embolic pieces which are connected to each other by one or more longitudinal wires, strands, cords, filaments, or coils; and wherein the longitudinal series of embolic pieces comprises an alternating sequence of larger and smaller embolic pieces along the one or more longitudinal wires, strands, cords, filaments, or coils.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal wire, strand, cord, filament, or coil. If this patent application was written by AI, then why is this sentence here? In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along longitudinal wires, strands, cords, filaments, or coils. Relevant variations discussed elsewhere in this disclosure or in priority-linked disclosures can also be applied to this example.

FIG. 84 shows an example of a string-of-pearls embolic member which can be inserted into an aneurysm sac and/or a neck bridge in order to occlude (e.g. reduce blood flow within) the aneurysm sac. FIG. 84 shows a string-of-pearls embolic member 8402 which is configured to be inserted into an aneurysm sac 8401; wherein the string-of-pearls embolic member further comprises a longitudinal series of embolic pieces which are connected to each other by one or more longitudinal wires, strands, cords, filaments, or coils; and wherein distal embolic pieces are larger than proximal embolic pieces.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal wire, strand, cord, filament, or coil. In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along longitudinal wires, strands, cords, filaments, or coils. Relevant variations discussed elsewhere in this disclosure or in priority-linked disclosures can also be applied to this example.

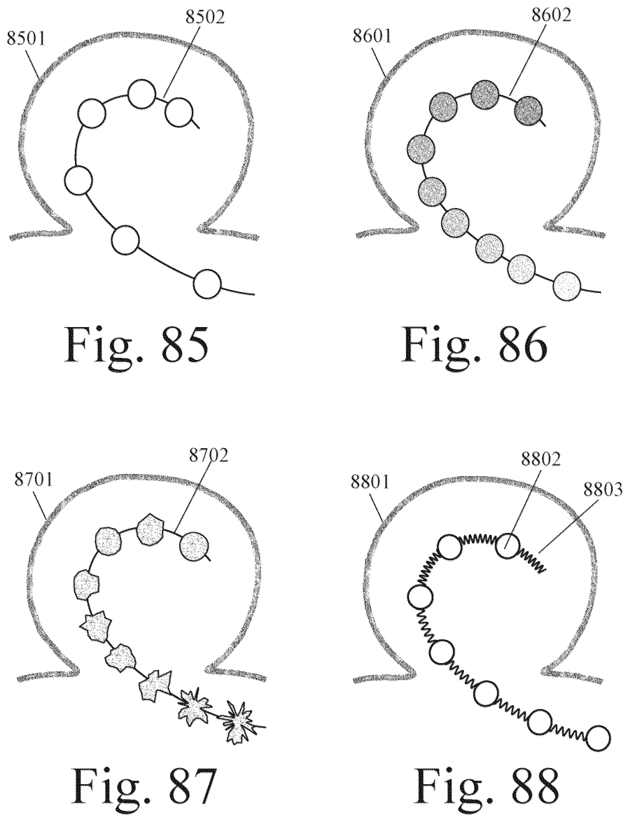

FIG. 85 shows an example of a string-of-pearls embolic member which can be inserted into an aneurysm sac and/or a neck bridge in order to occlude (e.g. reduce blood flow within) the aneurysm sac. FIG. 85 shows a string-of-pearls embolic member 8502 which is configured to be inserted into an aneurysm sac 8501; wherein the string-of-pearls embolic member further comprises a longitudinal series of embolic pieces which are connected to each other by one or more longitudinal wires, strands, cords, filaments, or coils; and wherein distal embolic pieces are closer together than proximal embolic pieces.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal wire, strand, cord, filament, or coil. In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along longitudinal wires, strands, cords, filaments, or coils. Relevant variations discussed elsewhere in this disclosure or in priority-linked disclosures can also be applied to this example.

FIG. 86 shows an example of a string-of-pearls embolic member which can be inserted into an aneurysm sac and/or a neck bridge in order to occlude (e.g. reduce blood flow within) the aneurysm sac. FIG. 86 shows a string-of-pearls embolic member 8602 which is configured to be inserted into an aneurysm sac 8601; wherein the string-of-pearls embolic member further comprises a longitudinal series of embolic pieces which are connected to each other by one or more longitudinal wires, strands, cords, filaments, or coils; and wherein distal embolic pieces are less-compressible and/or higher-durometer than proximal embolic pieces.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal wire, strand, cord, filament, or coil. In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along longitudinal wires, strands, cords, filaments, or coils. Relevant variations discussed elsewhere in this disclosure or in priority-linked disclosures can also be applied to this example.

FIG. 87 shows an example of a string-of-pearls embolic member which can be inserted into an aneurysm sac and/or a neck bridge in order to occlude (e.g. reduce blood flow within) the aneurysm sac. FIG. 87 shows a string-of-pearls embolic member 8702 which is configured to be inserted into an aneurysm sac 8701; wherein the string-of-pearls embolic member further comprises a longitudinal series of embolic pieces which are connected to each other by one or more longitudinal wires, strands, cords, filaments, or coils; and wherein proximal embolic pieces have more-irregular shapes than distal embolic pieces.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal wire, strand, cord, filament, or coil. In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along longitudinal wires, strands, cords, filaments, or coils. Relevant variations discussed elsewhere in this disclosure or in priority-linked disclosures can also be applied to this example.

FIG. 88 shows an example of a string-of-pearls embolic member which can be inserted into an aneurysm sac and/or a neck bridge in order to occlude (e.g. reduce blood flow within) the aneurysm sac. FIG. 88 shows a string-of-pearls embolic member 8802 which is configured to be inserted into an aneurysm sac 8801; wherein the string-of-pearls embolic member further comprises a longitudinal series of embolic pieces which are connected to each other by one or more undulating (e.g. sinusoidal) wires, strands, cords, filaments, or coils.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single undulating (e.g. sinusoidal) wire, strand, cord, filament, or coil. In an example, pairs of embolic pieces in the longitudinal series can be connected by different undulating wires, strands, cords, filaments, or coils. In an example, the centers of embolic pieces in the longitudinal series can be connected by one or more undulating wires, strands, cords, filaments, or coils. In an example, one or more undulating wires, strands, cords, filaments, or coils can be connected to the cross-sectional centers of embolic pieces. In an example, longitudinal strands, cords, filaments, or coils can be elastic and/or stretchable. In an example, a string-of-pearls embolic member can be structured so that embolic pieces can slide (a limited distance) along undulating wires, strands, cords, filaments, or coils. Relevant variations discussed elsewhere in this disclosure or in priority-linked disclosures can also be applied to this example.

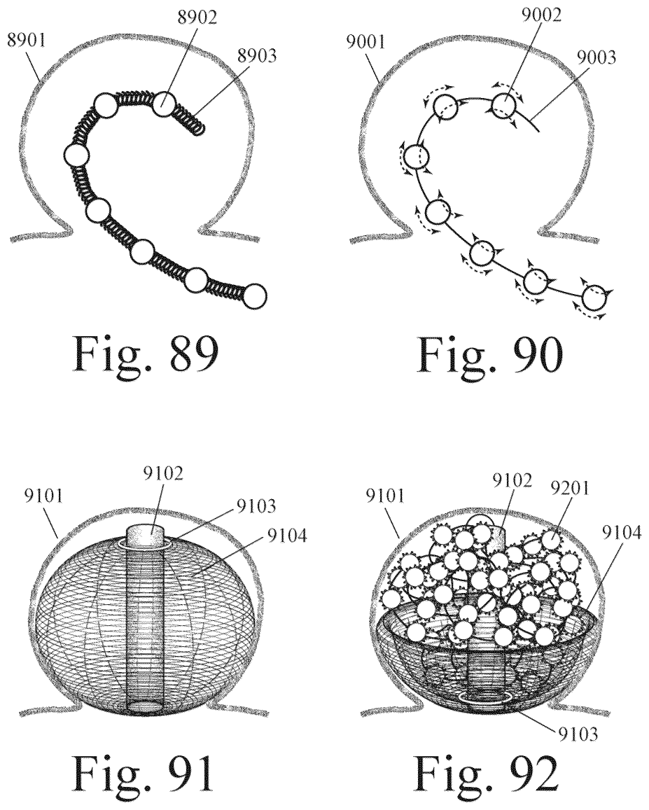

FIG. 89 shows an example of a string-of-pearls embolic member which can be inserted into an aneurysm sac and/or a neck bridge in order to occlude (e.g. reduce blood flow within) the aneurysm sac. FIG. 89 shows a string-of-pearls embolic member 8902 which is configured to be inserted into an aneurysm sac 8901; wherein the string-of-pearls embolic member further comprises a longitudinal series of embolic pieces which are connected to each other by one or more longitudinal coils.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces in a longitudinal series can all be connected along a single longitudinal coil. In an example, pairs of embolic pieces in the longitudinal series can be connected by different longitudinal coils. In an example, the centers of embolic pieces in the longitudinal series can be connected by one or more longitudinal coils. In an example, one or more longitudinal coils can be connected to the cross-sectional centers of embolic pieces. In an example, distal coils (or coil portions) can be less-flexible or less-compliant than proximal coils (or coil portions). Relevant variations discussed elsewhere in this disclosure or in priority-linked disclosures can also be applied to this example.

FIG. 90 shows an example of a string-of-pearls embolic member which can be inserted into an aneurysm sac and/or a neck bridge in order to occlude (e.g. reduce blood flow within) the aneurysm sac. FIG. 90 shows a string-of-pearls embolic member 9002 which is configured to be inserted into an aneurysm sac 9001; wherein the string-of-pearls embolic member further comprises a longitudinal series of embolic pieces which are connected to each other by one or more longitudinal wires, strands, cords, filaments, or coils; and wherein proximal embolic pieces can slide along the one or more longitudinal wires, strands, cords, filaments, or coils.

In an example, embolic pieces can be selected from the group consisting of: foam pieces, gelatin pieces, hydrogel pieces, microbeads, microsponges, miniature mesh balls, polymer polyhedrons, and polyvinyl alcohol pieces. In an example, embolic pieces can slide back and forth a limited distance along one or more longitudinal wires, strands, cords, filaments, or coils. In an example, this limited distance can be less than five times the diameter of an embolic piece. In an example, this limited distance can be between 1 and 4 times the average diameter of embolic pieces.

In an example, embolic pieces in a longitudinal series can all be connected along a single longitudinal wire, strand, cord, filament, or coil. In an example, pairs of embolic pieces in the longitudinal series can be connected by different longitudinal wires, strands, cords, filaments, or coils. In an example, the centers of embolic pieces in the longitudinal series can be connected by one or more longitudinal wires, strands, cords, filaments, or coils. In an example, one or more longitudinal wires, strands, cords, filaments, or coils can be connected to the cross-sectional centers of embolic pieces. In an example, longitudinal strands, cords, filaments, or coils can be elastic and/or stretchable. Relevant variations discussed elsewhere in this disclosure or in priority-linked disclosures can also be applied to this example.

FIGS. 91 and 92 show views at two different times of an example of an intrasaccular aneurysm occlusion device comprising a stent, a central column (e.g. funnel, tube, and/or lumen), and embolic pieces (or flowable material).

FIG. 91 shows this device at a first time in a first configuration wherein the stent is convex (e.g. globular) and the central column (e.g. funnel, tube, and/or lumen) is primarily within the stent. In an example, the distal end of the column can protrude out from the distal surface of the stent in the first configuration. In an example, the column can span the central proximal-to-distal axis of the stent in the first configuration.

FIG. 92 shows this device at a second time in a second configuration wherein the stent has been proximally compressed and inverted into a concave (e.g. bowl) shape and the central column is primarily outside the stent. In this example, the device is changed from the first configuration to the second configuration by the insertion of embolic pieces (or flowable material) through the column into the aneurysm sac. Accumulation of embolic pieces (or flowable material) in the aneurysm sac pushes the distal surface of the stent proximally, thereby proximally compressing and inverting the stent from a convex (e.g. globular) shape to a concave (e.g. bowl) shape.

With respect to specific components, FIGS. 91 and 92 show an intrasaccular aneurysm occlusion device comprising: a stent 9104 which is configured to be inserted into an aneurysm sac 9101; a central column (e.g. funnel, tube, and/or lumen) 9102; an annular member and/or opening in the stent 9103 through which the central column can slide; and embolic pieces (or flowable material) 9201 which are inserted through the central column into the aneurysm sac; wherein the device has a first configuration in which the stent has a convex (e.g. globular) shape and the central column is primarily within the stent, wherein the device has a second configuration in which the stent has a concave (e.g. bowl) shape and the central column is primarily outside the stent, and wherein the device is changed from the first configuration to the second configuration by insertion of the embolic pieces (or flowable material) through the central column into the aneurysm sac. In this example, the embolic pieces are string-of-pearl embolic strands. Relevant variations discussed elsewhere in this disclosure or in priority-linked disclosures can also be applied to this example.

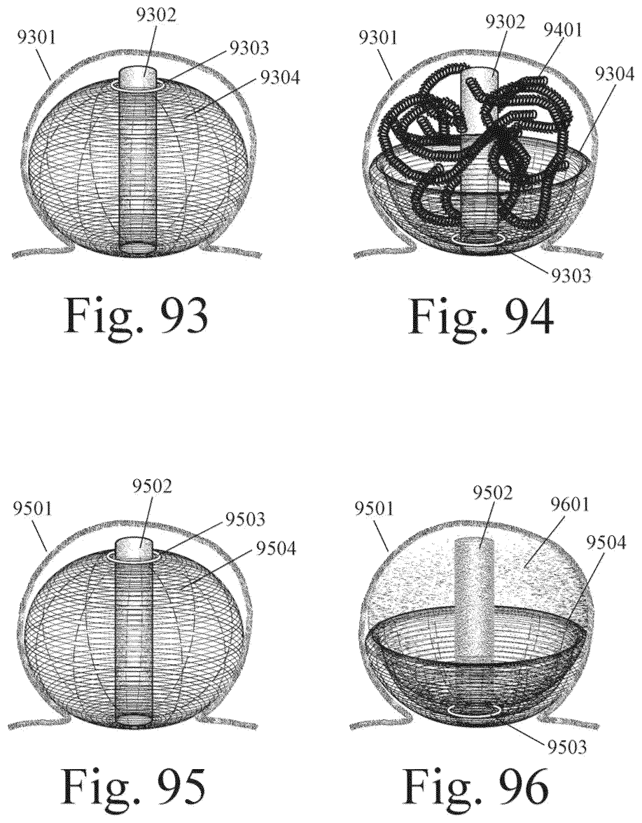

FIGS. 93 and 94 show views at two different times of an example of an intrasaccular aneurysm occlusion device comprising a stent, a central column (e.g. funnel, tube, and/or lumen), and embolic coils.

FIG. 93 shows this device at a first time in a first configuration wherein the stent is convex (e.g. globular) and the central column (e.g. funnel, tube, and/or lumen) is primarily within the stent. In an example, the distal end of the column can protrude out from the distal surface of the stent in the first configuration. In an example, the column can span the central proximal-to-distal axis of the stent in the first configuration.

FIG. 94 shows this device at a second time in a second configuration wherein the stent has been proximally compressed and inverted into a concave (e.g. bowl) shape and the central column is primarily outside the stent. In this example, the device is changed from the first configuration to the second configuration by the insertion of embolic coils through the column into the aneurysm sac. Accumulation of embolic coils in the aneurysm sac pushes the distal surface of the stent proximally, thereby proximally compressing and inverting the stent from a convex (e.g. globular) shape to a concave (e.g. bowl) shape.

With respect to specific components, FIGS. 93 and 94 show an intrasaccular aneurysm occlusion device comprising: a stent 9304 which is configured to be inserted into an aneurysm sac 9301; a central column (e.g. funnel, tube, and/or lumen) 9302; an annular member and/or opening in the stent 9303 through which the central column can slide; and embolic coils 9401 which are inserted through the central column into the aneurysm sac; wherein the device has a first configuration in which the stent has a convex (e.g. globular) shape and the central column is primarily within the stent, wherein the device has a second configuration in which the stent has a concave (e.g. bowl) shape and the central column is primarily outside the stent, and wherein the device is changed from the first configuration to the second configuration by insertion of the embolic coils through the central column into the aneurysm sac. Relevant variations discussed elsewhere in this disclosure or in priority-linked disclosures can also be applied to this example.

FIGS. 95 and 96 show views at two different times of an example of an intrasaccular aneurysm occlusion device comprising a stent, a central column (e.g. funnel, tube, and/or lumen), and embolic coils.

FIG. 95 shows this device at a first time in a first configuration wherein the stent is convex (e.g. globular) and the central column (e.g. funnel, tube, and/or lumen) is primarily within the stent. In an example, the distal end of the column can protrude out from the distal surface of the stent in the first configuration. In an example, the column can span the central proximal-to-distal axis of the stent in the first configuration.

FIG. 96 shows this device at a second time in a second configuration wherein the stent has been proximally compressed and inverted into a concave (e.g. bowl) shape and the central column is primarily outside the stent. In this example, the device is changed from the first configuration to the second configuration by the insertion of embolic flowable material (e.g. congealing liquid or gel) through the column into the aneurysm sac. Accumulation of embolic flowable material in the aneurysm sac pushes the distal surface of the stent proximally, thereby proximally compressing and inverting the stent from a convex (e.g. globular) shape to a concave (e.g. bowl) shape.

With respect to specific components, FIGS. 95 and 96 show an intrasaccular aneurysm occlusion device comprising: a stent 9504 which is configured to be inserted into an aneurysm sac 9501; a central column (e.g. funnel, tube, and/or lumen) 9502; an annular member and/or opening in the stent 9503 through which the central column can slide; and embolic flowable material 9601 which is inserted through the central column into the aneurysm sac; wherein the device has a first configuration in which the stent has a convex (e.g. globular) shape and the central column is primarily within the stent, wherein the device has a second configuration in which the stent has a concave (e.g. bowl) shape and the central column is primarily outside the stent, and wherein the device is changed from the first configuration to the second configuration by insertion of the embolic flowable material through the central column into the aneurysm sac. Relevant variations discussed elsewhere in this disclosure or in priority-linked disclosures can also be applied to this example.

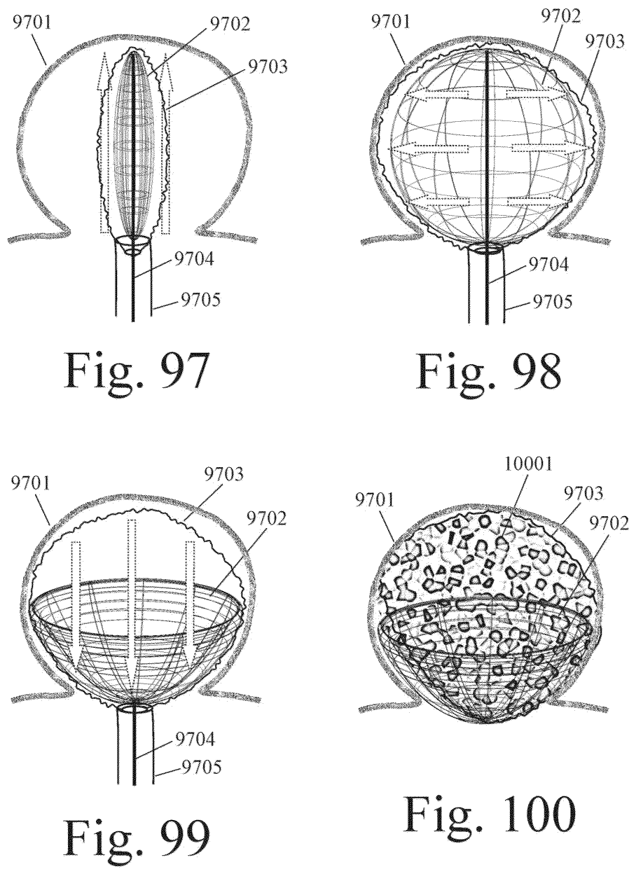

FIGS. 97 through 100 show four sequential views of deployment of an intrasaccular aneurysm occlusion device wherein a one-layer globular (e.g. spherical or ball-shaped) stent is collapsed and inverted into a two-layer concave (e.g. hemispherical or bowl-shaped) stent. The two-layer concave stent forms a double-layer barrier across an aneurysm neck (e.g. serving as a neck bridge).

This intrasaccular aneurysm occlusion device comprises: a proximal globular (e.g. spherical or ball-shaped) stent 9702 which is configured to be inserted and then collapsed and inverted into a concave (e.g. hemispherical or bowl-shaped) stent across an aneurysm neck within an aneurysm sac 9701; a distal flexible expanding member (e.g. mesh or net) 9703 within the aneurysm sac; and embolic pieces (or flowable material) 10001 which are inserted through the stent into the flexible expanding member (e.g. mesh or net). This example also includes a wire 9704 which is connected to the stent and a catheter 9705 through which the device is delivered to the aneurysm sac.

FIG. 97 shows this device exiting a catheter into an aneurysm sac. FIG. 98 shows the cross-sectional (e.g. radial) width of the globular (e.g. spherical or ball-shaped) stent expanding within the aneurysm sac. In an example, it can self-expand. FIG. 99 shows the stent being collapsed and inverted into a two-layer concave (e.g. hemispherical or bowl) shape. FIG. 100 shows a flexible expanding member (e.g. mesh or net) having been filled with embolic pieces (or flowable material) and now conforming to the walls of the aneurysm sac. Also, in FIG. 100 the catheter has been removed.

This aneurysm occlusion device can also be described as comprising: a proximal stent which is configured to be inserted into an aneurysm sac, wherein the proximal stent has a convex (e.g. globular, spherical, and/ball-shaped) first configuration and a concave (e.g. hemispherical or bowl-shaped) second configuration, and wherein the proximal stent is expanded into its convex first configuration within an aneurysm sac and then compressed and inverted into its concave second configuration across the aneurysm neck; a distal flexible mesh or net within the aneurysm sac; and embolic pieces (or flowable material) which are inserted into and retained within the distal flexible mesh or net. In an example, the stent can be compressed and inverted by pulling a wire. In another example, the stent can be compressed and inverted by pressure from the insertion of the embolic pieces (or flowable material) into the distal flexible mesh or net.

This aneurysm occlusion device can also be described as comprising: a proximal stent which is configured to be inserted into an aneurysm sac, wherein the proximal stent has a convex (e.g. globular, spherical, and/ball-shaped) first configuration and a concave (e.g. hemispherical or bowl-shaped) second configuration, and wherein the proximal stent is expanded into its convex first configuration within an aneurysm sac and then compressed and inverted into its concave second configuration across the aneurysm neck; a distal flexible mesh or net within the aneurysm sac; and embolic pieces (or flowable material) which are inserted into and retained within the distal flexible mesh or net, wherein insertion of the embolic pieces (or flowable material) into the distal flexible mesh or net expands and flexible mesh or net, and wherein insertion of the embolic pieces (or flowable material) into the distal flexible mesh or net also compresses and inverts the proximal stent from the convex first configuration to the concave second configuration.

In an example, the pre-compression convex shape of the stent can be selected from the group of shape consisting of: spherical, globular, ellipsoidal, pumpkin shape, apple shape, egg shape, and pear shape. In an example, a stent can be radially-expanded to be wider than an aneurysm neck in order to prevent the stent from coming out of the aneurysm sac after expansion. In an example, a stent can be a self-expanding metal or polymer structure. In an example, a stent can be a self-expanding wire mesh, net, or lattice. In an example, a stent can have be radially-compressed as it travels through a catheter to an aneurysm sac. In an example, a stent can have a proximal opening through which embolic pieces (or flowable material) are inserted. In an example, this opening can be closed after embolic pieces (or flowable material) have been inserted. In an example, it can further comprise a (one-way) valve through which embolic pieces (or flowable material) are inserted.

In an example, a flexible expanding member (e.g. mesh or net) can be selected from the group consisting of: mesh, net, liner, bag, and lattice. In an example, it can be elastic and/or stretchable. In an example, it can be folded and/or pleated in its first configuration. In an example, it can conform to the walls of an even an irregular-shaped aneurysm sac in order to prevent blood from flowing around device into the sac. In an example, a flexible expanding member (e.g. mesh or net) can have a proximal opening through which embolic pieces (or flowable material) are inserted. In an example, this opening can be closed after it has been expanded. In an example, it can further comprise a (one-way) valve through which embolic pieces (or flowable material) are inserted.

In an example, embolic pieces can be selected from the group consisting of: compressible balls or microspheres; rigid balls or microspheres; sponges, hydrogels, or pieces of foam; 3D polygons; string-of-pearls embolic strands; sinusoidal or otherwise undulating ribbons; and embolic coils. In an example, a flexible expanding member (e.g. mesh or net) can be expanded by filling it with embolic pieces. In an example, a flexible expanding member (e.g. mesh or net) can have pores (e.g. holes) in its perimeter, but these pores are smaller than embolic pieces so that that embolic pieces are retained within the flexible expanding member. In an example, a flexible expanding member (e.g. mesh or net) can be expanded by filling it with a liquid or gel. In an example, an embolic flowable material can be congealing liquid or gel.

In an variation on this example, an intrasaccular aneurysm occlusion device can comprise: a proximal stent which is configured to be inserted into an aneurysm sac, wherein the proximal stent has a convex (e.g. spherical, globular, and/or ball-shaped) first configuration and a concave (e.g. hemispherical and/or bowl-shaped) second configuration, and wherein the proximal stent is expanded into its convex first configuration within an aneurysm sac and then compressed and inverted into its concave second configuration across the aneurysm neck; and embolic pieces (or flowable material) which are inserted through the stent into the aneurysm sac, wherein insertion of the embolic pieces (or flowable material) into the aneurysm sac compresses and inverts the proximal stent from the first configuration to the second configuration.

In an example, a method for deploying an intrasaccular aneurysm occlusion device can comprise: inserting a proximal stent and a distal flexible mesh or net into an aneurysm sac, wherein the proximal stent has a convex (e.g. spherical, globular, and/or ball-shaped) first configuration, and wherein the proximal stent self-expands to a diameter which is greater than the diameter of the neck of the aneurysm sac after it has been inserted into the aneurysm sac; and inserting embolic pieces (or flowable material) through the proximal stent into the distal flexible mesh or net, wherein insertion of the embolic pieces (or flowable material) into the distal flexible mesh or net compresses and inverts the proximal stent into a concave (e.g. hemispherical and/or bowl-shaped) second configuration.

In an example, a method for deploying an intrasaccular aneurysm occlusion device can comprise: inserting a proximal stent and a distal flexible mesh or net into an aneurysm sac, wherein the proximal stent has a convex (e.g. spherical, globular, and/or ball-shaped) first configuration; expanding the proximal stent to a diameter which is greater than the diameter of the neck of the aneurysm sac after it has been inserted into the aneurysm sac; and inserting embolic pieces (or flowable material) through the proximal stent into the distal flexible mesh or net, wherein insertion of the embolic pieces (or flowable material) into the distal flexible mesh or net compresses and inverts the proximal stent into a concave (e.g. hemispherical and/or bowl-shaped) second configuration.

In an example, a method for deploying an intrasaccular aneurysm occlusion device can comprise: inserting a proximal stent and a distal flexible mesh or net into an aneurysm sac, wherein the proximal stent has a convex (e.g. spherical, globular, and/or ball-shaped) first configuration, and wherein the proximal stent self-expands to a diameter which is greater than the diameter of the neck of the aneurysm sac after it has been inserted into the aneurysm sac; compressing and inverting the proximal stent into a concave (e.g. hemispherical and/or bowl-shaped) configuration; and inserting embolic pieces (or flowable material) through the proximal stent into the distal flexible mesh or net, wherein insertion of the embolic pieces (or flowable material) expands the distal flexible mesh or net.

In an example, a method for deploying an intrasaccular aneurysm occlusion device can comprise: inserting a stent into an aneurysm sac, wherein the proximal stent has a convex (spherical, globular, and/or ball-shaped) first configuration; expanding the proximal stent to a diameter which is greater than the diameter of the neck of the aneurysm sac after it has been inserted into the aneurysm sac; and then inserting embolic pieces (or flowable material) through a (central) opening in the proximal stent into the aneurysm sac.

In an example, a method for deploying an intrasaccular aneurysm occlusion device can comprise: inserting a stent into an aneurysm sac, wherein the proximal stent has a convex (e.g. spherical, globular, and/or ball-shaped) first configuration, and wherein the proximal stent self-expands to a diameter which is greater than the diameter of the neck of the aneurysm sac; and inserting embolic pieces (or flowable material) through an opening in the proximal stent into the aneurysm sac. Relevant design and method variations discussed elsewhere in this disclosure or in priority-linked disclosures can also be applied to the example shown here.

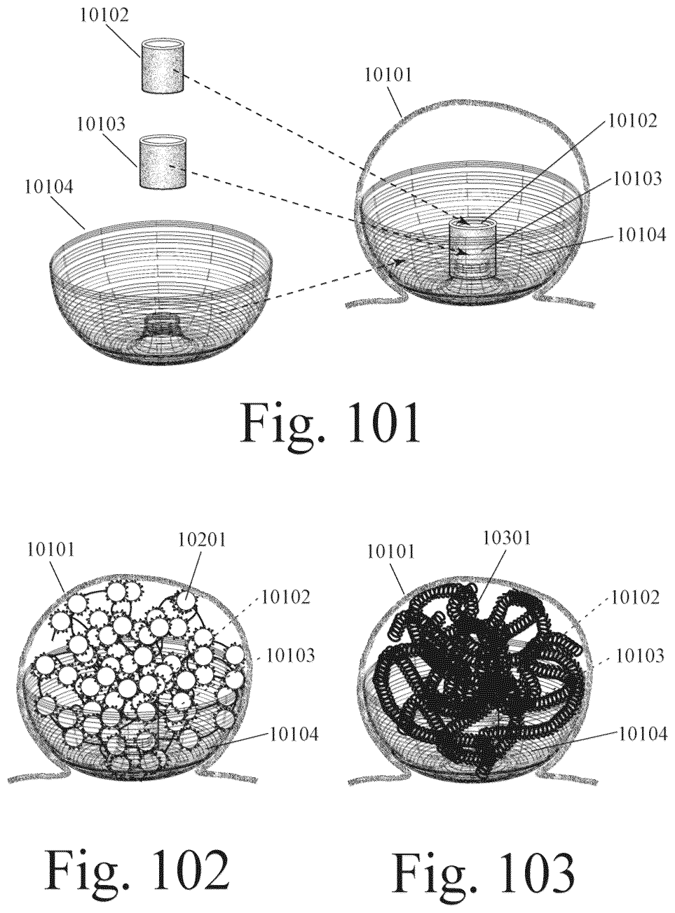

The left and right portions of FIG. 101 show two views of an example of an intrasaccular aneurysm occlusion device comprising: a partial-torus neck bridge 10104 which is configured to be inserted and expanded within an aneurysm sac to cover the neck of an aneurysm 10101, wherein the neck bridge further comprises a central funnel (e.g. funnel, indentation, or column) which extends distally from the (otherwise concave) proximal surface of the neck bridge; an inner annular member (e.g. ring, band, washer, column, or tube) 10102; and an outer annular member (e.g. ring, band, washer, column, or tube) 10103; wherein the inner annular member and the inner annular member are nested (e.g. concentric and/or coaxial), wherein portions (e.g. ends) of the neck bridge are inserted (e.g. held, bound, and/or pinched) between the inner annular member and the outer annular member, and wherein embolic pieces (or flowable material) can be inserted through the central opening of the inner annular member into the aneurysm sac. The left side of FIG. 101 shows the components (e.g. neck bridge and annular members) of this device displayed separately. The right side of FIG. 101 shows these components (e.g. neck bridge and annular members) combined, wherein the device has been inserted into an aneurysm sac.

FIG. 102 shows the device shown in FIG. 101 with the addition of embolic pieces (e.g. string-pearls embolic strands in this example) 10201 which have been inserted into the aneurysm sac through the central opening of the inner annular member. FIG. 103 shows the device shown in FIG. 101 with the addition of embolic coils 10301 which have been inserted into the aneurysm sac through the central opening of the inner annular member.

A partial-torus neck bridge has a shape which is a portion, percentage, and/or section of a torus shape. A torus is formed by revolving a convex shape around an axis which is coplanar with the convex shape and outside the convex shape. A partial-torus can be formed by revolving a section of this convex shape around this axis. In an example, a partial-torus neck bridge can have a shape which is between 20% and 50% of a torus. In an example, a partial-torus neck bridge can have a shape which can be modeled by revolving between 20% and 50% of a circle or ellipse around an axis which is coplanar with the circle or ellipse and outside the circle or ellipse. In an example, a lower-half of a torus can be formed by revolving the lower half of a circle or ellipse around an axis which is coplanar with the circle or ellipse and outside the circle or ellipse. In an example, a lower half of a torus can looks like the lower half of a sliced bagel. In an example, a partial-torus neck bridge can have a shape which can be modeled by between 50% and 100% of the lower half of a torus.

FIG. 101 shows an example of a partial-torus neck bridge with a shape similar to the lower half of a torus, except that the central funnel of the neck bridge is less distal (e.g. “shorter” in the figure) than the circumferential rim of the neck bridge. In an example, a partial-torus neck bridge with a shape similar to the lower half of a torus, except that the central funnel of the neck bridge is more distal (e.g. “higher” in the figure) than the circumferential rim of the neck bridge. In an example, the shape of a partial-torus neck bridge can be modeled by revolving an arc which is shaped like the letter “J” around an axis which is coplanar with the letter and a distance away from the letter. In an example, the shape of a partial-torus neck bridge can be modeled by revolving an arc which is shaped like a tilted letter “C” around an axis which is coplanar with the tilted letter and a distance away from the tilted letter. In an example, the shape of a partial-torus neck bridge can be modeled as a section (e.g. between 20% and 50%) of a torus which is formed by revolving an ellipse or oval around an axis which is: coplanar with the ellipse or oval; and outside the ellipse or oval. In an example, a partial-torus neck bridge can comprise convex-concave shape with an overall bowl shape and a local central funnel (e.g. funnel or indentation) extending distally from the center of the proximal surface of the bowl.

In an example, the diameter of the local central funnel can be between 5% and 35% of the diameter of the overall neck bridge. In an example, the diameter of the local central funnel can be between 20% and 50% of the diameter of the overall neck bridge. In an example, the height (e.g. distal distance) of the local central funnel can be between 25% and 75% of the height (e.g. distal distance) of the circumferential rim of the overall concave shape. In an example, a partial-torus neck bridge can have a single layer. In an example, a partial-torus neck bridge can be made from a tubular mesh. In an example, a partial-torus neck bridge can have two layers. In an example, a two-layer partial-torus neck bridge can be made by inverting a tubular mesh. In an example, an end of a partial-torus neck bridge can be inserted, held, bound, and/or pinched between an inner annular member and an outer annular member. In an example, two ends of a two-layer partial-torus neck bridge can be inserted, held, bound, and/or pinched between an inner annular member and an outer annular member. In an example, a partial-torus neck bridge can comprise wires and/or metal tubes. In an example, a partial-torus neck bridge can comprise a polymer mesh, matrix, or frame. In an example, a partial-torus neck bridge can be made by braiding or weaving. In an example, a partial-torus neck bridge can be made by 3D printing. In an example, a partial-torus neck bridge can be made by laser cutting.

In an example, the neck bridge can further comprise a valve or other closure mechanism in the central opening of the inner annular member, wherein this valve or other closure mechanism can be remotely opened or closed by the operator of the device (e.g. by the application of electrical energy, moving a wire, and/or actuating a microscale actuator such as MEMS component). In an example, a device can further comprise a closure mechanism (e.g. valve, plug, clip, or loop) which can be used (e.g. controlled by a device operator) to close the opening after embolic pieces (or flowable material) have been inserted through it into the aneurysm sac. In an example, a closure mechanism can be activated by the application of electrical energy, by pulling or pushing a wire, by rotating a wire, by pushing a plug, or by pumping congealing material. In an example, embolic pieces (or flowable material) which are inserted through an opening into an aneurysm sac can be selected from the group consisting of: micro-sponges; compressible micro-balls; embolic beads; pieces of foam; pieces of hydrogel; string-of-pearls embolic strands (e.g. embolic pieces connected by flexible longitudinal filaments, wires, coils, or threads); embolic coils; embolic ribbons; and liquid embolic material (e.g. flowable liquid or gel that congeals within the sac). Relevant variations discussed elsewhere in this disclosure or in priority-linked disclosures can also be applied to this example.

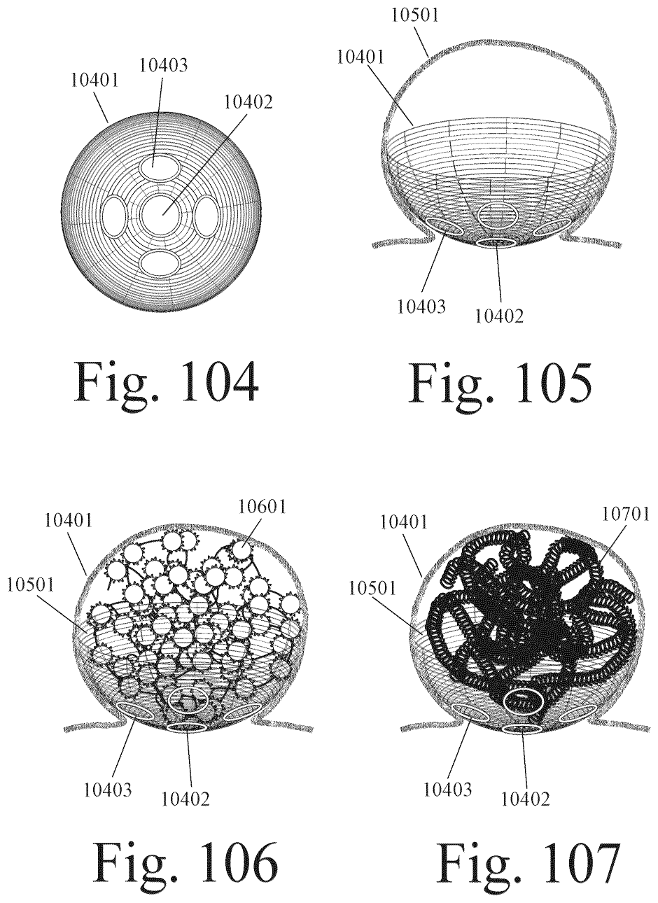

FIGS. 104 and 105 show two views of an example of an intrasaccular aneurysm occlusion device comprising: a convex (e.g. hemispherical or bowl-shaped) neck bridge 10401 which is configured to be inserted and expanded within an aneurysm sac 10501 to cover the neck of the aneurysm; a central opening 10402 in the neck bridge through which embolic pieces (or flowable material) can be inserted into the aneurysm sac; and a plurality of non-central openings (including 10403) in the neck bridge through which embolic pieces (or flowable material) can be inserted into the aneurysm sac.

FIG. 106 shows the device shown in FIGS. 104 and 105 with the addition of embolic pieces (e.g. string-pearls embolic strands in this example) 10601 which have been inserted into the aneurysm sac through one of the openings in the neck bridge. FIG. 107 shows the device shown in FIGS. 104 105 with the addition of embolic coils 10701 which have been inserted into the aneurysm sac through one of the openings in the neck bridge.

In an example, a neck bridge can have a hemispherical or bowl shape. In an example, a neck bridge can have one layer. In an example, a neck bridge can have two layers. In an example, a neck bridge can have two layers which are continuous at a circumferential fold and/or rim. In an example, a neck bridge can comprise metal wires or tubes. In an example, a neck bridge can comprise a polymer mesh or frame. In an example, a neck bridge can be made by braiding or weaving. In an example, a neck bridge can be made by laser cutting. In an example, a neck bridge can be made by 3D printing.

In an example, the central opening, the non-central openings, or both can be circular or elliptical. In an example, the central opening, the non-central openings, or both can have a polygonal perimeter. In an example, the plurality of non-central openings can be distributed in an arcuate manner (e.g. in a circle or ring) around the central opening. In an example, the plurality of non-central openings can equidistant from the central opening. In an example, non-central openings can be the same size as the central opening. In an example, non-central openings can be smaller than the central opening. In an example, non-central openings can be the same shape as the central opening.

In an example, the neck bridge can comprise a mesh, braid, or frame with a plurality of pores in addition to the central opening and the non-central openings, wherein a pore is less than half the size of an opening. In an example, the neck bridge can comprise a mesh, braid, or frame with a plurality of pores in addition to the central opening and the non-central openings, wherein a pore is smaller than an embolic piece but an opening is larger than an embolic piece, so that embolic pieces can be inserted the aneurysm sac through an opening, but embolic pieces do not escape out of the aneurysm sac through a pore.

In an example, a subset (e.g. one) of the central opening and the non-central openings can be selectively and remotely opened (or closed) by the device operator. In an example, the neck bridge can further comprise a plurality of valves and/or closure mechanisms which enable selective opening (and closing) of a subset of the central opening and the non-central openings. In an example, each of the central opening and the non-central openings can further comprise a valve or other closure mechanism which can be selectively and remotely opened (or closed). In an example, each of the central opening and the non-central openings can further comprise a valve or other closure mechanism which can be selectively and remotely opened (or closed) by a device operator by the application of electrical energy and/or activation of a microscale actuator. In an example, the device can further comprise a distal flexible net or mesh between the neck bridge and the dome of the aneurysm sac, wherein embolic pieces (or flowable material) are inserted through an opening into the net or mesh.

In an example, when the longitudinal axis of a neck bridge is aligned with the longitudinal axis of an aneurysms sac after insertion of the neck bridge into the aneurysm sac, then the operator of the device can insert embolic pieces (or flowable material) into the aneurysm sac through the central opening. In an example, when the longitudinal axis of a neck bridge is aligned with the longitudinal axis of an aneurysms sac after insertion of the neck bridge into the aneurysm sac, then the operator of the device can selectively open (e.g. open a valve or closure mechanism on) the central opening and insert embolic pieces (or flowable material) through the central opening into the aneurysm sac.

In an example, when the longitudinal axis of a neck bridge is not aligned with (e.g. at an acute angle with respect to) the longitudinal axis of an aneurysms sac after insertion of the neck bridge into the aneurysm sac, then the operator of the device can insert embolic pieces (or flowable material) into the aneurysm sac through a selected non-central opening. In an example, when the longitudinal axis of a neck bridge is aligned with the longitudinal axis of an aneurysms sac after insertion of the neck bridge into the aneurysm sac, then the operator of the device can selectively open (e.g. open a valve or closure mechanism on) a selected non-central opening and insert embolic pieces (or flowable material) through that non-central opening into the aneurysm sac. Relevant variations discussed elsewhere in this disclosure or in priority-linked disclosures can also be applied to this example.

I claim:

1. An intrasaccular aneurysm occlusion device comprising:
a proximal stent which is configured to be inserted into an aneurysm sac, wherein the proximal stent has a convex first configuration and a concave second configuration, and wherein the proximal stent is expanded into its convex first configuration within an aneurysm sac and then compressed and inverted into its concave second configuration across the aneurysm neck;
a distal flexible mesh or net within the aneurysm sac; and
embolic pieces which are or flowable material which is inserted into and retained within the distal flexible mesh or net.

2. The device in claim 1 wherein the convex first configuration is globular, spherical, and/or ball-shaped.

3. The device in claim 1 wherein the concave second configuration is hemispherical and/or bowl-shaped.

4. The device in claim 1 wherein the stent is compressed and inverted by pulling a wire.

5. The device in claim 1 wherein the stent is compressed and inverted by pressure from insertion of embolic pieces or flowable material into the distal flexible mesh or net.

6. The device in claim 1 wherein the stent has a proximal opening through which embolic pieces are or flowable material is inserted.

7. The device in claim 6 wherein the opening can be closed after embolic pieces are or flowable material is inserted.

8. An intrasaccular aneurysm occlusion device comprising:
a partial-torus neck bridge which is configured to be inserted and expanded within an aneurysm sac to cover the neck of an aneurysm, wherein the neck bridge further comprises a central funnel, indentation, and/or column which extends distally from the proximal surface of the neck bridge;
an inner annular member; and
an outer annular member, wherein the inner annular member and the inner annular member are nested, wherein portions of the neck bridge are inserted between the inner annular member and the outer annular member, and wherein embolic pieces or flowable material can be inserted through a central opening of the inner annular member into the aneurysm sac.

9. The device in claim 8 wherein the neck bridge is overall concave except for the central funnel, indentation, and/or column.

10. The device in claim 8 wherein the inner annular member is a ring, band, washer, column, or tube.

11. The device in claim 8 wherein the outer annular member is a ring, band, washer, column, or tube.

12. The device in claim 8 wherein the inner annular member and the outer annular member are concentric and/or coaxial.

13. The device in claim 8 wherein ends of the neck bridge are held, bound, and/or pinched between the inner annular member and the outer annular member.

14. The device in claim 8 wherein the neck bridge has two layers and ends of the two layers are held, bound, and/or pinched between the inner annular member and the outer annular member.

15. The device in claim 8 wherein the central funnel, indentation, or column of the neck bridge is less distal than a circumferential rim of the neck bridge.

16. An intrasaccular aneurysm occlusion device comprising:
a convex neck bridge which is configured to be inserted and expanded within an aneurysm sac to cover the neck of the aneurysm;
a central opening in the neck bridge through which embolic pieces or flowable material can be inserted into the aneurysm sac; and
a plurality of non-central openings in the neck bridge through which embolic pieces or flowable material can be inserted into the aneurysm sac.

17. The device in claim 16 wherein the neck bridge has a plurality of pores in addition to the central opening and the non-central openings, wherein a pore is less than half the size of an opening.

18. The device in claim 16 wherein the neck bridge has a plurality of pores in addition to the central opening and the non-central openings, wherein a pore is smaller than an embolic piece but an opening is larger than an embolic piece, so that embolic pieces can be inserted the aneurysm sac through an opening, but embolic pieces do not escape out of the aneurysm sac through a pore.

19. The device in claim 16 wherein a subset of the central opening and the non-central openings can be selectively and remotely opened by a device operator.

20. The device in claim 16 wherein the neck bridge further comprises a plurality of valves and/or closure mechanisms which enable selective opening of a subset of the central opening and the non-central openings.
